(19) United States
(12) Patent Application Publication
(10) Pub. No.: US 2026/0305610 A1
(43) Pub. Date: Oct. 8, 2026

(54) TRIAZOLE COMPOUND, AND PREPARATION METHOD THEREFOR, HERBICIDAL COMPOSITION AND USE THEREOF

(71) Applicant: QINGDAO KINGAGROOT CHEMICAL COMPOUND CO., LTD., Qingdao, Shandong (CN)

(72) Inventors: Lei LIAN, Qingdao, Shandong (CN); Rongbao HUA, Qingdao, Shandong (CN); Xuegang PENG, Qingdao, Shandong (CN); Qi CUI, Qingdao, Shandong (CN)

(21) Appl. No.: 19/163,310

(22) PCT Filed: Apr. 1, 2024
(86) PCT No.: PCT/CN2024/085264

§ 371 Date: Sep. 8, 2025

(30) Foreign Application Priority Data
Apr. 5, 2023 (CN) 202310357070.1
May 19, 2023 (CN) 202310575901.2
Aug. 28, 2023 (CN) 202311091881.8

(51) Int. Cl. A01N 43/653; A01P 13/00; C07D 401/04; C07D 401/14

(52) U.S. Cl. CPC A01N 43/653; A01P 13/00; C07D 401/04; C07D 401/14

(57) ABSTRACT

The present invention belongs to the technical field of pesticides, and particularly relates to a triazole compound, and a preparation method therefor, a herbicidal composition and the use thereof. The triazole compound is as shown in general formula I:

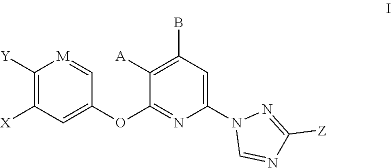

wherein M represents N or CH; X represents halogen, alkyl, haloalkyl, cyano, or haloalkoxy; Y represents hydrogen or halogen; Z represents hydrogen, halogen, cyano, or unsubstituted or halogen-substituted alkyl, alkenyl, alkynyl, cycloalkyl, alkoxy, alkenyloxy, alkynyloxy, alkoxyalkyl, alkylthio, alkylsulfoxy, or alkylsulfonyl; and A and B independently represent hydrogen, halogen, cyano, alkyl, cycloalkyl, alkoxy, alkylthio, alkylthioalkyl, or alkoxyalkyl, and A and B are not hydrogen at the same time. The compound has a good herbicidal activity on Gramineae weeds, broad-leaved weeds, Cyperaceae weeds, etc., even at a low application rate, and has high selectivity to crops.

### TECHNICAL FIELD

The present invention belongs to the technical field of pesticides and particularly relates to a triazole compound, as well as a preparation method, a herbicidal composition, and an application thereof.

### BACKGROUND ART

Weed control is one of the most important links in the course of achieving high-efficiency agriculture. Although various herbicides are available in the market, the herbicidal properties against harmful plants and the selectivity to crops of these known compounds are not completely satisfactory. Moreover, due to the continuous expansion of the market, issues such as resistance of weeds, service life and economy of drugs, as well as people's increasing attention to the environment, scientists are expected to continuously study and further develop new efficient, safe, and economical herbicide varieties with different modes of action.

### DESCRIPTION OF THE INVENTION

The present invention provides a triazole compound, as well as a preparation method, a herbicidal composition, and an application thereof. The compound has excellent herbicidal activity against gramineous weeds, broad-leaf weeds, cyperaceae weeds and the like even at a low application rate and has high selectivity to crops.

The technical scheme adopted in the present invention is as follows:

A triazole compound is as shown in the general formula I:

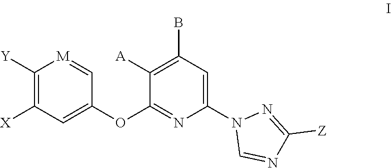

wherein, M represents N or CH;
X represents halogen, alkyl, haloalkyl, cyano, or haloalkoxy;
Y represents hydrogen or halogen;
Z represents hydrogen, halogen, cyano, or unsubstituted or halogen-substituted alkyl, alkenyl, alkynyl, cycloalkyl, alkoxy, alkenyloxy, alkynyloxy, alkoxyalkyl, alkylthio, alkylsulfoxide, or alkylsulfonyl;
A and B each independently represent hydrogen, halogen, cyano, alkyl, cycloalkyl, alkoxy, alkylthio, alkylthioalkyl, or alkoxyalkyl. Also, A and B are not simultaneously hydrogen.

In one specific embodiment, X represents halogen, C1-C8 alkyl, halo C1-C8 alkyl, cyano, or halo C1-C8 alkoxy;
Z represents hydrogen, halogen, cyano, or unsubstituted or halogen-substituted C1-C8 alkyl, C2-C8 alkenyl, C2-C8 alkynyl, C3-C8 cycloalkyl, C1-C8 alkoxy, C2-C8 alkenyloxy, C2-C8 alkynyloxy, C1-C8 alkoxy C1-C8 alkyl, C1-C8 alkylthio, C1-C8 alkylsulfoxide, or C1-C8 alkylsulfonyl;
A and B each independently represent hydrogen, halogen, cyano, C1-C8 alkyl, C3-C8 cycloalkyl, C1-C8 alkoxy, C1-C8 alkylthio, C1-C8 alkylthio C1-C8 alkyl, or C1-C8 alkoxy C1-C8 alkyl.

In another specific embodiment, X represents halogen, C1-C6 alkyl, halo C1-C6 alkyl, cyano, or halo C1-C6 alkoxy;
Z represents hydrogen, halogen, cyano, or unsubstituted or halogen-substituted C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C1-C6 alkoxy C1-C6 alkyl, C1-C6 alkylthio, C1-C6 alkylsulfoxide, or C1-C6 alkylsulfonyl;
A and B each independently represent hydrogen, halogen, cyano, C1-C6 alkyl, C3-C6 cycloalkyl, C1-C6 alkoxy, C1-C6 alkylthio, C1-C6 alkylthio C1-C6 alkyl, or C1-C6 alkoxy C1-C6 alkyl.

In the definition of the compound represented by the above-mentioned general formula and all of the following structural formulas, the technical terms used, whether used alone or used in compound words, represent the following substituent groups: an alkyl group which has more than two carbon atoms and may be linear or branched. For example, the alkyl in the compound word “alkoxyalkyl” may be —CH₂—, —CH₂CH₂—, —CH(CH₃)—, —C(CH₃)₂—, etc. An alkyl group is, for example, C1 alkyl: methyl; C2 alkyl: ethyl; C3 alkyl: propyl such as n-propyl or isopropyl; C4 alkyl: butyl such as n-butyl, isobutyl, tert-butyl, or 2-butyl; C5 alkyl: pentyl such as n-pentyl; C6 alkyl: hexyl such as n-hexyl, isohexyl, and 1,3-dimethylbutyl. Similarly, an alkenyl is, for example, vinyl, allyl, 1-methylprop-2-en-1-yl, 2-methylprop-2-en-1-yl, but-2-en-1-yl, but-3-en-1-yl, 1-methylbut-3-en-1-yl, and 1-methylbut-2-en-1-yl. An alkynyl is, for example, ethynyl, propargyl, but-2-yn-1-yl, but-3-yn-1-yl, and 1-methylbut-3-yn-1-yl. A multiple bond may be at any position of each unsaturated group. A cycloalkyl is a saturated carbocyclic ring system having, for example, three to six carbon atoms such as cyclopropyl, cyclobutyl, cyclopentyl, or cyclohexyl. Similarly, a cycloalkenyl is a monocyclic alkenyl having, for example, three to six carbon ring members such as cyclopropenyl, cyclobutenyl, cyclopentenyl, and cyclohexenyl, wherein a double bond can be at any position. A halogen is fluorine, chlorine, bromine, or iodine.

If one group is substituted by a group, which should be understood to mean that the group is substituted by one or more same or different groups selected from the mentioned groups. In addition, the same or different substitution characters contained in the same or different substituent groups are independently selected, which may be the same or different. This is also applicable to ring systems formed by different atoms and units. Meanwhile, the scope of the claims will exclude those compounds chemically unstable under standard conditions which are known to a person skilled in the art.

In addition, unless specifically defined, the “aryl” of the present invention includes, but is not limited to, phenyl, naphthyl,

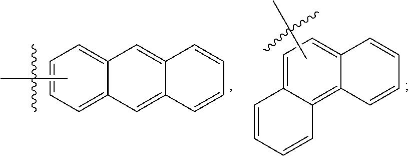

the term “substituted by at least one group” refers to being substituted by, for example, 1, 2, 3, 4, or 5 groups; a group without being specified a linking site may be linked at any position, including a position connected to C or N; if it is substituted, the substituent group may also substitute at any position as long as the valence bond theory is complied with.

According to the nature of substituent groups and the manner in which they are connected, the compound of the general formula I and derivatives thereof may exist as stereoisomers. For example, if one or more asymmetric carbon atoms are present, enantiomers and diastereoisomers may occur. Stereoisomers may be obtained by conventional separation methods, for example by chromatographic separation, from mixtures produced in preparation. Stereoisomers may also be selectively prepared by employing stereoselective reactions and using optically active starting materials and/or auxiliaries.

The present invention further relates to all stereoisomers which are included in the general formula I but not specifically defined, and mixtures thereof.

The preparation method of the triazole compound comprises the following steps:
subjecting the compound shown in the general formula II and the compound shown in the general formula III to substitution reaction to produce the compound shown in the general formula I, and the chemical reaction equation is as follows:

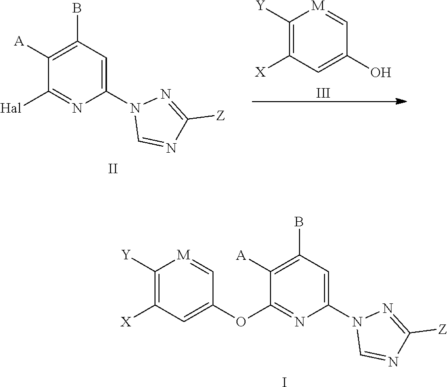

wherein, Hal represents halogen, preferably Cl; the definitions of substituents A, B, M, X, Y, and Z are as shown above.

Preferably, the reaction is carried out in the presence of a base and a solvent; more preferably, the base is selected from at least one of inorganic bases (e.g., K₂CO₃, Na₂CO₃, Cs₂CO₃, NaHCO₃, KF, CsF, KOAc, AcONa, K₃PO₄, t-BuONa, EtONa, NaOH, KOH, NaOMe, etc.) or organic bases (pyrazole or C1-C6 alkylamine, e.g., triethylamine, trimethylamine, N-ethyldiisopropylamine, DIEA, etc.); the solvent is selected from at least one of aromatic hydrocarbons (e.g., benzene, chlorobenzene, toluene, cresol, or o-, m-, and p-xylene), THF, DMF, DMA, methanol, ethanol, acetonitrile, dichloroethane, DMSO, dioxane, dichloromethane, toluene, or ethyl acetate.

A herbicidal composition comprises (i) a herbicidally effective amount of at least one of the triazole compounds; preferably, also comprises (ii) a formulation auxiliary.

In one specific embodiment, the herbicidal composition further comprises (iii) a herbicidally effective amount of one or more additional herbicides and/or safeners.

A method for controlling weeds comprises applying a herbicidally effective amount of at least one of the triazole compounds or the herbicidal composition to a plant or a weedy area.

At least one of the triazole compounds or the herbicidal composition has a use for controlling weeds; preferably, the triazole compound is used to control weeds among useful crops, and the useful crops are transgenic crops or crops treated by genome editing techniques.

The compound of Formula I of the present invention has outstanding herbicidal activity against a broad spectrum of monocotyledonous and dicotyledonous harmful plants of economic importance. The active substances of the present invention also act efficiently on perennial weeds which grow from root stocks, rhizomes, or other perennial organs and are difficult to control. In this context, it is generally immaterial whether the substances are applied before sowing, before emergence, or after emergence. Representative examples of monocotyledonous and dicotyledonous weed florae which can be controlled by the compound of the present invention may be specifically mentioned, without limiting to certain species. Examples of weed species on which the active substances act efficiently include the monocotyledons such as annual Avena, Lolium, Alopecurus, Phalaris, Echinochloa, Digitaria, Setaria, and Cyperus, as well as perennial Agropyron, Cynodon, Imperata and Sorghum, and perennial Cyperus.

In the case of the dicotyledonous weed species, the spectrum of action extends to species such as annual Galium, Viola, Veronica, Lamium, Stellaria, Amaranthus, Sinapis, Ipomoea, Sida, Matricaria, and Abutilon, as well as perennial weeds Convolvulus, Cirsium, Rumex, and Artemisia. The active substances of the present invention perform outstanding control of harmful plants such as Echinochloa, Sagittaria, Alisma, Eleocharis, Scirpus, and Cyperus under the specific condition of rice growing. If the compound of the present invention is applied to soil surface prior to germination, the weed seedlings are either prevented completely from emerging, or the weeds stop growing when reaching the cotyledon stage and eventually died completely after three to four weeks. In particular, the compound of the present invention exhibits excellent activity against Apera spica venti, Matsumurella chinense, Fallopia convolvulus, Stellaria media, Veronica hederifolia, Veronica persica, Viola tricolor, Amaranthus, Galium, and Kochia.

Although the compound of the present invention has excellent herbicidal activity against monocotyledonous and dicotyledonous weeds, crop plants of economic importance such as wheat, barley, rye, rice, corn, sugarbeet, cotton, and soya are not damaged at all, or only to a negligible extent. In particular, they have excellent compatibility with cereals such as wheat, barley, and corn, in particular wheat. For these reasons, the compound of the present invention is highly suitable for selectively controlling undesired plants in plantings for agricultural or ornamental use.

Owing to their herbicidal properties, these active substances may be employed for controlling harmful plants in plantings of genetically engineered plants that are known or to be introduced. Transgenic plants usually have advantageous traits, for example, resistance to certain pesticides, in particular to certain herbicides; resistance to plant diseases or pathogenic microorganisms of plant diseases such as certain insects or microorganisms including fungi, bacteria, or viruses. Other particular traits relate to the following conditions of the product, for example, quantity, quality, storage stability, composition, and special ingredients. Thus, it is known that the obtained transgenic plant product has an increased starch content, or modified starch quality, or a different fatty acid composition.

The compound of Formula I of the present invention or salts thereof are preferably used in transgenic crops and ornamental plants of economic importance, for example, cereals such as wheat, barley, rye, oats, millet, rice, manioc, and corn, or else in plantings of sugarbeet, cotton, soya, rapeseed, potato, tomato, pea, and other vegetable plants. The compound of Formula I is preferably used as a herbicide in plantings of useful plants which are resistant, or have been made resistant by genetic engineering, against the toxic effects of herbicides.

Conventional ways for breeding plants which have modified traits compared to known plants include, for example, conventional breeding methods and breeding of mutant strains. In other words, novel plants having improved traits may be generated with the aid of genetic engineering methods (see, e.g., EP-0221044 A, EP-0131624 A). For example, several methods have been described:
changing crop plants using genetic engineering to modify the starch synthesis in plants (e.g., WO 92/11376, WO 92/14827, WO 91/19806);
transgenic crop plants resistant to certain herbicides, including glufosinate herbicides (e.g., EP-0242236 A, EP-0242246 A), or glyphosate herbicides (WO 92/00377), or sulfonylurea herbicides (EP-0257993 A, U.S. Pat. No. 5,013,659 A);
transgenic crop plants, for example cotton, able to produce Bacillus thuringiensis toxins (Bt toxins) which impart resistance to certain pests that invade the plants (EP-0142924 A, EP-0193259 A);
transgenic crop plants having a modified fatty acid composition (WO 91/13972).

Numerous molecular biology techniques which allow the preparation of transgenic plants having modified traits are known (see, e.g., Sambrook et al., 1989, Molecular Cloning, A Laboratory Manual, 2nd edition, Cold Spring Harbor Laboratory Press, Cold Spring Harbor, N.Y.; or Winnacker, “Gene und Klone” [Genes and Clones], VCH Weinheim, 2nd edition, 1996; or Christou, “Trends in Plant Science”, 1 (1996) 423-431). In order to carry out genetic engineering manipulations, it is possible to introduce nucleic acid molecules into plasmids, allowing mutagenesis or changes in sequences to occur by recombination of DNA sequences. Using the above-mentioned standard processes, it is possible to, for example, substitute bases, remove partial sequences, or add natural or synthetic sequences. To link DNA fragments with each other, it is possible to attach adaptors or linkers to the fragments.

A plant cell having a gene product of reduced activity may be prepared by the following methods, for example, by expressing at least one appropriate antisense-RNA and one sense-RNA to achieve a cosuppression effect, or by expressing at least one appropriately constructed ribozyme which specifically cleaves transcripts of the above-mentioned gene product.

To this end, it is possible to employ a DNA molecule which comprises all the coding sequences of the gene product including any flanking sequence that may be present, and a DNA molecule which comprises only parts of the coding sequences that must be long enough to cause an antisense effect in the cells. It is also possible to use sequences which have a high degree of homology but are not entirely identical to the coding sequences of the gene product.

When nucleic acid molecules are expressed in a plant, the synthesized protein may be localized in any desired compartment of the plant cell. However, to achieve localization in a certain compartment, it is possible, for example, to link the coding region with the DNA sequences to ensure localization in the certain compartment. Such sequences are known to a person skilled in the art (see, e.g., Braun et al., EMBO J., 11 (1992), 3219-3227; Wolter et al., Proc. Natl. Acad. Sci. USA, 85 (1988), 846-850; Sonnewald et al., Plant J., 1 (1991), 95-106).

Transgenic plant cells can be recombined onto a whole plant using known techniques. A transgenic plant may be of any desired plant species, i.e., a monocotyledonous and dicotyledonous plant. In this manner, it is possible to obtain transgenic plants having modified traits by overexpression, suppression, or inhibition of homologous (=natural) genes or gene sequences, or by expression of heterologous (=foreign) genes or gene sequences.

When using the active substances of the present invention in transgenic crops, in addition to the inhibitory effect against harmful plants which can be observed in other crops, there are frequently special effects on the corresponding transgenic crops, for example, improving or broadening the spectrum of weeds which can be controlled, modifying the application amount during application, excellent combination of the drug resistance of preferred transgenic crops and herbicide performance, as well as effects on the growth and the yield of transgenic crop plants. The present invention therefore also provides a use of the compound as a herbicide for controlling harmful plants among transgenic crop plants.

In addition, the compound of the present invention is able to significantly regulate the growth of crop plants. These compounds are employed for the targeted control of plant constituents and for facilitating harvesting, for example by provoking desiccation and stunted growth, by engaging in plant metabolism in a regulating manner. Furthermore, they are also suitable for regulating and inhibiting undesirable plant growth without destroying the growth of crop plants. Inhibition of plant growth plays a key role in many monocotyledonous and dicotyledonous crops because it can reduce or completely prevent lodging hereby.

The compound of the present invention may be applied in customary formulations in the form of wettable powders, emulsifiable concentrates, sprayable solutions, dusts, or granules. The present invention therefore also provides herbicidal compositions comprising the compound of Formula I. The compound of Formula I may be formulated in numerous ways depending on the prevailing physical parameters in biology and/or chemistry. Examples of suitable formulation options are: wettable powders (WP), water-soluble powders (SP), water-soluble concentrates, emulsifiable concentrates (EC), emulsions such as oil-in-water and water-in-oil emulsions (EW), sprayable solutions, suspension concentrates (SC), oil dispersions (OD), oil- or water-based dispersions, oil-miscible solutions, dust powders (DP), capsule suspensions (CS), seed-dressing compositions, granules for broadcasting and soil application, spray granules, coating granules and adsorption granules, water-dispersible granules (WG), water-soluble granules (SG), ULV (Ultra-low volume) formulations, microcapsules, and waxes. These individual formulation types are known and are described in the following literature, for example, Winnacker-Küchler, “Chemische Technologie” [Chemical Technology], Volume 7, C. Hauser Verlag Munich, 4th edition, 1986; Wade van Valkenburg, “Pesticide Formulations”, Marcel Dekker, N.Y., 1973; K. Martens, “Spray Drying” Handbook, 3rd edition, 1979, G. Goodwin Ltd. London.

Necessary formulation auxiliaries such as inert materials, surfactants, solvents and other additives are likewise known, and are described in the following documents, for example, Watkins, “Handbook of Insecticide Dust Diluents and Carriers”, 2nd edition, Dorland Books, Caldwell N.J.; H. v. Olphen, “An Introduction to Clay Colloid Chemistry”, 2nd edition, J. Wiley & Sons, N.Y.; C. Marsden, “Solvents Guide”, 2nd edition, Interscience, N.Y. 1963; “Mccutcheon's Detergents and Emulsifiers Annual”, MC Publ. Corp., Ridgewood N.J.; Sisley and Wood, “Encyclopedia of Surface Active Agents”, Chem. Publ. Co. Inc., N.Y., 1964; Schönfeldt, “Grenzflüchenaktive Äthylenoxidaddkte” [Surface-active ethylene oxide adducts], Wiss. Verlagagesell., Stuttgart, 1976; Winnacker-Küchler, “Chemische Technologie” [Chemical Technology], Volume 7, C. Hauser Verlag Munich, 4th edition, 1986.

Wettable powders are uniformly dispersible in water which contain, in addition to the active substance, a diluent or inert substance, ionic and/or nonionic surfactant (wetting agent, dispersant), for example polyethoxylated alkyl phenol, polyethoxylated fatty alcohol, polyethoxylated fatty amine, fatty alcohol polyglycol ether sulfate, alkane sulfonate, alkylphenyl sulfonate, sodium lignosulfonate, sodium 2,2′-dinaphthylmethane-6,6′-disulfonats, sodium dibutylnaphthalenesulfonate, or sodium methyl oleoyl taurate. To prepare the wettable powders, the herbicidally active substance is finely ground, for example, in customary apparatus such as hammer mills, fan mills and air-jet mills, and mixed with formulation auxiliaries simultaneously or sequentially.

Emulsifiable concentrates are prepared by dissolving the active substance in an organic solvent, for example butanol, cyclohexanone, dimethylformamide, xylene or aromatic compounds with relatively high boiling point or hydrocarbons or mixtures of the solvents, with the addition of one or more ionic and/or nonionic surfactants (emulsifiers). Examples of emulsifiers which can be used are calcium alkylarylsulfonates such as calcium dodecylbenzene sulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters such as sorbitan fatty acid esters, or polyoxyethylene sorbitan esters such as polyoxyethylene sorbitan fatty acid esters.

Dusts are obtained by grinding the active substance with finely-divided solid substances, for example talc, natural clays such as kaolin, bentonite and pyrophyllite, or diatomaceous earth. Water- or oil-based suspensions may be prepared by the following methods, for example, by wet milling using commercially customary bead mills, with or without the addition of the surfactants mentioned above in the case of another formulation type.

Emulsions such as oil-in-water emulsions (EW) may be prepared by means of stirrers, colloid mills and/or static mixers using aqueous organic solvents and, if desired, the surfactants as mentioned above in the case of another formulation type may be added.

Granules may be prepared by the following methods that either spraying the active substance onto the adsorptive and granulating with inert materials, or concentrating the active substance onto the surface of carriers such as sand and kaolinite and granulating inert materials by means of adhesive binders such polyvinyl alcohol, sodium polyacrylate or mineral oils. Suitable active substances may also be granulated in the manner which is customary for the preparation of fertilizer granules, if desired, fertilizers may be mixed in. Water-dispersible granules are prepared by customary methods such as spray-drying, fluidized-bed granulation, disk granulation, mixing using high-speed mixers, and extrusion without solid inert materials.

For the preparation methods of granules using disk, fluidized-bed, extruder and spray, see the following processes in, for example, “Spray-Drying Handbook” 3rd edition, 1979, G. Goodwin Ltd., London; J. E. Browning, “Agglomeration”, Chemical and Engineering, 1967, pages 147 ff.; “Perry's Chemical Engineer's Handbook”, 5th edition, McGraw-Hill, New York 1973, pp. 8-57. For further details on the formulation of crop protection products, see, for example G. C. Klingman, “Weed Control as a Science”, John Wiley and Sons Inc., New York, 1961, pages 81-96 and J. D. Freyer, S. A. Evans, “Weed Control Handbook”, 5th edition, Blackwell Scientific Publications, Oxford, 1968, pages 101-103.

Agrochemical formulations generally contain from 0.1 to 99% by weight, in particular from 0.1 to 95%, of the active substance of Formula I. In wettable powders, the concentration of the active substance is, for example, from about 10 to 99% by weight, and the remainder to 100% by weight consists of customary formulation constituents. In emulsifiable concentrates, the concentration of the active substance may be from about 1 to 90%, preferably from 5 to 80%, by weight. Dust formulations contain from 1 to 30% by weight of the active substance, preferably from 5 to 20% by weight of the active substance in usual cases, while sprayable solutions contain from about 0.05 to 80%, preferably from 2 to 50%, by weight of the active substance. In the case of water-dispersible granules, the content of the active substance mainly depends on whether the active substance is liquid or solid and on auxiliaries, fillers, etc. that are used during granulation. In water-dispersible granules, the content of the active substance, for example, is between 1 and 95% by weight, preferably between 10 and 80% by weight.

In addition, the formulations of the active substance may include tackifiers, wetting agents, dispersants, emulsifiers, penetrants, preservatives, antifreeze agents, solvents, fillers, carriers, colorants, defoamers, evaporation suppressors as well as pH and viscosity modifiers which are usually customary in all cases.

Based on these formulations, it is also possible to produce mixtures with other pesticidally active substances, for example insecticides, acaricides, herbicides and fungicides, and also with safeners, fertilizers and/or plant growth regulators, in a manner of pre-mix or tank mix.

In mixed formulations or tank mix formulations, suitable active substances which can be mixed with the active substance of the present invention are the known substances described, for example in World Herbicide New Product Technology Handbook, China Agricultural Science and Farming Techniques Press, 2010.9 and in the literature cited herein. For example, the herbicidally active substances mentioned below may be mixed with the compound of Formula I (noted that the compounds are either named by the “common name” in accordance with the International Organization for Standardization (ISO) or by the chemical names, accompanied with a customary code number if appropriate): acetochlor, butachlor, alachlor, propisochlor, metolachlor, S-metolachlor, pretilachlor, propachlor, ethachlor, napropamide, R-left handed napropamide, propanil, mefenacet, diphenamid, diflufenican, ethaprochlor, beflubutamid, bromobutide, dimethenamid, dimethenamid-P, etobenzanid, flufenacet, thenylchlor, metazachlor, isoxaben, flamprop-M-methyl, flamprop-M-propyl, allidochlor, pethoxamid, chloranocryl, cypromid, mefluidide, monalide, delachlor, prynachlor, terbuchlor, xylachlor, dimethachlor, cisanilide, trimexachlor, clomeprop, propyzamide, pentanochlor, carbetamide, benzoylprop-ethyl, cyprazole, butenachlor, tebutam, benzipram, quinonamid, dichlofluanid, naproanilide, diethatyl-ethyl, naptalam, flufenacet, benzadox, chlorthiamid, chlorophthalimide, isocarbamide, picolinafen, atrazine, simazine, prometryn, cyanatryn, simetryn, ametryn, propazine, dipropetryn, SSH-108, terbutryn, terbuthylazine, triaziflam, cyprazine, proglinazine, trietazine, prometon, simetone, aziprotryne, desmetryn, dimethametryn, procyazine, mesoprazine, sebuthylazine, secbumeton, terbumeton, methoprotryne, cyanatryn, ipazine, chlorazine, atraton, pendimethalin, eglinazine, cyanuric acid, indaziflam, chlorsulfuron, metsulfuron-methyl, bensulfuron-methyl, chlorimuron-ethyl, tribenuron-methyl, thifensulfuron-methyl, pyrazosulfuron-ethyl, mesosulfuron, iodosulfuron-methyl sodium, foramsulfuron, cinosulfuron, triasulfuron, sulfometuron methyl, nicosulfuron, ethametsulfuron-methyl, amidosulfuron, ethoxysulfuron, cyclosulfamuron, rimsulfuron, azimsulfuron, flazasulfuron, monosulfuron, monosulfuron-ester, flucarbazone-sodium, flupyrsulfuron-methyl, halosulfuron-methyl, oxasulfuron, imazosulfuron, primisulfuron, propoxycarbazone, prosulfuron, sulfosulfuron, trifloxysulfuron, triflusulfuron-methyl, tritosulfuron, sodium metsulfuron methyl, flucetosulfuron, HNPC-C9908, orthosulfamuron, propyrisulfuron, metazosulfuron, acifluorfen, fomesafen, lactofen, fluoroglycofen, oxyfluorfen, chlornitrofen, aclonifen, ethoxyfen-ethyl, bifenox, nitrofluorfen, chlomethoxyfen, fluorodifen, fluoronitrofen, furyloxyfen, nitrofen, TOPE, DMNP, PPG1013, AKH-7088, halosafen, chlortoluron, isoproturon, linuron, diuron, dymron, fluometuron, benzthiazuron, methabenzthiazuron, cumyluron, ethidimuron, isouron, tebuthiuron, buturon, chlorbromuron, methyldymron, phenobenzuron, SK-85, metobromuron, metoxuron, afesin, monuron, siduron, fenuron, fluothiuron, neburon, chloroxuron, noruron, isonoruron, 3-cyclooctyl-1, thiazfluron, tebuthiuron, difenoxuron, parafluron, methylamine tribunil, karbutilate, trimeturon, dimefuron, monisouron, anisuron, methiuron, chloreturon, tetrafluron, phenmedipham, phenmedipham-ethyl, desmedipham, asulam, terbucarb, barban, propham, chlorpropham, rowmate, swep, chlorbufam, carboxazole, chlorprocarb, fenasulam, BCPC, CPPC, carbasulam, butylate, benthiocarb, vemolate, molinate, triallate, dimepiperate, esprocarb, pyributicarb, cycloate, avadex, EPTC, ethiolate, orbencarb, pebulate, prosulfocarb, tiocarbazil, CDEC, dimexano, isopolinate, methiobencarb, 2,4-D butyl ester, MCPA-Na, 2,4-D isooctyl ester, MCPA isooctyl ester, 2,4-D sodium salt, 2,4-D dimethyla mine salt, MCPA-thioethyl, MCPA, 2,4-D propionic acid, high 2,4-D propionic acid salt, 2,4-D butyric acid, MCPA propionic acid, MCPA propionic acid salt, MCPA butyric acid, 2,4,5-D, 2,4,5-D propionic acid, 2,4,5-D butyric acid, MCPA amine salt, dicamba, erbon, chlorfenac, saison, TBA, chloramben, methoxy-TBA, diclofop-methyl, fluazifop-butyl, fluazifop-P-butyl, haloxyfop-methyl, haloxyfop-P, quizalofop-ethyl, quizalofop-P-ethyl, fenoxaprop-ethy, fenoxaprop-P-ethyl, propaquizafop, cyhalofop-butyl, metamifop, clodinafop-propargyl, fenthiapropethyl, chloroazifop-propynyl, poppenate-methyl, trifopsime, isoxapyrifop, paraquat, diquat, oryzalin, ethalfluralin, isopropalin, nitralin, profluralin, prodinamine, benfluralin, fluchloraline, dinitramina, dipropalin, chlomidine, methalpropalin, dinoprop, glyphosate, anilofos, glufosinate ammonium, amiprophos-methyl, sulphosate, piperophos, bialaphos-sodium, bensulide, butamifos, phocarb, 2,4-DEP, H-9201, zytron, imazapyr, imazethapyr, imazaquin, imazamox, imazamox ammonium salt, imazapic, imazamethabenz-methyl, fluroxypyr, fluroxypyr isooctyl ester, clopyralid, picloram, trichlopyr, dithiopyr, haloxydine, 3,5,6-trichloro-2-pyridinol, thiazopyr, fluridone, aminopyralid, diflufenzopyr, triclopyr-butotyl, Clio-dinate, sethoxydim, clethodim, cycloxydim, alloxydim, clefoxydim, butroxydim, tralkoxydim, tepraloxydim, buthidazole, metribuzin, hexazinone, metamitron, ethiozin, ametridione, amibuzin, bromoxynil, bromoxynil octanoate, ioxynil octanoate, ioxynil, dichlobenil, diphenatrile, pyraclonil, chloroxynil, iodobonil, flumetsulam, florasulam, penoxsulam, metosulam, cloransulam-methyl, diclosulam, pyroxsulam, benfuresate, bispyribac-sodium, pyribenzoxim, pyriftalid, pyriminobac-methyl, pyrithiobac-sodium, benzobicylon, mesotrione, sulcotrione, tembotrione, tefuryltrione, bicyclopyrone, ketodpiradox, isoxaflutole, clomazone, fenoxasulfone, methiozolin, fluazolate, pyraflufen-ethyl, pyrazolynate, difenzoquat, pyrazoxyfen, benzofenap, nipyraclofen, pyrasulfotole, topramezone, pyroxasulfone, cafenstrole, flupoxam, aminotriazole, amicarbazone, azafenidin, carfentrazone-ethyl, sulfentrazone, bencarbazone, benzfendizone, butafenacil, bromacil, isocil, lenacil, terbacil, flupropacil, cinidon-ethyl, flumiclorac-pentyl, flumioxazin, S-23121, MK-129, flumezin, pentachlorophenol, dinoseb, dinoterb, dinoterb acetate, dinosam, DNOC, chloronitrophene, medinoterb acetate, dinofenate, oxadiargyl, oxadiazon, pentoxazone, Flufenacet, fluthiacet-methyl, fentrazamide, flufenpyr-ethyl, pyrazon, brompyrazon, metflurazon, kusakira, dimidazon, oxapyrazon, norflurazon, pyridafol, quinclorac, quinmerac, bentazone, pyridate, oxaziclomefone, benazolin, clomazone, cinmethylin, ZJ0702, pyribambenz-propyl, indanofan, sodium chlorate, dalapon, trichloroacetic acid, monochloroacetic acid, hexachloroacetone, flupropanate, cyperquat, bromofenoxim, epronaz, methazole, flurtamone, benfuresate, ethofumesate, tioclorim, chlorthal, fluorochloridone, tavron, acrolein, bentranil, tridiphane, chlorfenpropmethyl, thidiarizonaimin, phenisopham, busoxinone, methoxyphenone, saflufenacil, clacyfos, chloropon, alorac, diethamquat, etnipromid, iprymidam, ipfencarbazone, thiencarbazone-methyl, pyrimisulfan, chlorflurazole, tripropindan, sulglycapin, prosulfalin, cambendichlor, aminocyclopyrachlor, rodethanil, benoxacor, fenclorim, flurazole, fenchlorazole-ethyl, cloquintocet-mexyl, oxabetrinil, MG/91, cyometrinil, DKA-24, mefenpyr-diethyl, furilazole, fluxofenim, isoxadifen-ethyl, dichlormid, halauxifen-methyl, DOW848, UBH-509, D489, LS 82-556, KPP-300, NC-324, NC-330, KH-218, DPX-N8189, SC-0744, DOWCO535, DK-8910, V-53482, PP-600, MBH-001, KIH-9201, ET751, KIH-6127 and KIH-2023.

In one specific embodiment, the active substance (additional herbicides in (iii) above) is selected from one or more of the following compounds: (1) VLCFA inhibitors: acetochlor (CAS No.: 34256-82-1), S-metolachlor (CAS No.: 87392 Dec. 9), pretilachlor (CAS No.: 51218-49-6), butachlor (CAS No.: 23184-66-9), mefenacet (CAS No.: 73250-68-7), anilofos (CAS No.: 64249-01-0), pyroxasulfone (CAS No.: 447399-55-5), flufenacet (CAS No.: 142459-58-3), fentrazamide (CAS No.: 158237 Jul. 1), napropamide (CAS No.: 15299-99-7); (2) Microtubule Assembly inhibitors: pendimethalin (CAS No.: 40487-42-1), butralin (CAS No.: 33629-47-9), trifluralin (CAS No.: 1582 Sep. 8);
(3) PSII inhibitors: metribuzin (CAS No.: 21087-64-9), prometryn (CAS No.: 7287-19-6), atrazine (CAS No.: 1912-24-9), terbuthylazine (CAS No.: 5915-41-3), isoproturon (CAS No.: 34123-59-6), chlorotoluron (CAS No.: 15545-48-9);
(4) PPO inhibitors: flumioxazin (CAS No.: 103361 Sep. 7),

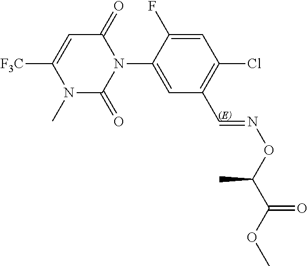 [SMILES: COC(=O)[C@@H](C)O/N=C/c1cc(-n2c(=O)cc(C(F)(F)F)n(C)c2=O)c(F)cc1Cl]

(CAS No.: 2759011-88-4), oxadiazon (CAS No.: 19666-30-9), oxadiargyl (CAS No.: 39807-15-3), sulfentrazone (CAS No.: 122836-35-5), pyraclonil (CAS No.: 158353-15-2), pentoxazone (CAS No.: 110956-75-7), saflufenacil (CAS No.: 372137-35-4), trifludimoxazin (CAS No.: 1258836-72-4), 3-[2-chloro-5-[3,6-dihydro-3-methyl-2,6-dioxo-4-(trifluoromethyl)-1 (2H)-pyrimidinyl]-4-fluorophenyl]-4,5-dihydro-5-methyl-5-isoxazolecarboxylic acid ethyl ester (CAS No.: 1949837-17-5), epyrifenacil (CAS No.: 353292-31-6), tiafenacil (CAS No.: 1220411-29-9);
(5) HPPD inhibitors: isoxaflutole (CAS No.: 141112-29-0), fenpyrazone (CAS No.: 1992017-55-6),

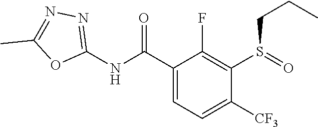 [SMILES: CCC[S@@](=O)c1c(C(F)(F)F)ccc(C(=O)Nc2nnc(C)o2)c1F]

(CAS No.: 2421252-30-2), tefuryltrione (CAS No.: 473278-76-1), benzobicyclon (CAS No.: 156963-66-5), bipyrazone (CAS No.: 1622908-18-2);
(6) DOXP inhibitors:

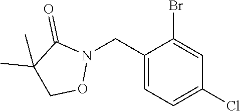 [SMILES: CC1(C)CON(Cc2ccc(Cl)cc2Br)C1=O]

(CAS No.: 2766607-82-1), bixlozone (CAS No.: 81777-95-9);
(7) Synthetic hormones:

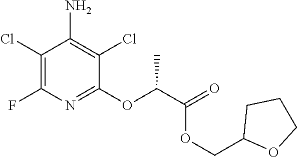 [SMILES: C[C@@H](Oc1nc(F)c(Cl)c(N)c1Cl)C(=O)OCC1CCCO1]

(CAS No.: 2445983-82-2), clopyralid (CAS No.: 1702-17-6), dicamba (CAS No.: 1918-00-9), halauxifen-methyl (CAS No.: 943831-98-9),

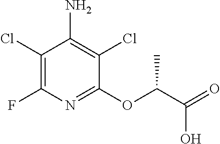 [SMILES: C[C@@H](Oc1nc(F)c(Cl)c(N)c1Cl)C(=O)O]

### (CAS No.: 2445980-81-2), 2,4-D-choline (CAS No.: 1048373-72-3),

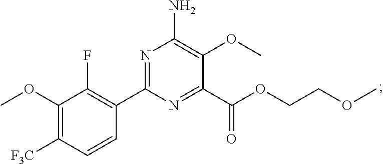

(8) ALS inhibitors: bensulfuron-methyl (CAS No.: 83055-99-6), pyrazosulfuron-ethyl (CAS No.: 93697-74-6), thifensulfuron-methyl (CAS No.: 79277-27-3), metazosulfuron (CAS No.: 868680-84-6), propyrisulfuron (CAS No.: 570415-88-2), thiencarbazone-methyl (CAS No.: 317815-83-1), triafamone (CAS No.: 874195-61-6), pyrimisulfan (CAS No.: 221205-90-9);
(9) FAT inhibitors:

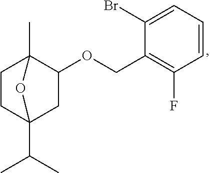 [SMILES: CC(C)C12CCC(C)(O1)C(OCc1c(F)cccc1Br)C2]

cinmethylin (CAS No.: 87818-31-3), oxaziclomefone (CAS No.: 153197-14-9);
(10) Lipid synthesis inhibitors: prosulfocarb (CAS No.: 52888-80-9);
(11) DHODH inhibitors: tetflupyrolimet (CAS No.: 2053901-33-8);
(12) EPSPS inhibitors: glyphosate (CAS No.: 1071-83-6);
(13) GS inhibitors: glufosinate-ammonium (CAS No.: 77182-82-2);
(14) PSI inhibitors: paraquat dichloride (CAS No.: 1910-42-5), diquat dibromide monohydrate (CAS No.: 6385-62-2).

In another specific embodiment, the weight ratio of the effective ingredient (i) and the additional herbicide in (iii) in the composition is 1:100~100:1, 1:80~80:1, 1:50~50:1, 1:30~30:1, 1:20~20:1, 1:10~10:1, 1:5~1:1, or 1:1~5:1.

For use, the commercially available formulations are, if needed, diluted in customary manners, for example using water in the cases of wettable powders, emulsifiable concentrates, dispersions, and water-dispersible granules. For products in the form of dusts, granules for soil application, or solutions for broadcasting and spray, usually no further dilution with inert materials is required prior to use. The required application amount of the compound of Formula I varies with the external conditions such as temperature, humidity, the nature of the used herbicide and the like. It can have a large variation range, for example, between 0.001 and 1.0 kg a.i./ha, or more active substances, but preferably between 0.005 and 750 g a.i./ha, in particular between 0.005 and 250 g a.i./ha.

### DETAILED EMBODIMENTS OF THE INVENTION

The following examples are used to illustrate the present invention and should not be construed as limiting the present invention in any way. The scope of rights claimed by the present invention is described in the claims.

In view of the economy and diversity of the compounds, some compounds were preferably synthesized. Among the many synthesized compounds, selected ones are listed in Table 1 below. The specific compound structure and corresponding compound information are as set forth in Table 1. The compounds in Table 1 are only to better illustrate the present invention, but do not limit the present invention. For a person skilled in the art, it should not be understood that the scope of the above-mentioned subject matters of the present invention is limited to the following compounds.

TABLE 1
Compound structure and ¹H NMR thereof
I

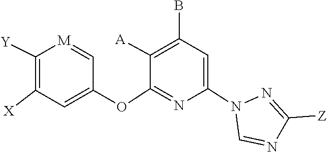

| No. | A | B | X | Y | Z | M | ¹H NMR |
|---|---|---|---|---|---|---|---|
| 1 | CH₃ | CH₃ | CF₃ | H | CF₃ | N | |
| 2 | H | CN | CF₃ | H | CF₃ | CH | ¹H NMR (300 MHz, Chloroform-d) δ |
| | | | | | | | 8.67-8.65(m, 1H), 7.90-7.88 (m, |
| | | | | | | | 1H), 7.63 (d, J = 4.8 Hz, |
| | | | | | | | 2H), 7.51-7.47 (m, 1H), 7.43-7.35 |
| | | | | | | | (m, 1H), 7.31-7.28 (m, 1H). |
| 3 | H | OCH₃ | CF₃ | H | CF₃ | CH | ¹H NMR (300 MHz, Chloroform-d) δ |
| | | | | | | | 8.73 (s, 1H), 7.62-7.51 (m, |
| | | | | | | | 2H), 7.50-7.45 (m, 1H), 7.43-7.33 |
| | | | | | | | (m, 1H), 7.25-7.22 (m, 1H), 6.48- |
| | | | | | | | 6.45 (m, 1H), 3.98 (s, 3H). |
| 4 | CH₃ | CH₃ | CF₃ | H | CF₃ | CH | |
| 5 | H | CH₃ | CF₃ | H | CF₃ | CH | ¹H NMR (300 MHz, Chloroform-d) δ |
| | | | | | | | 8.73 (s, 1H), 7.62-7.44 |
| | | | | | | | (m, 4H), 7.40-7.33(m, |
| | | | | | | | 1H), 6.82 (br, 1H), 2.49 (s, 3H). |
| 6 | H | CH₃ | CF₃ | H | CF₃ | N | ¹H NMR (300 MHz, Chloroform-d) δ |
| | | | | | | | 8.93-8.70 (m, 2H), 8.62 (s, 1H), |
| | | | | | | | 7.83 (s, 1H), 7.57 (s, 1H), |
| | | | | | | | 6.93 (s, 1H), 2.53 (s, 3H). |
| 7 | Cl | H | CF₃ | H | CF₃ | CH | ¹H NMR (300 MHz, Chloroform-d) δ |
| | | | | | | | 8.56 (s, 1H), 8.01 (d, J = 8.1 |
| | | | | | | | Hz, 1H), 7.67-7.58 (m, 3H), |
| | | | | | | | 7.55-7.50 (m, 1H), 7.45-7.38 (m, 1H). |
| 8 | CH₃ | H | CF₃ | H | CF₃ | CH | ¹H NMR (300 MHz, Chloroform-d) δ |
| | | | | | | | 8.59 (s, 1H), 7.81-7.73 |
| | | | | | | | (m, 1H), 7.61-7.45 (m, 4H), |
| | | | | | | | 7.41-7.33 (m, 1H), 2.44 (s, 3H). |
| 9 | CH₃ | H | OCF₃ | H | CF₃ | CH | ¹H NMR (300 MHz, Chloroform-d) δ |
| | | | | | | | 8.62 (s, 1H), 7.82-7.72 (m, 1H), 7.55 |
| | | | | | | | (d, J = 7.8 Hz, 1H), 7.50-7.41 (m, 1H), |
| | | | | | | | 7.20-7.09 (m, 3H), 2.42 (s, 3H). |
| 10 | CH₃ | H | CF₃ | F | CF₃ | CH | ¹H NMR (300 MHz, Chloroform-d) δ |
| | | | | | | | 8.58 (s, 1H), 7.80-7.75 (m, |
| | | | | | | | 1H), 7.56 (d, J = 7.8 Hz, 1H), |
| | | | | | | | 7.51-7.28 (m, 3H), 2.42 (s, 3H). |
| 11 | CH₃ | H | CF₃ | H | CF₃ | N | ¹H NMR (300 MHz, Chloroform-d) δ |
| | | | | | | | 8.85-8.71 (m, 2H), 8.58 (s, 1H), |
| | | | | | | | 7.89-7.78 (m, 2H), 7.63 |
| | | | | | | | (d, J = 7.8 Hz, 1H), 2.46 (s, 3H). |
| 12 | CH₃ | H | CHF₂ | H | CF₃ | CH | ¹H NMR (300 MHz, Chloroform-d) δ |
| | | | | | | | 8.82-8.72 (m, 2H), 7.98-7.96 |
| | | | | | | | (m, 1H), 7.90-7.83 (m, 1H), |
| | | | | | | | 7.76-7.71 (m, 1H), 7.64 (d, |
| | | | | | | | J = 7.8 Hz, 1H), 6.82 |
| | | | | | | | (d, J = 54.0 Hz, 1H), 6.59-6.51 |
| | | | | | | | (m, 1H), 2.42 (s, 3H). |
| 13 | CH₃ | H | CF₃ | H | 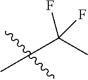 [SMILES: *C(C)(F)F] | CH | ¹H NMR (300 MHz, Chloroform-d) δ 8.55 (s, 1H), 7.79-7.73 (m, 1H), 7.59-7.48 (m, 4H), 7.41-7.34 (m, 1H), 2.43 (s, 3H), 2.15-2.02 (m, 3H). |
| 14 | CH₃ | H | CF₃ | H | 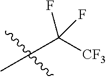 [SMILES: *C(F)(F)C(F)(F)F] | CH | ¹H NMR (300 MHz, Chloroform-d) δ 8.62 (s, 1H), 7.81-7.75 (m, 1H), 7.65-7.44 (m, 4H), 7.41-7.34 (m, 1H), 2.44 (s, 3H). |
| 15 | CH₃ | H | CF₃ | H | 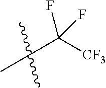 [SMILES: *C(F)(F)C(F)(F)F] | N | ¹H NMR (300 MHz, Chloroform-d) δ 8.81 (s, 1H), 8.75 16(d, J = 2.4 Hz, 1H), 8.62-8.60 (m, 1H), 7.87-7.79 (m, 2H), 7.64 (d, J = 7.8 Hz, 1H), 2.46 (s, 3H). |
| 16 | CH₂CH₃ | H | CF₃ | H | CF₃ | CH | ¹H NMR (300 MHz, Chloroform-d) δ |
| | | | | | | | 8.58 (s, 1H), 7.84-7.74 (m, |
| | | | | | | | 1H), 7.63-7.51 (m, 3H), 7.50-7.45 (m, |
| | | | | | | | 1H), 7.41-7.32 (m, 1H), 2.84 (q, |
| | | | | | | | J = 7.5 Hz, 2H), 1.35 (t, J = 7.5 Hz, 3H). |
| 17 | CH₂CH₃ | H | CF₃ | H | CF₃ | N | ¹H NMR (300 MHz, Chloroform-d) δ |
| | | | | | | | 8.81 (s, 1H), 8.75 (s, 1H), 8.58 |
| | | | | | | | (s, 1H), 7.85 (m, 2H), 7.65 (d, J = 7.8 |
| | | | | | | | Hz, 1H), 2.86 (q, J = 7.5 Hz, 2H), |
| | | | | | | | 1.36 (t, J = 7.5 Hz, 3H). |
| 18 | CH₂CH₃ | H | CF₃ | H | 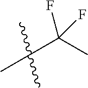 [SMILES: *C(C)(F)F] | CH | ¹H NMR (300 MHz, Chloroform-d) δ 8.54 (s, 1H), 7.77 (d, J = 7.8 Hz, 1H), 7.58-7.46 (m, 4H), 7.38-7.34 (m, 1H), 2.86 (q, J = 7.5 Hz, 2H), 2.08 (t, J = 18.3 Hz, 3H), 1.34 (t, J = 7.5 Hz, 3H). |
| 19 | CH₂CH₃ | H | CF₃ | H | 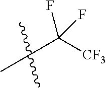 [SMILES: *C(F)(F)C(F)(F)F] | CH | ¹H NMR (300 MHz, Chloroform-d) δ 8.62 (s, 1H), 7.80 (d, J = 7.8 Hz, 1H), 7.62-7.48 (m, 4H), 7.38-7.33 (m, 1H), 2.86 (q, J = 7.5 Hz, 2H), 1.36 (t, J = 7.5 Hz, 3H). |
| 20 | CH₂CH₃ | H | 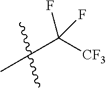 [SMILES: *C(F)(F)C(F)(F)F] | H | CF₃ | CH | ¹H NMR (300 MHz, Chloroform-d) δ 8.53 (s, 1H), 7.80 (d, J = 7.5 Hz, 1H), 7.62-7.48 (m, 4H), 7.38-7.33 (m, 1H), 2.86 (q, J = 7.5 Hz, 2H), 1.36 (t, J = 7.5 Hz, 3H). |
| 21 | 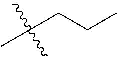 [SMILES: *CCC] | H | CF₃ | H | CF₃ | CH | ¹H NMR (300 MHz, Chloroform-d) δ 8.63 (s, 1H), 7.82 (d, J = 7.8 Hz, 1H), 7.67-7.56 (m, 3H), 7.55-7.49 (m, 1H), 7.45- |
| | | | | | | | 7.37 (m, 1H), 2.82 (t, |
| | | | | | | | J = 7.5 Hz, 2H), 1.90-1.74 (m, 2H), |
| | | | | | | | 1.09 (t, J = 7.5 Hz, 3H). |
| 22 | 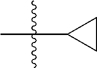 [SMILES: *C1CC1] | H | CF₃ | H | CF₃ | CH | ¹H NMR (300 MHz, Chloroform-d) δ 8.58 (s, 1H), 7.58-7.44 (m, 5H), 7.42-7.35 (m, 1H), 2.35-2.20 (m, 1H), 1.16-1.08 (m, 2H), 0.86-0.78 (m, 2H). |
| 23 | 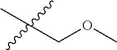 [SMILES: *COC] | H | CF₃ | H | CF₃ | CH | ¹H NMR (300 MHz, Chloroform-d) δ 8.62 (s, 1H), 8.09 (d, J = 7.8 Hz, 1H), 7.69 (d, J = 7.8 Hz, 1H), 7.63- |
| | | | | | | | 7.56 (m, 2H), 7.54-7.49 (m, 1H), |
| | | | | | | | 7.43-7.37 (m, 1H), 4.69 (s, 2H), |
| | | | | | | | 3.57 (s, 3H). |
| 24 | F | H | CF₃ | H | CF₃ | CH | |
| 25 | CN | H | CF₃ | H | CF₃ | CH | |
| 26 | OCH₃ | H | CF₃ | H | CF₃ | CH | |
| 27 | OCH₂CH₃ | H | CF₃ | H | CF₃ | CH | |
| 28 | 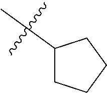 [SMILES: *C1CCCC1] | H | CF₃ | H | CF₃ | CH | |
| 29 | 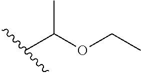 [SMILES: *C(C)OCC] | H | CF₃ | H | CF₃ | CH | |
| 30 | CH₃ | F | CF₃ | H | CF₃ | CH | |
| 31 | CH₃ | Cl | CF₃ | H | CF₃ | CH | |
| 32 | CH₃ | CH₂CH₃ | CF₃ | H | CF₃ | CH | |
| 33 | CH₃ | 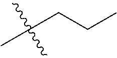 [SMILES: *CCC] | CF₃ | H | CF₃ | CH | |
| 34 | CH₃ | 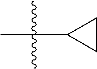 [SMILES: *C1CC1] | CF₃ | H | CF₃ | CH | |
| 35 | CH₃ | 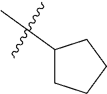 [SMILES: *C1CCCC1] | CF₃ | H | CF₃ | CH | |
| 36 | CH₃ | 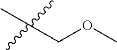 [SMILES: *COC] | CF₃ | H | CF₃ | CH | |
| 37 | CH₃ | 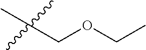 [SMILES: *COCC] | CF₃ | H | CF₃ | CH | |
| 38 | CH₃ | 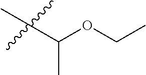 [SMILES: *C(C)OCC] | CF₃ | H | CF₃ | CH | |
| 39 | CH₃ | H | F | H | CF₃ | CH | ¹H NMR (300 MHz, Chloroform-d) δ |
| | | | | | | | 8.65 (s, 1H), 7.75 (d, J = 7.8 Hz, |
| | | | | | | | 1H), 7.54 (d, J = 7.8 Hz, 1H), 7.44- |
| | | | | | | | 7.32 (m, 1H), 7.08-6.80 (m, 3H), |
| | | | | | | | 2.41 (s, 3H). |
| 40 | CH₃ | H | Cl | H | CF₃ | CH | ¹H NMR (300 MHz, Chloroform-d) δ |
| | | | | | | | 8.65 (s, 1H), 7.75 (d, J = 7.8 Hz, |
| | | | | | | | 1H), 7.53 (d, J = 7.8 Hz, 1H), 7.36 (t, |
| | | | | | | | J = 8.1 Hz, 1H), 7.29-7.15 (m, |
| | | | | | | | 2H), 7.11-7.01 (m, 1H), 2.41 (s, 3H). |
| 41 | CH₃ | H | CH₃ | H | CF₃ | CH | ¹H NMR (300 MHz, Chloroform-d) δ |
| | | | | | | | 8.64 (s, 1H), 7.71 (d, J = 7.8 |
| | | | | | | | Hz, 1H), 7.48 (d, J = 7.8 |
| | | | | | | | Hz, 1H), 7.35-7.24 (m, 1H), 7.06 (d, |
| | | | | | | | J = 7.5 Hz, 1H), 7.01-6.91 (m, |
| | | | | | | | 2H), 2.41 (s, 3H), 2.39 (s, 3H). |
| 42 | CH₃ | H | CH₂CH₃ | H | CF₃ | CH | |
| 43 | CH₃ | H | OCHF₂ | H | CF₃ | CH | ¹H NMR (300 MHz, Chloroform-d) δ |
| | | | | | | | 8.65 (s, 1H), 7.75 (d, J = 7.8 |
| | | | | | | | Hz, 1H), 7.53 (d, J = 7.8 Hz, 1H), 7.46- |
| | | | | | | | 7.36 (m, 1H), 7.10-6.96 (m, 3H), |
| | | | | | | | 6.55 (t, J = 75.0 Hz, 1H), 2.41 (s, 3H). |
| 44 | CH₃ | H | OCF₃ | H | CF₃ | N | |
| 45 | CH₃ | H | CF₃ | Cl | CF₃ | CH | |
| 46 | CH₃ | H | CF₃ | Br | CF₃ | CH | |
| 47 | CH₃ | H | CF₃ | H | H | CH | |
| 48 | CH₃ | H | CF₃ | H | F | CH | |
| 49 | CH₃ | H | CF₃ | H | Cl | CH | |
| 50 | CH₃ | H | CF₃ | H | CN | CH | |
| 51 | CH₃ | H | CF₃ | H | CH₃ | CH | |
| 52 | CH₃ | H | CF₃ | H | CH₂CH₃ | CH | |
| 53 | CH₃ | H | CF₃ | H | 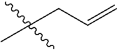 [SMILES: *CC=C] | CH | |
| 54 | CH₃ | H | CF₃ | H | 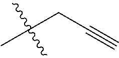 [SMILES: *CC#C] | CH | |
| 55 | CH₃ | H | CF₃ | H | 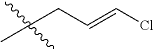 [SMILES: *C/C=C/Cl] | CH | |
| 56 | CH₃ | H | CF₃ | H | 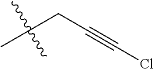 [SMILES: *CC#CCl] | CH | |
| 57 | CH₃ | H | CF₃ | H | OCH₃ | CH | |
| 58 | CH₃ | H | CF₃ | H | OCF₃ | CH | |
| 59 | CH₃ | H | CF₃ | H | OCHF₂ | CH | |
| 60 | CH₃ | H | CF₃ | H | 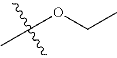 [SMILES: *OCC] | CH | |
| 61 | CH₃ | H | CF₃ | H | 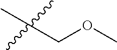 [SMILES: *COC] | CH | |
| 62 | CH₃ | H | CF₃ | H | SO₂CH₃ | CH | |
| 63 | CH₃ | H | CF₃ | H | 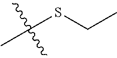 [SMILES: *SCC] | CH | |
| 64 | CH₃ | H | CF₃ | H | 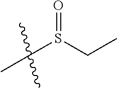 [SMILES: *S(=O)CC] | CH | |
| 65 | CH₃ | H | OCF₃ | H | 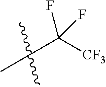 [SMILES: *C(F)(F)C(F)(F)F] | CH | ¹H NMR (300 MHz, Chloroform-d) δ 8.65 (s, 1H), 7.80-7.74 (m, 1H), 7.57 (d, J = 7.8 Hz, 1H), 7.49-7.41 (m, 1H), 7.16-7.09 (m, 3H), 2.42 (s, 3H). |
| 66 | CH₃ | H | OCF₃ | F | CF₃ | CH | ¹H NMR (300 MHz, Chloroform-d) δ |
| | | | | | | | 8.62 (s, 1H), 7.77 (d, J = 7.8 |
| | | | | | | | Hz, 1H), 7.57 (d, J = 7.8 |
| | | | | | | | Hz, 1H), 7.34-7.19 (m, 2H), 7.17- |
| | | | | | | | 7.05 (m, 1H), 2.43 (s, 3H). |
| 67 | H | OCH₃ | OCF₃ | H | CHF₂ | CH | ¹H NMR (300 MHz, Chloroform-d) δ |
| | | | | | | | 8.74 (s, 1H), 7.43 (t, J = 8.1 |
| | | | | | | | Hz, 1H), 7.19 (d, J = 1.8 Hz, |
| | | | | | | | 1H), 7.15-7.04 (m, 3H), 6.75 |
| | | | | | | | (t, J = 53.4 Hz, 1H), 6.41 (d, J = |
| | | | | | | | 1.8 Hz, 1H), 3.94 (s, 3H). |
| 68 | H | 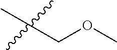 [SMILES: *COC] | CF₃ | H | CF₃ | CH | ¹H NMR (300 MHz, DMSO-d6) δ 9.13 (s, 1H), 7.98 (s, 1H), 7.73- 7.54 (m, 4H), 7.41-7.35 (m, 1H), 5.73 (s, 2H), 3.37 (s, 3H). |
| 69 | CH₂CH₃ | H | CF₂CH₃ | H | CF₃ | CH | ¹H NMR (300 MHz, Chloroform-d) δ |
| | | | | | | | 8.58 (s, 1H), 7.85 (d, J = 7.8 |
| | | | | | | | Hz, 1H), 7.82-7.74 (m, 2H), |
| | | | | | | | 7.60-7.50 (m, 2H), 7.39 (d, J = |
| | | | | | | | 8.1 Hz, 1H), 2.84 (q, J = 7.5 Hz, |
| | | | | | | | 2H), 2.64 (s, 3H), 1.35 (t, J = 7.5 |
| | | | | | | | Hz, 3H). |
| 70 | CH₃ | H | CF₃ | F | CHF₂ | CH | ¹H NMR (300 MHz, Chloroform-d) δ |
| | | | | | | | 8.58 (s, 1H), 7.77 (d, J = 7.8 |
| | | | | | | | Hz, 1H), 7.54 (d, J = 7.8 |
| | | | | | | | Hz, 1H), 7.50-7.44 (m, 1H), |
| | | | | | | | 7.42-7.34 (m, 1H), 7.33-7.23 (m, |
| | | | | | | | 1H), 6.75 (t, J = 53.4 Hz, 1H), |
| | | | | | | | 2.43 (s, 3H). |
| 71 | CH₂CH₃ | H | OCF₃ | H | CF₃ | CH | ¹H NMR (300 MHz, Chloroform-d) δ |
| | | | | | | | 8.63 (s, 1H), 7.79 (d, J = |
| | | | | | | | 7.8 Hz, 1H), 7.59 (d, J = 7.8 |
| | | | | | | | Hz, 1H), 7.46 (t, J = 8.1 Hz, |
| | | | | | | | 1H), 7.20-7.06 (m, 3H), 2.83 (q, J = |
| | | | | | | | 3 7.5 Hz, 2H), 1.34 (t, J = 7.5 Hz, |
| | | | | | | | 3H). |
| 72 | CH₂CH₃ | H | CF₃ | H | CHF₂ | CH | ¹H NMR (300 MHz, Chloroform-d) δ |
| | | | | | | | 8.58 (s, 1H), 7.78 (d, J = 7.8 |
| | | | | | | | Hz, 1H), 7.64-7.42 (m, 4H), |
| | | | | | | | 7.37 (d, J = 7.2 Hz, 1H), 6.75 |
| | | | | | | | (t, J = 53.4 Hz, 1H), 2.83 (q, J = 7.5 |
| | | | | | | | Hz, 2H), 1.35 (t, J = 7.5 Hz, |
| | | | | | | | 3H). |
| 73 | CH₂CH₃ | H | OCF₃ | H | CHF₂ | CH | ¹H NMR (300 MHz, Chloroform-d) δ |
| | | | | | | | 8.62 (s, 1H), 7.77 (d, J = 7.8 |
| | | | | | | | Hz, 1H), 7.56 (d, J = 7.8 |
| | | | | | | | Hz, 1H), 7.45 (t, J = 8.1 Hz, |
| | | | | | | | 1H), 7.20-7.04 (m, 3H), 6.75 (t, J = |
| | | | | | | | 53.4 Hz, 1H), 2.82 (q, J = 7.5 |
| | | | | | | | Hz, 2H), 1.33 (t, J = 7.5 Hz, 3H). |
| 74 | CH₂CH₃ | H | CF₃ | H | CHF₂ | N | ¹H NMR (300 MHz, Chloroform-d) δ |
| | | | | | | | 8.81(s, 1H), 8.76 (s, 1H), |
| | | | | | | | 8.59 (s, 1H), 7.91-7.79 (m, |
| | | | | | | | 2H), 7.64 (d, J = 7.8 Hz, 1H), |
| | | | | | | | 6.75 (t, J = 53.4 Hz, 1H), 2.86 |
| | | | | | | | (q, J = 7.5 Hz, 2H), 1.37 (t, J = 7.5 |
| | | | | | | | Hz, 3H). |
| 75 | CH₃ | H | OCHF₂ | F | CF₃ | CH | ¹H NMR (300 MHz, Chloroform-d) δ |
| | | | | | | | 8.66 (s,1 H), 7.76 (d, J = 7.8 |
| | | | | | | | Hz, 1H), 7.55 (d, J = 7.8 Hz, 1H), |
| | | | | | | | 7.27-7.13 (m, 2H), 7.10-6.96 (m, 1H), |
| | | | | | | | 6.62 (t, J = 75.0 Hz, 1H), 2.43 (s, 3H). |
| 76 | H | OCH₃ | CF₃ | F | CF₃ | CH | ¹H NMR (300 MHz, Chloroform-d) δ |
| | | | | | | | 8.71 (s, 1H), 7.49-7.42 (m, |
| | | | | | | | 1H), 7.40-7.33 (m, 1H), |
| | | | | | | | 7.29-7.20 (m, 2H), 6.46 (d, J = 1.8 |
| | | | | | | | Hz, 1H), 3.99 (s, 3H). |
| 77 | 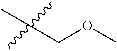 [SMILES: *COC] | H | OCF₃ | H | 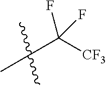 [SMILES: *C(F)(F)C(F)(F)F] | CH | ¹H NMR (300 MHz, Chloroform-d) δ 8.67 (s, 1H), 8.06 (d, J = 7.8 Hz, 1H), 7.67 (d, J = 7.8 Hz, 1H), 7.46 (t, J = 8.1 Hz, 1H), 7.17-7.09 (m, 2H), 6.82-6.68 (m, 1H), 4.66 (s, 2H), 3.55 (s, 3H). |
| 78 | H | OCH₃ | OCF₃ | H | CF₃ | CH | ¹H NMR (300 MHz, Chloroform-d) δ |
| | | | | | | | 8.77 (s, 1H), 7.44 (t, J = 9.0 |
| | | | | | | | Hz, 1H), 7.23 (d, J = 1.8 Hz, |
| | | | | | | | 1H), 7.19-7.04 (m, 3H), 6.45 (d, J = |
| | | | | | | | 1.8 Hz, 1H), 3.98 (s, 3H). |
| 79 | CH₃ | H | OCHF₂ | H | CHF₂ | CH | ¹H NMR (300 MHz, Chloroform-d) δ |
| | | | | | | | 8.65 (s, 1H), 7.75 (d, J = 7.8 |
| | | | | | | | Hz, 1H), 7.52 (d, J = 7.8 |
| | | | | | | | Hz, 1H), 7.41 (t, J = 8.1 Hz, 1H), |
| | | | | | | | 7.13-6.97 (m, 3H), 6.55 (t, J = |
| | | | | | | | 53.4 Hz, 1H), 2.41 (s, 3H). |
| 80 | CH₃ | H | OCF₃ | F | CHF₂ | CH | ¹H NMR (300 MHz, Chloroform-d) δ |
| | | | | | | | 8.60 (s, 1H), 7.75 (d, J = 7.8 |
| | | | | | | | Hz, 1H), 7.52 (d, J = 7.8 |
| | | | | | | | Hz, 1H), 7.34-7.06 (m, 3H), 6.74 (t, |
| | | | | | | | J = 53.4 Hz, 1H), 2.41 (s, 3H). |
| 81 | CH₃ | H | F | H | CHF₂ | CH | ¹H NMR (300 MHz, Chloroform-d) δ |
| | | | | | | | 8.56 (s, 1H), 7.73 (d, J = |
| | | | | | | | 7.8 Hz, 1H), 7.49 (d, J = 7.8 |
| | | | | | | | Hz, 1H), 7.34-7.06 (m, 4H), 6.74 (t, |
| | | | | | | | J = 53.4 Hz, 1H), 2.41 (s, 3H) |
| 82 | CH₃ | H | CH₃ | H | CHF₂ | CH | ¹H NMR (300 MHz, Chloroform-d) δ |
| | | | | | | | 8.65 (s, 1H), 7.70 (d, J = 7.8 |
| | | | | | | | Hz, 1H), 7.47 (d, J = 7.8 |
| | | | | | | | Hz, 1H), 7.36-7.23 (m, 1H), |
| | | | | | | | 7.11-7.02 (m, 1H), |
| | | | | | | | 7.00-6.90 (m, 2H), 6.78-6.60 (m, 1H), |
| | | | | | | | 2.40 (s, 3H), 2.39 (s, 3H). |
| 83 | CH₃ | H | OCF₃ | H | CHF₂ | CH | ¹H NMR (300 MHz, Chloroform-d) δ |
| | | | | | | | 8.62 (s, 1H), 7.76 (d, J = 7.8 |
| | | | | | | | Hz, 1H), 7.54 (d, J = 7.8 |
| | | | | | | | Hz, 1H), 7.46 (t, J = 8.1 Hz, 1H), |
| | | | | | | | 7.29-7.09 (m, 3H), 6.75 (t, J = |
| | | | | | | | 53.4 Hz, 1H), 2.42 (s, 3H). |
| 84 | CH₃ | H | CHF₂ | H | CHF₂ | CH | ¹H NMR (300 MHz, Chloroform-d) δ |
| | | | | | | | 8.59 (s, 1H), 7.74 (d, J = 7.8 |
| | | | | | | | Hz, 1H), 7.59-7.46 (m, 2H), |
| | | | | | | | 7.47-7.21 (m, 3H), 6.95-6.44 (m, |
| | | | | | | | 2H), 2.41 (s, 3H). |
| 85 | CH₂CH₃ | H | CH₂F | H | CF₃ | CH | ¹H NMR (300 MHz, Chloroform-d) δ |
| | | | | | | | 8.62 (s, 1H), 7.76 (d, J = 7.8 |
| | | | | | | | Hz, 1H), 7.53 (d, J = 7.8 |
| | | | | | | | Hz, 1H), 7.50-7.49 (m, 1H), |
| | | | | | | | 7.24 (d, J = 8.7 Hz, 2H), 7.16 (d, J = |
| | | | | | | | 8.1 Hz, 1H), 5.41 (d, J = 48.0 |
| | | | | | | | Hz, 2H), 2.83 (d, J = 7.5 Hz, 2H), 1.34 |
| | | | | | | | (t, J = 7.5 Hz, 3H). |
| 86 | H | SCH₃ | CF₃ | H | CF₃ | CH | ¹H NMR (300 MHz, Chloroform-d) δ |
| | | | | | | | 8.70 (s, 1H), 7.61-7.42 (m, |
| | | | | | | | 4H), 7.40-7.31 (m, 1H), |
| | | | | | | | 6.77 (s, 1H), 2.59 (s, 3H). |
| 87 | H | SCH₃ | OCF₃ | H | CF₃ | CH | ¹H NMR (300 MHz, Chloroform-d) δ |
| | | | | | | | 8.74 (s, 1H), 7.50-7.40 (m, |
| | | | | | | | 2H), 7.18-7.04 (m, 3H), |
| | | | | | | | 6.75 (d, J = 1.5 Hz, 1H), 2.58 (s, 3H). |
| 88 | CH₃ | H | CN | H | CF₃ | CH | ¹H NMR (300 MHz, Chloroform-d) δ |
| | | | | | | | 8.59 (s, 1H), 7.79 (d, J = 7.8 |
| | | | | | | | Hz, 1H), 7.61-7.54 (m, 3H), |
| | | | | | | | 7.49-7.40 (m, 2H), 2.43 (s, 3H). |
| 89 | CH₂CH₃ | H | CN | H | CF₃ | CH | ¹H NMR (300 MHz, Chloroform-d) δ |
| | | | | | | | 8.58 (s, 1H), 7.80 (d, J = |
| | | | | | | | 7.8 Hz, 1H), 7.62-7.51 (m, 3H), |
| | | | | | | | 7.49-7.39 (m, 2H), 2.82 (q, J = 7.5 Hz, |
| | | | | | | | 2H), 1.33 (t, J = 7.5 Hz, 3H). |
| 90 | 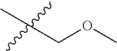 [SMILES: *COC] | H | CN | H | CF₃ | CH | ¹H NMR (300 MHz, Chloroform-d) δ 8.60 (s, 1H), 8.08 (d, J = 7.8 Hz, 1H), 7.69 (d, J = 7.8 Hz, 1H), 7.64-7.52 (m, 2H), 7.51-7.40 (m, 2H), 4.65 (s, 2H), 3.54 (s, 3H). |
| 91 | 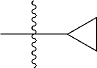 [SMILES: *C1CC1] | H | CN | H | CF₃ | CH | ¹H NMR (300 MHz, Chloroform-d) δ 8.60-8.57 (m, 1H), 7.59-7.53 (m, 3H), 7.50-7.46 (m, 2H), 7.46 (s, 1H), 2.30-2.17 (m, 1H), 1.15- 1.08 (m, 2H), 0.85-0.80 (m, 2H). |
| 92 | H | CH₃ | CN | H | CF₃ | CH | ¹H NMR (300 MHz, Chloroform-d) δ |
| | | | | | | | 8.72 (s, 1H), 7.60-7.51 (m, |
| | | | | | | | 3H), 7.48-7.40 (m, 2H), |
| | | | | | | | 6.86-6.82 (m, 1H), 2.51-2.48 (m, 3H). |
| 93 | H | OCH₃ | CN | H | CF₃ | CH | ¹H NMR (300 MHz, Chloroform-d) δ |
| | | | | | | | 8.61 (s, 1H), 7.51-7.40 (m, |
| | | | | | | | 2H), 7.38-7.29 (m, 2H), 7.14 |
| | | | | | | | (s, 1H), 6.37 (s, 1H), 3.88 (s, 3H). |
| 94 | CH₂CH₃ | H | CN | H | CF₃ | N | ¹H NMR (300 MHz, Chloroform-d) δ |
| | | | | | | | 8.86-8.72 (m, 2H), 8.60 (s, |
| | | | | | | | 1H), 7.91-7.75 (m, 2H), 7.69 |
| | | | | | | | (d, J = 7.8 Hz, 1H), 2.85 (d, J = 7.5 Hz, |
| | | | | | | | 2H), 1.35 (t, J = 7.5 Hz, 3H). |
| 95 | H | CH₃ | CN | H | CF₃ | N | ¹H NMR (300 MHz, Chloroform-d) δ |
| | | | | | | | 8.81-8.75 (m, 2H), 8.69 (s, |
| | | | | | | | 1H), 7.83-7.79 (m, 1H), 7.59 |
| | | | | | | | (s, 1H), 6.94 (s, 1H), 2.53 (s, 3H). |
| 96 | CH₂CH₃ | H | CN | F | CF₃ | CH | ¹H NMR (300 MHz, Chloroform-d) δ |
| | | | | | | | 8.61 (s, 1H), 7.81 (d, J = 7.8 |
| | | | | | | | Hz, 1H), 7.60 (d, J = 7.8 |
| | | | | | | | Hz, 1H), 7.52-7.36 (m, 2H), |
| | | | | | | | 7.36-7.28 (m, 1H), 2.82 (q, J = |
| | | | | | | | 7.5 Hz, 2H), 1.33 (t, J = 7.5 Hz, 3H). |
| 97 | CH₂CH₃ | H | CF₃ | H | OCF₃ | CH | |
| 98 | CH₃ | H | CF₃ | H | 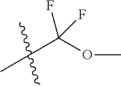 [SMILES: *C(F)(F)OC] | CH | |
| 99 | CH₂CH₃ | H | CF₃ | H | 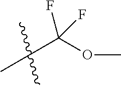 [SMILES: *C(F)(F)OC] | CH | |
| 100 | CH₃ | H | CF₃ | H | 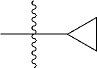 [SMILES: *C1CC1] | CH | ¹H NMR (300 MHz, Chloroform-d) δ 8.38 (s, 1H), 7.74-7.66 (m, 1H), 7.54-7.47 (m, 4H), 7.37-7.31 (m, 1H), 2.39 (s, 3H), 2.16- 2.00 (m, 1H), 1.04-0.93 (m, 4H). |
| 101 | H | OCH₃ | CHF₂ | H | CF₃ | CH | ¹H NMR (300 MHz, Chloroform-d) δ |
| | | | | | | | 8.75 (s, 1H), 7.52 (t, J = 8.1 |
| | | | | | | | Hz, 1H), 7.45-7.26 (m, 3H), |
| | | | | | | | 7.21 (d, J = 1.8 Hz, 1H), 6.67 |
| | | | | | | | (t, J = 56.4 Hz, 1H), 6.43 (d, J = |
| | | | | | | | 1.8 Hz, 1H), 3.96 (s, 3H). |
| 102 | H | OCH₃ | F | H | CF₃ | CH | ¹H NMR (300 MHz, Chloroform-d) δ |
| | | | | | | | 8.82 (s, 1H), 7.46-7.32 (m, |
| | | | | | | | 1H), 7.23-7.18 (m, 1H), |
| | | | | | | | 7.03-6.86 (m, 3H), 6.41 (s, 1H), |
| | | | | | | | 3.96 (s, 3H). |
| 103 | H | OCH₃ | CH₃ | H | CF₃ | CH | ¹H NMR (300 MHz, Chloroform-d) δ |
| | | | | | | | 8.87 (s, 1H), 7.36-7.28 (m, |
| | | | | | | | 1H), 7.17 (d, J = 1.8 Hz, 1H), |
| | | | | | | | 7.07 (d, J = 7.5 Hz, 1H), 7.01- |
| | | | | | | | 6.91 (m, 2H), 6.33 (d, J = 1.8 |
| | | | | | | | Hz, 1H), 3.93 (s, 3H), 2.38 (s, 3H). |
| 104 | H | OCH₃ | OCHF₂ | H | CF₃ | CH | ¹H NMR (300 MHz, Chloroform-d) δ |
| | | | | | | | 8.80 (s, 1H), 7.41 (t, J = 8.1 |
| | | | | | | | Hz, 1H), 7.21 (d, J = 1.8 Hz, |
| | | | | | | | 1H), 7.10-6.96 (m, 3H), 6.83- |
| | | | | | | | 6.26 (m, 2H), 3.96 (s, 3H). |
| 105 | H | OCH₃ | OCF₃ | F | CF₃ | CH | ¹H NMR (300 MHz, Chloroform-d) δ |
| | | | | | | | 8.73 (s, 1H), 7.31-7.20 (m, |
| | | | | | | | 3H), 7.12 (d, J = 1.8 Hz, 1H), |
| | | | | | | | 6.44 (d, J = 1.8 Hz, 1H), 3.97 (s, 3H). |
| 106 | H | OCH₃ | CF₃ | H | CF₃ | N | ¹H NMR (300 MHz, Chloroform-d) δ |
| | | | | | | | 8.80 (s, 1H), 8.73 (s, 1H), |
| | | | | | | | 8.66 (s, 1H), 7.82 (s, 1H), 7.28 |
| | | | | | | | (d, J = 1.8 Hz, 1H), 6.55 (d, J = |
| | | | | | | | 1.8 Hz, 1H), 4.00 (s, 3H). |
| 107 | H | OCH₂CH₃ | CF₃ | H | CF₃ | N | ¹H NMR (300 MHz, Chloroform-d) δ |
| | | | | | | | 8.80 (s, 1H), 8.79 (s, 1H), 8.66 |
| | | | | | | | (s, 1H), 7.82 (s, 1H), 7.28 |
| | | | | | | | (d, J = 2.1 Hz, 1H),6.53 (d, J = 2.1 |
| | | | | | | | Hz, 1H), 4.24 (q, J = 7.2 |
| | | | | | | | Hz, 2H), 1.50 (t, J = 7.2 Hz, 3H). |
| 108 | H | OCH₂CH₃ | OCF₃ | H | CF₃ | CH | ¹H NMR (300 MHz, Chloroform-d) δ |
| | | | | | | | 8.77 (s, 1H), 7.51-7.38 |
| | | | | | | | (m, 1H), 7.25-7.06 (m, 4H), |
| | | | | | | | 6.43-6.39 (m, 1H), 4.20 (q, J = 7.2 Hz, |
| | | | | | | | 2H), 1.48 (t, J = 7.2 Hz, 3H). |
| 109 | H | OCH₂CH₃ | CF₃ | H | CF₃ | CH | ¹H NMR (300 MHz, Chloroform-d) δ |
| | | | | | | | 8.72 (s, 1H), 7.61-7.42 (m, |
| | | | | | | | 3H), 7.41-7.30 (m, 1H), |
| | | | | | | | 7.25-7.14 (m, 1H), 6.43-6.40 |
| | | | | | | | (m, 1H), 4.20 (q, J = 7.2 Hz, |
| | | | | | | | 2H), 1.49 (t, J = 7.2 Hz, 3H). |
| 110 | H | OCH₂CH₃ | CF₃ | F | CF₃ | CH | ¹H NMR (300 MHz, Chloroform-d) δ |
| | | | | | | | 8.77 (s, 1H), 7.48-7.41 (m, |
| | | | | | | | 1H), 7.25-7.06 (m, 3H), |
| | | | | | | | 6.42-6.39 (m, 1H), 4.20 (q, J = 7.2 Hz, |
| | | | | | | | 2H), 1.49 (t, J = 7.2 Hz, 3H). |
| 111 | H | SCH₃ | CF₃ | H | CF₃ | N | ¹H NMR (300 MHz, Chloroform-d) δ |
| | | | | | | | 8.80 (s, 1H), 8.73 (d, J = 2.4 |
| | | | | | | | Hz, 1H), 8.65 (s, 1H), 7.82 |
| | | | | | | | (s, 1H), 7.52 (s, 1H), 6.85 (d, J = 2.4 |
| | | | | | | | Hz, 1H), 2.61 (s, 3H). |
| 112 | H | 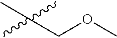 [SMILES: *COC] | CF₃ | F | CF₃ | H | ¹H NMR (300 MHz, Chloroform-d) δ 8.71 (s, 1H), 7.61 (s, 1H), 7.51-7.26 (m, 3H), 7.01 (s, 1H), 4.57 (s, 2H), 3.50 (s, 3H). |
| 113 | H | 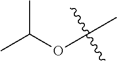 [SMILES: *OC(C)C] | CF₃ | H | CF₃ | N | ¹H NMR (300 MHz, Chloroform-d) δ 8.78 (s, 1H), 8.72 (d, J = 2.4 Hz, 1H), 8.65 (d, J = 2.4 Hz, 1H), 7.82 (d, J = 2.4 Hz, |
| | | | | | | | 1H), 7.23 (d, J = 1.8 Hz, 1H), 6.49 |
| | | | | | | | (d, J = 1.8 Hz, 1H), 4.81-4.71 |
| | | | | | | | (m, 1H), 1.43 (d, J = 6.0 Hz, 6H). |
| 114 | H | 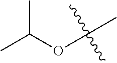 [SMILES: *OC(C)C] | CF₃ | H | CF₃ | CH | ¹H NMR (300 MHz, Chloroform-d) δ 8.72 (s, 1H), 7.59-7.42 (m, 3H), 7.39-7.32 (m, 1H), 7.18 (d, J = 1.8 Hz, 1H), 6.40 (d, J = 1.8 Hz, 1H), 4.79-4.68 (m, 1H), 1.42 (d, J = 6.0 Hz, 6H). |
| 115 | H | CH₃ | CHF₂ | H | CF₃ | CH | ¹H NMR (300 MHz, Chloroform-d) δ |
| | | | | | | | 8.76 (s, 1H), 7.56-7.49 (m, |
| | | | | | | | 2H), 7.44-7.25 (m, 3H), |
| | | | | | | | 76.89-6.47 (m, 2H), 2.48 (s, 3H). |
| 116 | H | CH₃ | F | H | CF₃ | CH | ¹H NMR (300 MHz, Chloroform-d) δ |
| | | | | | | | 8.83 (s, 1H), 7.50 (s, 1H), |
| | | | | | | | 7.42-7.36 (m, 1H), 7.02-6.88 |
| | | | | | | | (m, 3H), 6.78 (s, 1H), 2.47 (s, 3H). |
| 117 | H | CH₃ | OCHF₂ | F | CF₃ | CH | ¹H NMR (300 MHz, Chloroform-d) δ |
| | | | | | | | 8.78 (s, 1H), 7.50 (s, 1H), |
| | | | | | | | 7.22-7.13 (m, 2H), 7.05-6.98 |
| | | | | | | | (m, 1H), 6.79 (s, 1H), 6.60 (t, J = 75.0 |
| | | | | | | | Hz, 1H), 2.48 (s, 3H). |
| 118 | H | CH₃ | CH₃ | H | CF₃ | CH | ¹H NMR (300 MHz, Chloroform-d) δ |
| | | | | | | | 8.88 (s, 1H), 7.46 (s, 1H), |
| | | | | | | | 7.33 (d, J = 7.8 Hz, 1H), 7.08 |
| | | | | | | | (d, J = 7.8 Hz, 1H), 6.98-6.92 (m, |
| | | | | | | | 2H), 6.69 (s, 1H), 2.44 |
| | | | | | | | (s, 3H), 2.39 (s, 3H). |
| 119 | H | CH₃ | CF₃ | H | CHF₂ | CH | ¹H NMR (300 MHz, Chloroform-d) δ |
| | | | | | | | 8.72 (s, 1H), 7.62-7.44 (m, |
| | | | | | | | 4H), 7.40-7.33 (m, 1H), |
| | | | | | | | 6.80 (s, 1H), 6.76 (t, J = 53.4 Hz, |
| | | | | | | | 1H), 2.48 (s, 3H). |
| 120 | H | CH₃ | CF₃ | F | CF₃ | CH | ¹H NMR (300 MHz, Chloroform-d) δ |
| | | | | | | | 8.71 (s, 1H), 7.61-7.24 (m, 4H), |
| | | | | | | | 6.82 (s, 1H), 2.49 (s, 3H). |
| 121 | H | CH₃ | Cl | H | CF₃ | CH | ¹H NMR (300 MHz, Chloroform-d) δ |
| | | | | | | | 8.82 (s, 1H), 7.50 (s, 1H), |
| | | | | | | | 7.39-7.33 (m, 1H), 7.26-7.19 |
| | | | | | | | (m, 2H), 7.12-7.02 (m, 1H), 6.77 |
| | | | | | | | (s, 1H), 2.47 (s, 3H). |
| 122 | H | CH₃ | OCF₃ | H | CF₃ | CH | ¹H NMR (300 MHz, Chloroform-d) δ |
| | | | | | | | 8.77 (s, 1H), 7.53-7.41 |
| | | | | | | | (m, 2H), 7.18-7.05 (m, 3H), |
| | | | | | | | 6.81 (s, 1H), 2.48 (s, 3H). |
| 123 | H | CH₃ | OCF₃ | F | CF₃ | CH | ¹H NMR (300 MHz, Chloroform-d) δ |
| | | | | | | | 8.74 (s, 1H), 7.51 (s, 1H), |
| | | | | | | | 7.30-7.19 (m, 2H), 7.13-7.08 |
| | | | | | | | (m, 1H), 6.81 (s, 1H), 2.49 (s, 3H). |
| 124 | H | CH₃ | OCHF₂ | H | CF₃ | CH | ¹H NMR (300 MHz, Chloroform-d) δ |
| | | | | | | | 8.81 (s, 1H), 7.50 (s, 1H), 7.42 |
| | | | | | | | (t, J = 8.1 Hz, 1H), 7.04- |
| | | | | | | | 7.00 (m, 3H), 6.79 (s, 1H), 6.55 (t, |
| | | | | | | | J = 73.2 Hz, 1H), 2.48 (s, 3H). |
| 125 | CH₃ | H | CHF₂ | H | CF₂CF₃ | CH | ¹H NMR (300 MHz, Chloroform-d) δ |
| | | | | | | | 8.63 (s, 1H), 7.76 (d, J = |
| | | | | | | | 7.8 Hz, 1H), 7.57-7.50 (m, 2H), |
| | | | | | | | 7.41-7.38 (m, 2H), 7.31-7.27 |
| | | | | | | | (m, 1H), 6.68 (t, J = 56.1 |
| | | | | | | | Hz, 1H), 2.44 (s, 3H). |
| 126 | CH₃ | H | CF₃ | F | CF₂CF₃ | CH | ¹H NMR (300 MHz, Chloroform-d) δ |
| | | | | | | | 8.62 (s, 1H), 7.78 (d, J = |
| | | | | | | | 7.8 Hz, 1H), 7.58 (d, J = 7.8 |
| | | | | | | | Hz, 1H), 7.48-7.45 (m, 1H), 7.39- |
| | | | | | | | 7.34 (m, 1H), 7.31-7.25 |
| | | | | | | | (m, 1H), 2.44 (s, 3H). |
| 127 | CH₃ | H | OCHF₂ | H | CF₂CF₃ | CH | ¹H NMR (300 MHz, Chloroform-d) δ |
| | | | | | | | 8.69 (s, 1H), 7.76 (d, J = |
| | | | | | | | 7.8 Hz, 1H), 7.55 (d, J = 7.8 |
| | | | | | | | Hz, 1H), 7.46-7.26 (m, 1H), |
| | | | | | | | 7.05-7.02 (m, 3H), 6.55 (t, J = |
| | | | | | | | 73.2 Hz, 1H), 2.42 (s, 3H). |
| 128 | CH₂CH₃ | H | F | H | CHF₂ | CH | ¹H NMR (300 MHz, Chloroform-d) δ |
| | | | | | | | 8.65 (s, 1H), 7.76 (d, J = 7.8 |
| | | | | | | | Hz, 1H), 7.55 (d, J = 7.8 |
| | | | | | | | Hz, 1H), 7.42-7.34 (m, 1H), |
| | | | | | | | 7.00-6.89 (m, 3H), 6.75 (t, J = |
| | | | | | | | 53.4 Hz, 1H), 2.80 (q, J = 7.5 Hz, |
| | | | | | | | 2H), 1.33 (t, J = 7.5 Hz, 3H). |
| 129 | CH₂CH₃ | H | F | H | CF₃ | CH | ¹H NMR (300 MHz, Chloroform-d) δ |
| | | | | | | | 8.66 (s, 1H), 7.77 (d, J = 7.8 |
| | | | | | | | Hz, 1H), 7.57 (d, J = 7.8 |
| | | | | | | | Hz, 1H), 7.42-7.34 (m, 1H), |
| | | | | | | | 7.03-6.86 (m, 3H), 2.80 (q, J = 7.5 |
| | | | | | | | Hz, 2H), 1.33 (t, J = 7.5 Hz, 3H). |
| 130 | CH₂CH₃ | H | Cl | H | CF₃ | CH | ¹H NMR (300 MHz, Chloroform-d) δ |
| | | | | | | | 8.65 (s, 1H), 7.77 (d, J = 7.5 |
| | | | | | | | Hz, 1H), 7.56 (d, J = 7.5 |
| | | | | | | | Hz, 1H), 7.39-7.33 (m, 1H), |
| | | | | | | | 7.26-7.20 (m, 2H), 7.08-7.03 |
| | | | | | | | (m, 1H), 2.80 (q, J = 7.2 Hz, 2H), |
| | | | | | | | 1.32 (t, J = 7.2 Hz, 3H). |
| 131 | CH₂CH₃ | H | Cl | H | CHF₂ | CH | ¹H NMR (300 MHz, Chloroform-d) δ |
| | | | | | | | 8.65 (s, 1H), 7.76 (d, J = 7.8 |
| | | | | | | | Hz, 1H), 7.55 (d, J = 7.8 |
| | | | | | | | Hz, 1H), 7.40-7.31 (m, 1H), |
| | | | | | | | 7.25-7.05 (m, 3H), 6.75 (t, J = |
| | | | | | | | 53.4 Hz, 1H), 2.80 (q, J = 7.5 Hz, |
| | | | | | | | 2H), 1.33 (t, J = 7.5 Hz, 3H). |
| 132 | CH₂CH₃ | H | OCHF₂ | F | CF₃ | CH | ¹H NMR (300 MHz, Chloroform-d) δ |
| | | | | | | | 8.65 (s, 1H), 7.77 (d, J = 7.8 |
| | | | | | | | Hz, 1H), 7.57 (d, J = 7.8 |
| | | | | | | | Hz, 1H), 7.26-7.12 (m, 2H), |
| | | | | | | | 7.08-6.98 (m, 1H), 6.61 (t, |
| | | | | | | | J = 75.0 Hz, 1H), 2.80 (q, J = 7.5 Hz, |
| | | | | | | | 2H), 1.33 (t, J = 7.5 Hz, 3H). |
| 133 | CH₂CH₃ | H | OCHF₂ | H | CF₃ | CH | ¹H NMR (300 MHz, Chloroform-d) δ |
| | | | | | | | 8.66 (s, 1H), 7.78 (d, J = 7.8 |
| | | | | | | | Hz, 1H), 7.58 (d, J = 7.8 Hz, |
| | | | | | | | 1H), 7.47-7.37 (m, 1H), 7.11-6.96 |
| | | | | | | | (m, 3H), 6.56 (t, J = 75.0 |
| | | | | | | | Hz, 1H), 2.80 (q, J = 7.5 Hz, 2H), |
| | | | | | | | 1.34 (t, J = 7.5 Hz, 3H). |
| 134 | CH₂CH₃ | H | CH₃ | H | CF₃ | CH | ¹H NMR (300 MHz, Chloroform-d) δ |
| | | | | | | | 8.64 (s, 1H), 7.74 (d, J = |
| | | | | | | | 7.8 Hz, 1H), 7.51 (d, J = 7.8 Hz, |
| | | | | | | | 1H), 7.38-7.27 (m, 1H), 7.12- |
| | | | | | | | 7.02 (m, 1H), 7.02-6.88 (m, |
| | | | | | | | 2H), 2.80 (q, J = 7.5 Hz, 2H), 2.39 |
| | | | | | | | (s, 3H), 1.33 (t, J = 7.5 Hz, 3H). |
| 135 | CH₂CH₃ | H | CF₃ | F | CF₃ | CH | ¹H NMR (300 MHz, Chloroform-d) δ |
| | | | | | | | 8.59 (s, 1H), 7.80 (d, J = |
| | | | | | | | 7.8 Hz, 1H), 7.60 (d, J = 7.8 Hz, |
| | | | | | | | 1H), 7.52-7.41 (m, 1H), 7.41- |
| | | | | | | | 7.27 (m, 2H), 2.82 (q, J = 7.5 |
| | | | | | | | Hz, 2H), 1.35 (t, J = 7.5 Hz, 3H). |
| 136 | CH₂CH₃ | H | OCF₃ | F | CF₃ | CH | ¹H NMR (300 MHz, Chloroform-d) δ |
| | | | | | | | 8.61 (s, 1H), 7.79 (d, J = 7.8 |
| | | | | | | | Hz, 1H), 7.59 (d, J = 7.8 |
| | | | | | | | Hz, 1H), 7.30-7.27 (m, 1H), |
| | | | | | | | 7.24-7.20 (m, 1H), 7.15-7.06 (m, |
| | | | | | | | 1H), 2.83 (q, J = 7.2 Hz, 3H), 1.34 |
| | | | | | | | (t, J = 7.2 Hz, 3H). |
| 137 | CH₂CH₃ | H | CHF₂ | H | CF₃ | CH | ¹H NMR (300 MHz, Chloroform-d) δ |
| | | | | | | | 8.60 (s, 1H), 7.78 (d, J = |
| | | | | | | | 7.8 Hz, 1H), 7.58-7.49 (m, 2H), |
| | | | | | | | 7.42-7.37 (m, 2H), 7.30- |
| | | | | | | | 7.27 (m, 1H), 6.69 (t, J = 56.1 Hz, |
| | | | | | | | 1H), 2.82 (q, J = 7.5 Hz , 2H), 1.34 |
| | | | | | | | (t, J = 7.5 Hz, 3H). |
| 138 | 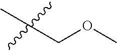 [SMILES: *COC] | H | F | H | CF₃ | CH | ¹H NMR (300 MHz, Chloroform-d) δ 8.66 (s, 1H), 8.05 (d, J = 8.1 Hz, 1H), 7.64 (d, J = 8.1 |
| | | | | | | | Hz, 1H), 7.50-7.33 (m, 1H), |
| | | | | | | | 7.08-6.88 (m, 3H), |
| | | | | | | | 4.64 (s, 2H), 3.54 (s, 3H). |
| 139 | 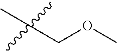 [SMILES: *COC] | H | Cl | H | CF₃ | CH | ¹H NMR (300 MHz, Chloroform-d) δ 8.66 (s, 1H), 8.04 (d, J = 7.8 Hz, 1H), 7.64 (d, J = 7.8 |
| | | | | | | | Hz, 1H), 7.41-7.38 (m, 1H), |
| | | | | | | | 7.32-7.19 (m, 2H), 7.10-7.04 |
| | | | | | | | (m, 1H), 4.64 (s, 2H), 3.54 (s, 3H). |
| 140 | 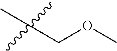 [SMILES: *COC] | H | CF₃ | H | 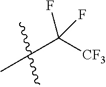 [SMILES: *C(F)(F)C(F)(F)F] | N | ¹H NMR (300 MHz, Chloroform-d) δ 8.89-8.69 (m, 2H), 8.63 (s, 1H), 8.11 (d, J = 7.8 Hz, 1H), 7.85 (s, 1H), 7.75 (d, J = 7.8 Hz, 1H), 4.68 (s, 2H), 3.55 (s, 3H). |
| 141 | 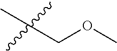 [SMILES: *COC] | H | CF₃ | H | CF₂CF₃ | CH | ¹H NMR (300 MHz, Chloroform-d) δ 8.63 (s, 1H), 8.07 (d, J = 7.8 Hz, 1H), 7.68 (d, J = 7.8 Hz, 1H), 7.57-7.36 (m, 4H), 4.67 (s, 2H), 3.55 (s, 3H). |
| 142 | 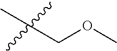 [SMILES: *COC] | H | CF₃ | H | CF₃ | N | ¹H NMR (300 MHz, Chloroform-d) δ 8.83 (s, 1H), 8.75 (d, J = 2.4 Hz, 1H), 8.60 (s, 1H), 8.11 (d, J = 8.1 Hz, 1H), 7.88-7.82 |
| | | | | | | | (m, 1H), 7.73 (d, J = 8.1 Hz, |
| | | | | | | | 1H), 4.67 (s, 2H), 3.55 (s, 3H). |
| 143 | 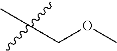 [SMILES: *COC] | H | CF₃ | F | CF₃ | CH | ¹H NMR (300 MHz, Chloroform-d) δ 8.60 (s, 1H), 8.06 (d, J = 7.8 Hz, 1H), 7.67 (d, J = 7.8 |
| | | | | | | | Hz, 1H), 7.49-7.42 (m, 1H), |
| | | | | | | | 7.42-7.34 (m, 1H), 7.33-7.27 |
| | | | | | | | (m, 1H), 4.65 (s, 2H), 3.54 (s, 3H). |
| 144 | 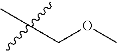 [SMILES: *COC] | H | CHF₂ | H | CF₃ | CH | ¹H NMR (300 MHz, Chloroform-d) δ 8.60 (s, 1H), 8.05 (d, J = 7.8 Hz, 1H), 7.64 (d, J = 7.8 Hz, 1H), 7.58-7.48 (m, 1H), |
| | | | | | | | 7.44-7.34 (m, 2H), 7.34-7.27 (m, |
| | | | | | | | 1H), 6.68 (t, J = 57.0 Hz, 1H), |
| | | | | | | | 4.66 (s, 2H), 3.55 (s, 3H). |
| 145 | 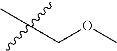 [SMILES: *COC] | H | OCF₃ | H | CF₃ | CH | ¹H NMR (300 MHz, Chloroform-d) δ 8.63 (s, 1H), 8.06 (d, J = 7.8 Hz, 1H), 7.66 (d, J = 7.8 Hz, 1H), 7.52-7.42 (m, 1H), |
| | | | | | | | 7.20-7.09 (m, 3H), |
| | | | | | | | 4.66 (s, 2H), 3.54 (s, 3H). |
| 146 | 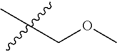 [SMILES: *COC] | H | OCF₃ | F | CF₃ | CH | ¹H NMR (300 MHz, Chloroform-d) δ 8.64 (s, 1H), 8.08 (d, J = 8.1 Hz, 1H), 7.68 (d, J = 8.1 Hz, 1H), 7.32-7.21 (m, 2H), |
| | | | | | | | 7.18-7.09 (m, 1H), |
| | | | | | | | 4.66 (s, 2H), 3.56 (s, 3H). |
| 147 | 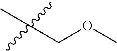 [SMILES: *COC] | H | OCHF₂ | F | CF₃ | CH | ¹H NMR (300 MHz, Chloroform-d) δ 8.66 (s, 1H), 8.04 (d, J = 8.1 Hz, 1H), 7.65 (d, J = 8.1 Hz, 1H), 7.29-7.23 (m, 1H), |
| | | | | | | | 7.24-7.15 (m, 1H), 7.06-7.00 |
| | | | | | | | (m, 1H), 6.61 (t, J = 73.2 Hz, 1H), |
| | | | | | | | 4.64 (s, 2H), 3.54 (s, 3H). |
| 148 | 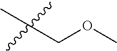 [SMILES: *COC] | H | OCHF₂ | H | CF₃ | CH | ¹H NMR (300 MHz, Chloroform-d) δ 8.66 (s, 1H), 8.08 (d, J = 7.8 Hz, 1H), 7.65 (d, J = 7.8 Hz, 1H), 7.48-7.37 (m, 1H), |
| | | | | | | | 7.10-6.99 (m, 3H), 6.55 (t, J = 73.2 |
| | | | | | | | Hz, 1H), 4.65 (s, 2H), 3.54 (s, |
| | | | | | | | 3H). |
| 149 | 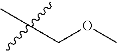 [SMILES: *COC] | H | CH₃ | H | CF₃ | CH | ¹H NMR (300 MHz, Chloroform-d) δ 8.65 (s, 1H), 8.01 (d, J = 7.8 Hz, 1H), 7.59 (d, J = 7.8 Hz, 1H), 7.34-7.21 (m, 1H), |
| | | | | | | | 7.08 (d, J = 7.5 Hz, 1H), 7.02- |
| | | | | | | | 6.92 (m, 2H), 4.65 (s, 2H), 3.54 (s, |
| | | | | | | | 3H), 2.39 (s, 3H). |

Several methods for preparing the compound of the present invention are illustrated in detail in the following schemes and examples. Raw materials can be purchased on the market or can be prepared by methods known in literatures or as shown in the detailed description. A person skilled in the art should understand that other synthetic routes may also be used to synthesize the compound of the present invention. Although the specific raw materials and conditions in the synthetic route have been illustrated below, they may be easily replaced with other similar raw materials and conditions. Various isomers and the like of the compound resulted from these modifications or variants of the preparation method of the present invention are all included in the scope of the present invention. In addition, the preparation methods as described below can be further modified according to the disclosure of the present invention using conventional chemical methods well known to a person skilled in the art. For example, protection for appropriate groups during the reaction, etc.

Examples of methods provided below are used to enhance further understanding of the preparation methods of the present invention, and the specific substances, varieties and conditions employed are determined to be further illustrations of the present invention and not to limit its reasonable scope. Reagents used in the synthesis of the compounds shown in the following tables are either commercially available or can be easily prepared by a person of ordinary skill in the art.

Examples of representative compounds are as follows. The synthesis methods of other compounds are similar and will not be described in detail here.

### 1. Synthesis of Compound 2

(1) In a 100 ml single-necked flask, Compound 2-1 (0.5 g, 2.73 mmol, 1 eq) was dissolved in 20 ml of DMF, added with cesium carbonate (1.42 g, 4.37 mmol, 1.6 eq) and Compound 2-2 (0.56 g, 4.10 mmol, 1.5 eq), displaced by N₂, added with cuprous iodide (52.03 mg, 0.27 mmol, 0.1 eq), slowly heated up to 120° C., and reacted for 12 h. The reaction was monitored until the starting materials had disappeared. The reaction solution was cooled to room temperature naturally, added with water, extracted three times with ethyl acetate, and washed three times with saturated saline solution. The sample was dried by anhydrous sodium sulfate, isolated and purified through a silica gel column. The fraction was spin-dried to obtain 0.42 g of Compound 2-3 (64% yield).

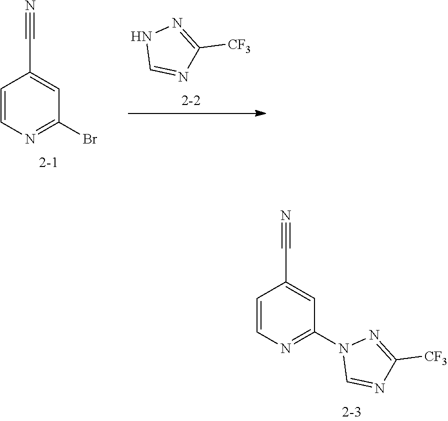 [SMILES: N#Cc1ccnc(Br)c1 ; FC(F)(F)c1nc[nH]n1 ; N#Cc1ccnc(-n2cnc(C(F)(F)F)n2)c1]

(2) Compound 2-3 (0.42 g, 1.76 mmol, 1 eq) was dissolved in 20 ml of trifluoroacetic acid, added with 30% hydrogen peroxide (0.59 g, 5.27 mmol, 3 eq), heated up to 100° C., and reacted for 12 h. The reaction was monitored until the raw materials had disappeared. The reaction solution was concentrated, and the residue was added with water, extracted three times with ethyl acetate, washed three times with saturated saline solution, and dried by anhydrous sodium sulfate. The sample was isolated and purified through a silica gel column after stirring. The fraction was spin-dried to obtain 0.31 g of Compound 2-4 (68% yield).

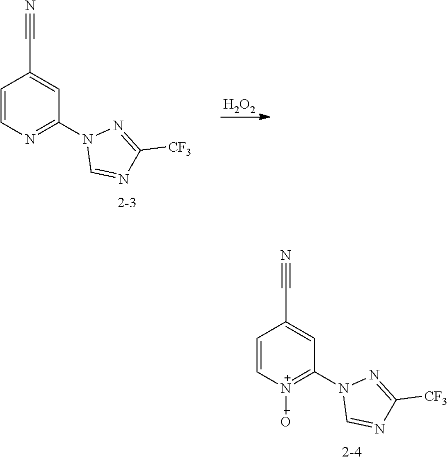 [SMILES: N#Cc1ccnc(-n2cnc(C(F)(F)F)n2)c1 ; "H2O2" ; N#Cc1cc[n+]([O-])c(-n2cnc(C(F)(F)F)n2)c1]

(3) Compound 2-4 (0.31 g, 1.21 mmol, 1 eq) was dissolved in 10 ml of phosphorus oxychloride, heated up to 80° C., and reacted for 12 h. The reaction was monitored until the raw materials had disappeared. The reaction solution was concentrated, and the residue was added with water, extracted three times with ethyl acetate, washed three times with saturated saline solution, and dried by anhydrous sodium sulfate. The sample was purified through a silica gel column after stirring. The fraction was spin-dried to obtain 0.2 g of Compound 2-5 (60% yield).

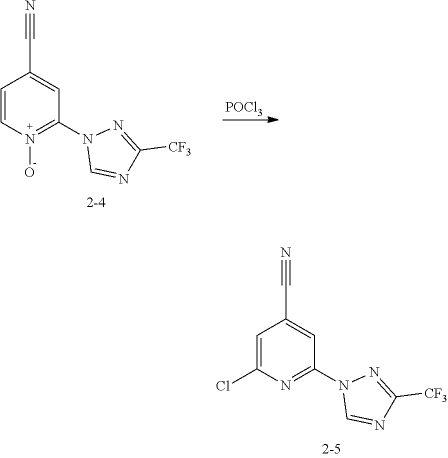

(4) Compound 2-5 (0.1 g, 0.36 mmol, 1 eq) was dissolved in 5 ml of DMF, added with potassium carbonate (0.1 g, 0.72 mmol, 2 eq), added with m-trifluoromethylphenol (0.065 g, 0.40 mmol, 1.1 eq), heated up to 120° C., and reacted for 12 h. The reaction was monitored until the raw materials had disappeared. The reaction solution was cooled to room temperature naturally, added with water, extracted three times with ethyl acetate, washed three times with saturated saline solution, and dried by anhydrous sodium sulfate. The sample was isolated and purified through a silica gel column after stirring. The fraction was spin-dried to obtain 0.053 g of Compound 2 (36% yield).

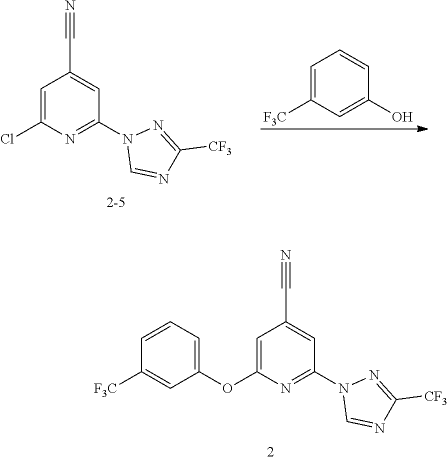 [SMILES: N#Cc1cc(Cl)nc(-n2cnc(C(F)(F)F)n2)c1 ; Oc1cccc(C(F)(F)F)c1 ; N#Cc1cc(Oc2cccc(C(F)(F)F)c2)nc(-n2cnc(C(F)(F)F)n2)c1]

### 2. Synthesis of Compound 3

(1) In a 100 ml single-necked flask, Compound 3-1 (0.5 g, 2.66 mmol, 1 eq) was dissolved in 20 ml of DMF, added with cesium carbonate (1.39 g, 4.25 mmol, 1.6 eq) and Compound 2-2 (0.55 g, 3.99 mmol, 1.5 eq), displaced by N₂, added with cuprous iodide (50.65 mg, 0.27 mmol, 0.1 eq), slowly heated up to 120° C., and reacted for 12 h. The reaction was monitored until the starting materials had disappeared. The reaction solution was cooled to room temperature naturally, added with water, extracted three times with ethyl acetate, and washed three times with saturated saline solution. The sample was dried by anhydrous sodium sulfate, isolated and purified through a silica gel column. The fraction was spin-dried to obtain 0.31 g of Compound 3-2 (47% yield).

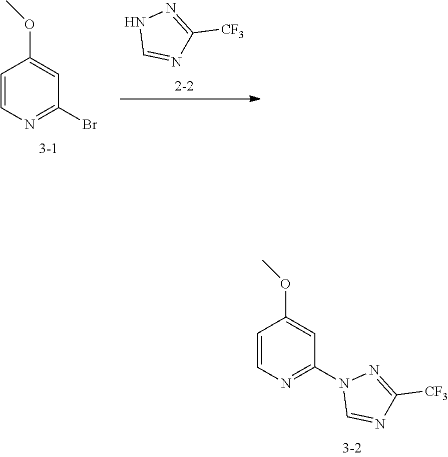

(2) Compound 3-2 (0.31 g, 1.27 mmol, 1 eq) was dissolved in 20 ml of trifluoroacetic acid, added with 30% hydrogen peroxide (0.43 g, 3.81 mmol, 3 eq), heated up to 100° C., and reacted for 12 h. The reaction was monitored until the raw materials had disappeared. The reaction solution was concentrated, and the residue was added with water, extracted three times with ethyl acetate, washed three times with saturated saline solution, and dried by anhydrous sodium sulfate. The sample was isolated and purified through a silica gel column after stirring. The fraction was spin-dried to obtain 0.2 g of Compound 3-3 (60% yield).

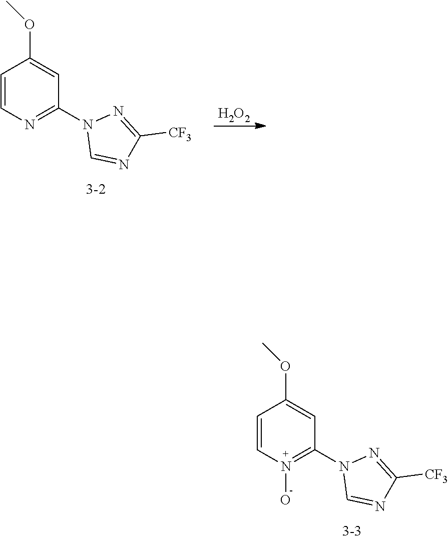

(3) Compound 3-3 (0.2 g, 0.77 mmol, 1 eq) was dissolved in 10 ml of phosphorus oxychloride, heated up to 80° C., and reacted for 12 h. The reaction was monitored until the raw materials had disappeared. The reaction solution was concentrated, and the residue was added with water, extracted three times with ethyl acetate, washed three times with saturated saline solution, and dried by anhydrous sodium sulfate. The sample was purified through a silica gel column after stirring. The fraction was spin-dried to obtain 0.15 g of Compound 3-4 (71% yield).

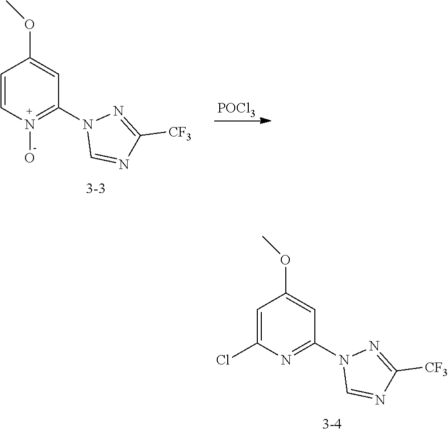

(4) Compound 3-4 (0.1 g, 0.36 mmol, 1 eq) was dissolved in 5 ml of DMF, added with potassium carbonate (0.1 g, 0.72 mmol, 2 eq), added with m-trifluoromethylphenol (0.064 g, 0.39 mmol, 1.1 eq), heated up to 12° C., and reacted for 12 h. The reaction was monitored until the raw materials had disappeared. The reaction solution was cooled to room temperature naturally, added with water, extracted three times with ethyl acetate, washed three times with saturated saline solution, and dried by anhydrous sodium sulfate. The sample was purified through a silica gel column after stirring. The fraction was spin-dried to obtain 0.045 g of Compound 3 (31% yield).

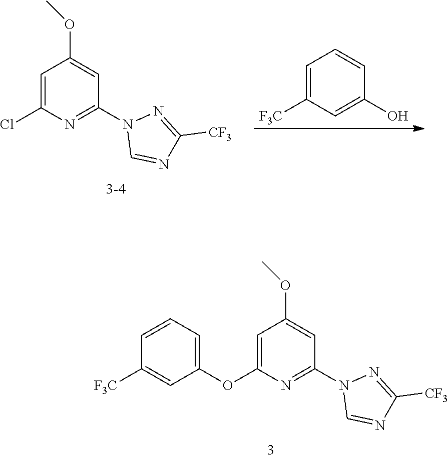 [SMILES: COc1cc(Cl)nc(-n2cnc(C(F)(F)F)n2)c1 ; Oc1cccc(C(F)(F)F)c1 ; COc1cc(Oc2cccc(C(F)(F)F)c2)nc(-n2cnc(C(F)(F)F)n2)c1]

### 3. Synthesis of Compound 8

(1) In a 50 ml single-necked flask, Compound 8-1 (1.11 g, 10 mmol, 1 eq) was dissolved in 20 ml of DMF, added with potassium carbonate (2.76 g, 20 mmol, 2 eq) and Compound 2-2 (1.38 g, 10 mmol, 1 eq), slowly heated up to 100° C., and reacted for 12 h. The reaction was monitored until the starting materials had disappeared. The reaction solution was cooled to room temperature naturally, added with water, extracted three times with ethyl acetate, and washed three times with saturated saline solution. The sample was dried by anhydrous sodium sulfate and purified through a silica gel column. The fraction was spin-dried to obtain 1.869 g of Compound 8-2 (82.1% yield).

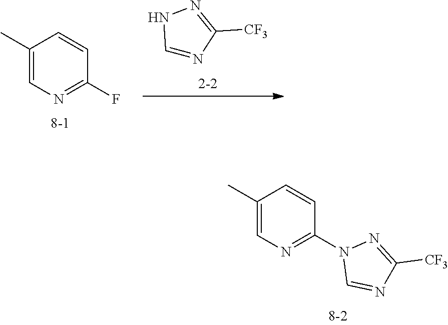

(2) Compound 8-2 (0.6 g, 2.6 mmol, 1 eq) was dissolved in 10 ml of trifluoroacetic acid, added with 30% hydrogen peroxide (0.9 g, 7.8 mmol, 3 eq), heated up to 100° C., and reacted for 12 h. The reaction was monitored until the raw materials had disappeared. The reaction solution was concentrated, and the residue was added with water, extracted three times with ethyl acetate, washed three times with saturated saline solution, and dried by anhydrous sodium sulfate. The sample was isolated and purified through a silica gel column after stirring. The fraction was spin-dried to obtain 0.45 g of Compound 8-3 (70.1% yield).

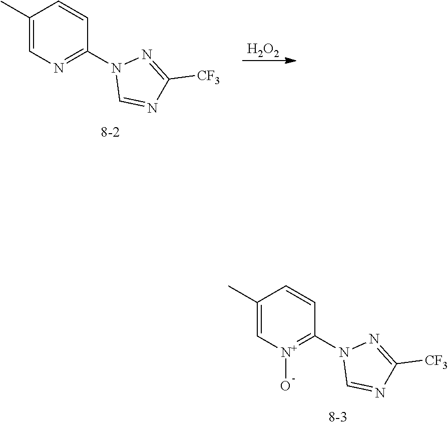

(3) Compound 8-3 (0.45 g, 1.84 mmol, 1 eq) was dissolved in 10 ml of phosphorus oxychloride, heated up to 100° C., and reacted for 12 h. The reaction was monitored until the raw materials had disappeared. The reaction solution was concentrated, and the residue was added with water, extracted three times with ethyl acetate, washed three times with saturated saline solution, and dried by anhydrous sodium sulfate. The sample was purified through a silica gel column after stirring. The fraction was spin-dried to obtain 0.32 g of Compound 8-4 (67% yield).

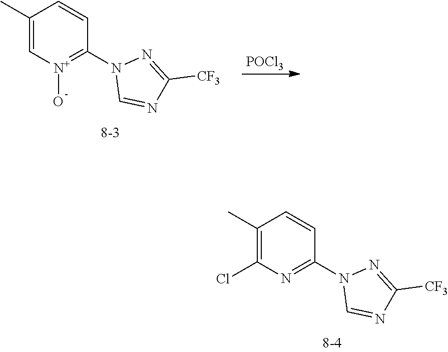 [SMILES: Cc1ccc(-n2cnc(C(F)(F)F)n2)[n+](O)c1 ; "POCl3" ; Cc1ccc(-n2cnc(C(F)(F)F)n2)nc1Cl]

(4) Compound 8-4 (0.2 g, 0.76 mmol, 1 eq) was dissolved in 10 ml of DMF, added with potassium carbonate (0.21 g, 1.52 mmol, 2 eq), added with m-trifluoromethylphenol (0.13 g, 0.76 mmol, 1 eq), heated up to 100° C., and reacted for 12 h. The reaction was monitored until the raw materials had disappeared. The reaction solution was cooled to room temperature naturally, added with water, extracted three times with ethyl acetate, washed three times with saturated saline solution, and dried by anhydrous sodium sulfate. The sample was isolated and purified through a silica gel column after stirring. The fraction was spin-dried to obtain 0.14 g of Compound 8 (47.5% yield).

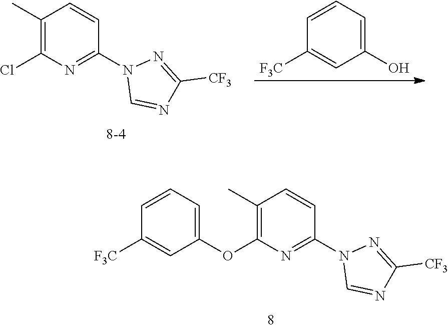 [SMILES: Cc1ccc(-n2cnc(C(F)(F)F)n2)nc1Cl ; Oc1cccc(C(F)(F)F)c1 ; Cc1ccc(-n2cnc(C(F)(F)F)n2)nc1Oc1cccc(C(F)(F)F)c1]

### 4. Synthesis of Compound 15

(1) Compound 15-1 (1.0 g, 5.35 mmol, 1 eq), 2-bromo-5-methylpyridine (0.46 g, 2.67 mmol, 0.5 eq) and cesium carbonate (3.23 g, 22.54 mmol, 2 eq) were added into a 100 ml flask, added with 20 ml of DMF for dissolution, displaced with N₂, added with cuprous iodide (101.81 mg, 0.53 mmol, 0.1 eq), heated up to 120° C., and stirred for 12 h. The reaction was monitored until completion. The reaction solution was added with water, extracted three times with ethyl acetate, washed three times with saturated saline solution, and dried by anhydrous sodium sulfate. The sample was isolated and purified through a silica gel column after stirring. The fraction was spin-dried to obtain 0.4 g of Compound 15-2 (27% yield).

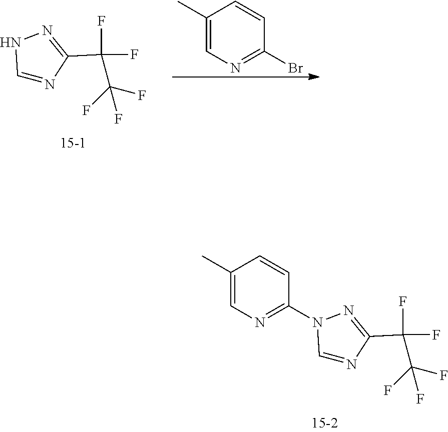

(2) Compound 15-2 (0.4 g, 1.44 mmol, 1 eq) was dissolved in 20 ml of trifluoroacetic acid, added with 30% hydrogen peroxide (0.49 g, 4.31 mmol, 3 eq), heated up to 100° C., and reacted for 12 h. The reaction was monitored until the raw materials had disappeared. The reaction solution was concentrated, and the residue was added with water, extracted three times with ethyl acetate, washed three times with saturated saline solution, and dried by anhydrous sodium sulfate. The sample was isolated and purified through a silica gel column after stirring. The fraction was spin-dried to obtain 0.33 g of Compound 15-3 (78% yield).

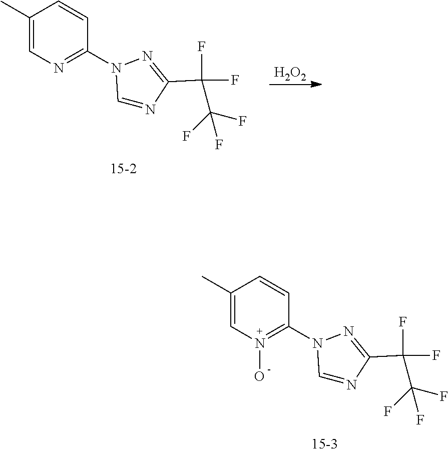

(3) Compound 15-3 (0.1 g, 0.34 mmol, 1 eq) was dissolved in 10 ml of phosphorus oxychloride, heated up to 80° C., and reacted for 12 h. The reaction was monitored until the raw materials had disappeared. The reaction solution was concentrated, and the residue was added with water, extracted three times with ethyl acetate, washed three times with saturated saline solution, and dried by anhydrous sodium sulfate. The sample was isolated and purified through a silica gel column after stirring. The fraction was spin-dried to obtain 0.08 g of Compound 15-4 (75% yield).

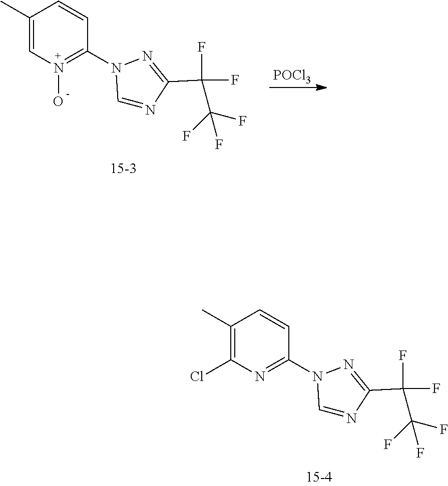

(4) Compound 15-4 (0.08 g, 0.26 mmol, 1 eq) was dissolved in 5 ml of DMF, added with potassium carbonate (0.07 g, 0.51 mmol, 2 eq), added with Compound 15-5 (0.05 g, 0.28 mmol, 1.1 eq), heated up to 120° C., and reacted for 12 h. The reaction was monitored until the raw materials had disappeared. The reaction solution was cooled to room temperature naturally, added with water, extracted three times with ethyl acetate, washed three times with saturated saline solution, and dried by anhydrous sodium sulfate. The sample was purified through a silica gel column after stirring. The fraction was spin-dried to obtain 0.051 g of Compound 15 (45% yield).

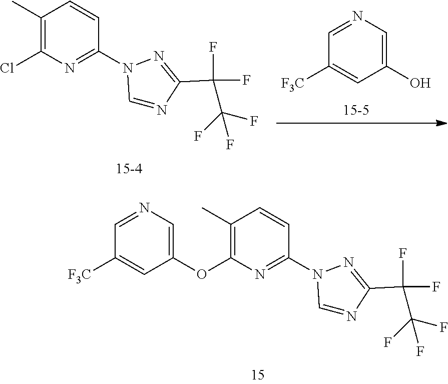 [SMILES: Cc1ccc(-n2cnc(C(F)(F)C(F)(F)F)n2)nc1Cl ; Oc1cncc(C(F)(F)F)c1 ; Cc1ccc(-n2cnc(C(F)(F)C(F)(F)F)n2)nc1Oc1cncc(C(F)(F)F)c1]

### 5. Synthesis of Compound 16

(1) In a 250 ml single-necked flask, Compound 16-1 (3.0 g, 13.45 mmol, 1 eq) was dissolved in 50 ml of DMF, added with potassium carbonate (3.72 g, 26.91 mmol, 2 eq) and Compound 2-2 (1.84 g, 13.45 mmol, 1 eq), slowly heated up to 100° C., and reacted for 12 h. The reaction was monitored until the starting materials had disappeared. The reaction solution was cooled to room temperature naturally, added with water, extracted three times with ethyl acetate, and washed three times with saturated saline solution. The sample was dried by anhydrous sodium sulfate and purified through a silica gel column. The fraction was spin-dried to obtain 3.8 g of Compound 16-2 (83% yield).

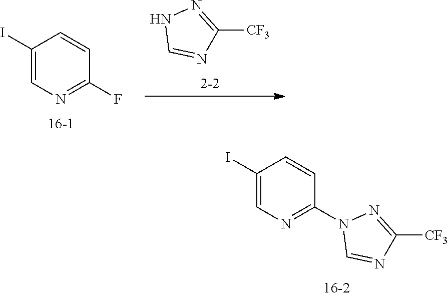 [SMILES: Fc1ccc(I)cn1 ; FC(F)(F)c1nc[nH]n1 ; FC(F)(F)c1ncn(-c2ccc(I)cn2)n1]

(2) Compound 16-2 (1 g, 2.94 mmol, 1 eq) was dissolved in 20 ml of 1,4-dioxane and 2 ml of water, added with potassium vinyltrifluoroborate (0.39 g, 2.94 mmol, 1 eq), added with cesium fluoride (0.89 g, 5.88 mmol, 2 eq), displaced with N₂, added with 1,1′-bis(diphenylphosphino) ferrocene dichloride palladium (II) dichloromethane complex (21 mg, 0.029 mmol, 0.01 eq), displaced with N₂ for three times, heated up to 100° C., and reacted for 12 h. The reaction was monitored until the starting materials had disappeared. After being concentrated, the reaction solution was added with water, extracted three times with ethyl acetate, and washed three times with saturated saline solution. The sample was dried by anhydrous sodium sulfate and purified through a silica gel column. The fraction was spin-dried to obtain 0.6 g of Compound 16-3 (86% yield).

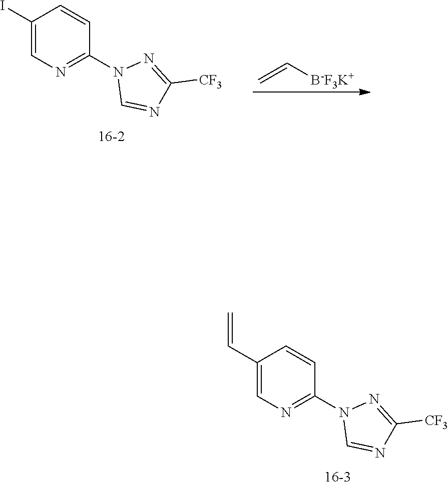

(3) Compound 16-3 (0.6 g, 2.5 mmol, 1 eq) was dissolved in 20 ml of methanol, added with palladium on carbon (catalytic amount), displaced with H₂ for three times, and stirred for 12 h at room temperature. The reaction was monitored until the raw materials had disappeared. The reaction solution was filtered by diatomaceous earth and concentrated to obtain 0.57 g of the crude product 16-4.

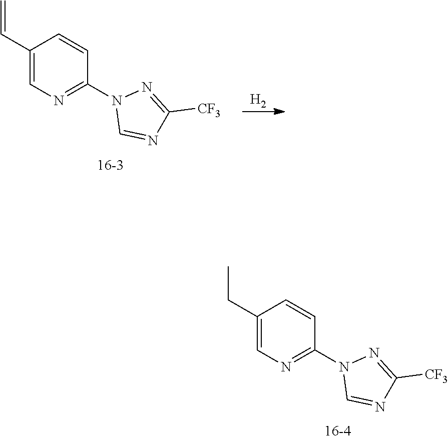 [SMILES: C=Cc1ccc(-n2cnc(C(F)(F)F)n2)nc1 ; "H2" ; CCc1ccc(-n2cnc(C(F)(F)F)n2)nc1]

(4) Compound 16-4 (0.57 g, 2.35 mmol, 1 eq) was dissolved in 10 ml of trifluoroacetic acid, added with 30% hydrogen peroxide (0.8 g, 7.06 mmol, 3 eq), heated up to 100° C., and reacted for 12 h. The reaction was monitored until the raw materials had disappeared. The reaction solution was concentrated, and the residue was added with water, extracted three times with ethyl acetate, washed three times with saturated saline solution, and dried by anhydrous sodium sulfate. The sample was purified through a silica gel column after stirring. The fraction was spin-dried to obtain 0.45 g of Compound 16-5 (74% yield).

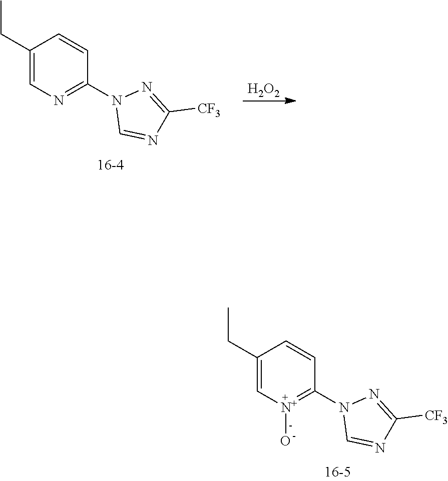

(5) Compound 16-5 (0.45 g, 1.74 mmol, 1 eq) was dissolved in 10 ml of phosphorus oxychloride, heated up to 100° C., and reacted for 12 h. The reaction was monitored until the raw materials had disappeared. The reaction solution was concentrated, and the residue was added with water, extracted three times with ethyl acetate, washed three times with saturated saline solution, and dried by anhydrous sodium sulfate. The sample was purified through a silica gel column after stirring. The fraction was spin-dried to obtain 0.32 g of Compound 16-6 (66% yield).

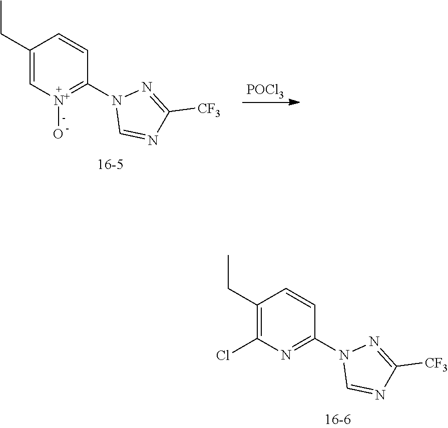 [SMILES: CCc1ccc(-n2cnc(C(F)(F)F)n2)[n+](O)c1 ; "POCl3" ; CCc1ccc(-n2cnc(C(F)(F)F)n2)nc1Cl]

(6) Compound 16-6 (0.1 g, 0.36 mmol, 1 eq) was dissolved in 5 ml of DMF, added with potassium carbonate (0.1 g, 0.72 mmol, 2 eq), added with m-trifluoromethylphenol (0.06 g, 0.36 mmol, 1 eq), heated up to 100° C., and reacted for 12 h. The reaction was monitored until the raw materials had disappeared. The reaction solution was cooled to room temperature naturally, added with water, extracted three times with ethyl acetate, washed three times with saturated saline solution, and dried by anhydrous sodium sulfate. The sample was purified through a silica gel column after stirring. The fraction was spin-dried to obtain 0.062 g of Compound 16 (42% yield).

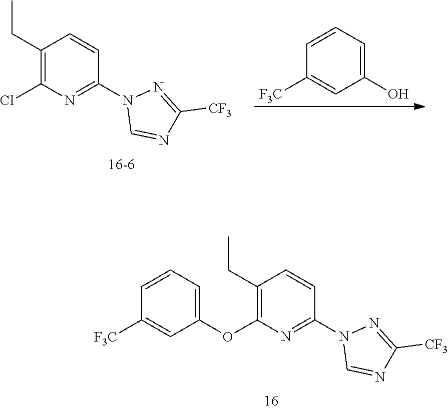 [SMILES: CCc1ccc(-n2cnc(C(F)(F)F)n2)nc1Cl ; Oc1cccc(C(F)(F)F)c1 ; CCc1ccc(-n2cnc(C(F)(F)F)n2)nc1Oc1cccc(C(F)(F)F)c1]

### 6. Synthesis of Compound 18

(1) Compound 18-1 (1.0 g, 7.51 mmol), 2-fluoro-5-bromopyridine (1.32 g, 7.51 mmol) and potassium carbonate (3.23 g, 22.54 mmol) were added into a 100 ml flask, added with 20 ml of DMF for dissolution, and stirred at 120° C. for 10 h. After the In-Process Control was completed, the reaction solution was washed with water, and then added with EA for extraction (extracted 3 times with suitable amount of EA). Next, the EA phase was washed twice with saturated saline solution and spin-dried to obtain the white solid 18-2 (800 mg, 2.77 mmol).

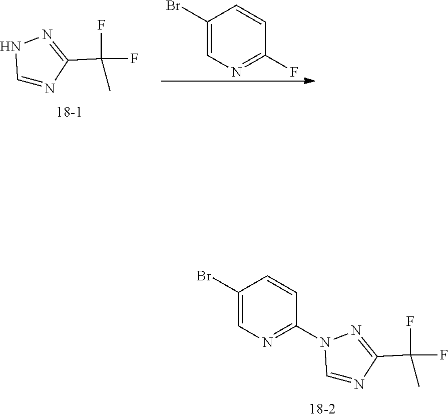

(2) Compound 18-2 (800 mg, 2.77 mmol), potassium vinyltrifluoroborate (0.44 g, 3.32 mmol) and potassium carbonate (1.19 g, 8.3 mmol) were added into a clean flask, added with 20 ml of 1,4-dioxane and 2 ml of water, and then protected by nitrogen and evacuated for 2-3 times. After evacuation, the reaction solution was added with Pd(dppf)Cl₂ dichloromethane complex (113.0 mg, 138.37 μmol), evacuated again, heated up to 85° C., and stayed overnight. After the In-Process Control was completed, the solution was treated by decompressing distillation to dry the solvent, washed with water, and extracted 3 times with EA. Then the EA phase was washed twice with saturated saline solution, added with anhydrous sodium sulfate for drying, added with silica gel and stirred, and purified by normal phase chromatography to obtain Compound 18-3 (530 mg, 2.24 mmol).

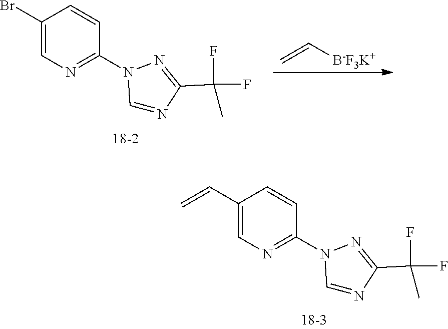

(3) Compound 18-3 (530 mg, 2.24 mmol) was added into 20 ml of methanol for dissolution, then added with 10% palladium on carbon (100 mg), displaced three times by a nitrogen balloon, and stirred overnight at room temperature. After the In-Process Control was completed, diatomaceous earth was used to filter out palladium on carbon, and the solution was spin-dried to obtain the pale yellow solid 18-4 (500 mg, 2.10 mmol).

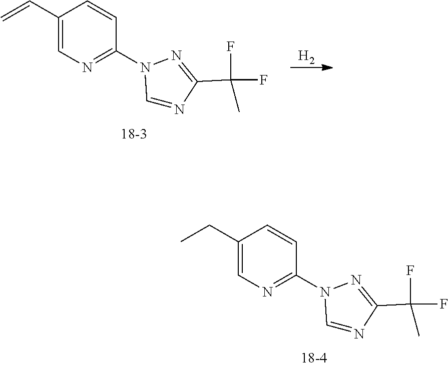 [SMILES: C=Cc1ccc(-n2cnc(C(C)(F)F)n2)nc1 ; "H2" ; CCc1ccc(-n2cnc(C(C)(F)F)n2)nc1]

(4) Compound 18-4 (500 mg, 2.10 mmol) was added into a clean flask, added with 8 ml of trifluoroacetic acid for dissolution, added dropwise with hydrogen peroxide (713.85 mg, 20.99 mmol) at room temperature, and stirred at 100° C. for 10 h. After the In-Process Control was completed, the solution was washed with water and then added with EA for extraction (extracted 3 times with suitable amount of EA). Next, the EA phase was washed twice with suitable amount of saturated saline solution and purified by normal phase chromatography to obtain the white solid 18-5 (380 mg, 1.49 mmol).

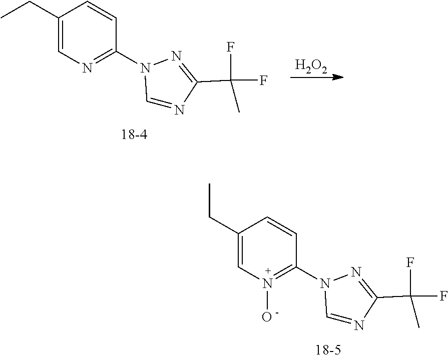

(5) Compound 18-5 (380 mg, 1.49 mmol) was added into a 50 ml flask, added with 8 ml of POCl₃ for dissolution, and stirred at 80° C. for 10 h. After the In-Process Control was completed, water was slowly added to quench the reaction and then EA was added for extraction (extracted 3 times with suitable amount of EA). Next, the EA phase was washed twice with suitable amount of saturated saline solution and spin-dried to obtain the white solid Compound 18-6 (120.0 mg, 440.7 umol).

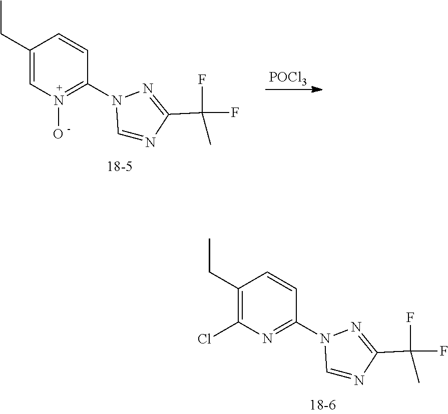

(6) Compound 18-6 (120.0 mg, 440.7 μmol), m-trifluoromethylphenol (85.61 mg, 528.1 μmol) and potassium carbonate (189.11 mg, 1.32 mmol) were added into a clean flask, added with 8 ml of DMF for dissolution, and stirred at 100° C. for 10 h. After the In-Process Control was completed, the solution was washed with water and then extracted 3 times with EA. Next, the EA phase was washed twice with saturated saline solution and purified through a silica gel column after concentration to obtain the white solid Compound 18 (50.0 mg, 125.5 μmol).

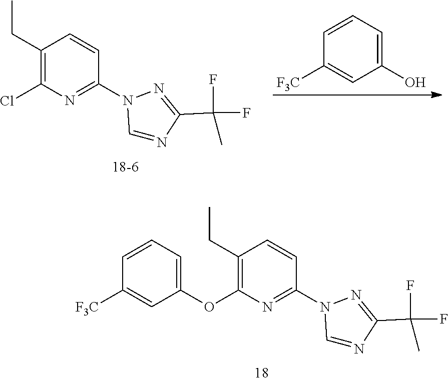 [SMILES: CCc1ccc(-n2cnc(C(C)(F)F)n2)nc1Cl ; Oc1cccc(C(F)(F)F)c1 ; CCc1ccc(-n2cnc(C(C)(F)F)n2)nc1Oc1cccc(C(F)(F)F)c1]

### 7. Synthesis of Compound 20

Compound 16-6 (150 mg, 0.543 mmol) was dissolved in 5 ml of DMF, sequentially added with Compound 20-1 (170 mg, 1.2 eq, 0.651 mmol), potassium carbonate (225 mg, 3.0 eq, 1.63 mmol), stirred and reacted at 120° C. overnight. After the In-Process Control was completed, water and EA were added into the system for extraction. The organic phases were combined and washed three times with half-saturated saline solution. The organic phase was dried, concentrated, and purified by normal phase chromatography to obtain Compound 20 (67 mg, 0.133 mmol).

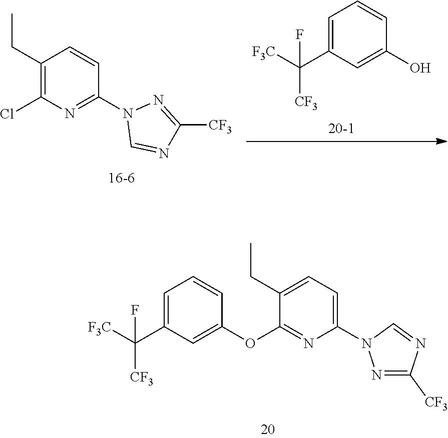 [SMILES: CCc1ccc(-n2cnc(C(F)(F)F)n2)nc1Cl ; Oc1cccc(C(F)(C(F)(F)F)C(F)(F)F)c1 ; CCc1ccc(-n2cnc(C(F)(F)F)n2)nc1Oc1cccc(C(F)(C(F)(F)F)C(F)(F)F)c1]

### 8. Synthesis of Compound 22

(1) Compound 16-2 (5.3 g, 15.59 mmol, 1 eq) was dissolved in 20 ml of toluene and 1 ml of water, added with cyclopropylboric acid (1.61 g, 18.7 mmol, 1.2 eq), added with potassium phosphate (11.58 g, 54.55 mmol, 3.5 eq), displaced with N₂, added with palladium acetate (129.73 mg, 0.779 mmol, 0.05 eq) and tricyclohexylphosphine (437.09 mg, 1.56 mmol, 0.1 eq), displaced with N₂ for 3 times, heated up to 80° C. and reacted for 12 h. The reaction was monitored until the starting materials had disappeared. After being spin-dried, the solution was added with water, extracted three times with ethyl acetate, and washed three times with saturated saline solution. The sample was dried by anhydrous sodium sulfate and then purified through a silica gel column. The fraction was spin-dried to obtain 2.5 g of Compound 22-1 (64% yield).

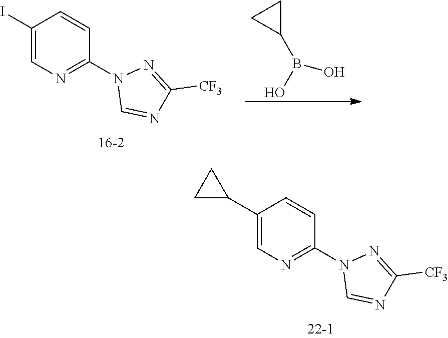 [SMILES: FC(F)(F)c1ncn(-c2ccc(I)cn2)n1 ; OB(O)C1CC1 ; FC(F)(F)c1ncn(-c2ccc(C3CC3)cn2)n1]

(2) Compound 22-1 (1 g, 3.93 mmol, 1 eq) was dissolved in 20 ml of trifluoroacetic acid, added with 30% hydrogen peroxide (1.34 g, 11.8 mmol, 3 eq), heated up to 100° C., and reacted for 12 h. The reaction was monitored until the starting materials had disappeared. The reaction solution was concentrated, and the residue was added with water, extracted three times with ethyl acetate, washed three times with saturated saline solution, and dried by anhydrous sodium sulfate. The sample was then purified through a silica gel column after stirring. The fraction was spin-dried to obtain 0.6 g of Compound 22-2 (56% yield).

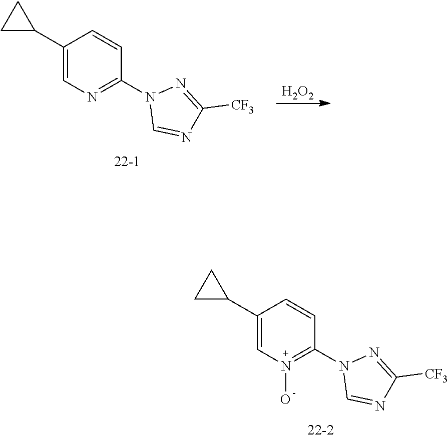

(3) Compound 22-2 (0.6 g, 2.22 mmol, 1 eq) was dissolved in 10 ml of 1,2-dichloroethane, added with phosphorus oxychloride (1.02 g, 6.66 mmol, 3 eq), heated up to 60° C., and reacted for 12 h. The reaction was monitored until the starting materials had disappeared. The reaction solution was concentrated, and the residue was added with water, extracted three times with ethyl acetate, washed three times with saturated saline solution, and dried by anhydrous sodium sulfate. The sample was then purified through a silica gel column after stirring. The fraction was spin-dried to obtain 0.5 g of Compound 22-3 (78% yield).

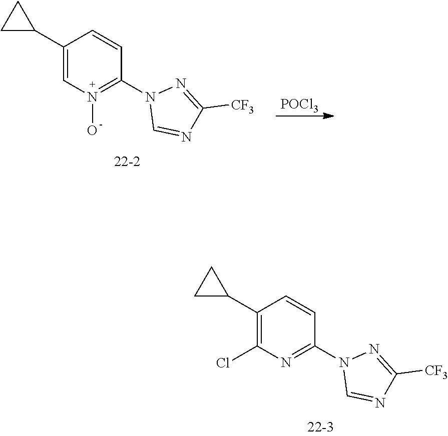

(4) Compound 22-3 (0.1 g, 0.34 mmol, 1 eq) was dissolved in 5 ml of DMF, added with potassium carbonate (0.095 g, 0.72 mmol, 2 eq), added with m-trifluoromethylphenol (0.062 g, 0.38 mmol, 1.1 eq), heated up to 120° C., and reacted for 12 h. The reaction was monitored until the raw materials had disappeared. The reaction solution was cooled to room temperature naturally, added with water, extracted three times with ethyl acetate, washed three times with saturated saline solution, and dried by anhydrous sodium sulfate. The sample was then purified through a silica gel column after stirring. The fraction was spin-dried to obtain 0.03 g of Compound 22 (21% yield).

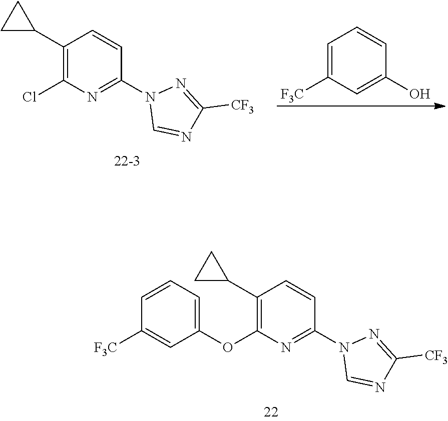 [SMILES: FC(F)(F)c1ncn(-c2ccc(C3CC3)c(Cl)n2)n1 ; Oc1cccc(C(F)(F)F)c1 ; FC(F)(F)c1cccc(Oc2nc(-n3cnc(C(F)(F)F)n3)ccc2C2CC2)c1]

### Evaluation of Biological Activity:

### (1) Post-Emergence Test Experiment:

The activity level criteria for plant damage (i.e., growth control rate) are as follows:
Level 9: completely dead;
Level 8: growth control rate is greater than or equal to 90% and less than 100%;
Level 7: growth control rate is greater than or equal to 80% and less than 90%;
Level 6: growth control rate is greater than or equal to 70% and less than 80%;
Level 5: growth control rate is greater than or equal to 50% and less than 70%;
Level 4: growth control rate is greater than or equal to 30% and less than 50%;
Level 3: growth control rate is greater than or equal to 20% and less than 30%;
Level 2: growth control rate is greater than or equal to 10% and less than 20%;
Level 1: growth control rate is less than 10%;
Level 0: no effect.

The above growth control rates are fresh weight control rates.

Monocotyledonous and dicotyledonous weed seeds (Descurainia sophia, Capsella bursa-pastoris, Abutilon theophrasti, Galium spurium, Stellaria media, Lithospermum arvense, Rorippa indica, Alopecurus aequalis, Alopecurus japonicus, Eleusine indica, Beckmannia syzigachne, Sclerochloa dura, Erigeron canadensis, Phleum paniculatum, Veronica, Bromus japonicus, Aegilops tauschii, Phalaris arundinacea, Amaranthus retroflexus, Chenopodium, Commelina communis, Cichorium endivia, Convolvulus arvensis, Cirsium arvense, Solanum nigrum, Acalypha australis, Digitaria sanguinalis, Echinochloa crusgalli, Setaria viridis, Leptochloa chinensis, Monochoria vaginalis, Sagittaria trifolia, Schoenoplectiella juncoides, Cyperus rotundus, Cyperus iria, Cyperus difformis, Fimbristylis, Portulaca oleracea, Xanthium sibiricum, Ipomoea nil, Eschenbachia japonica, etc.) and major crop seeds (wheat, corn, rice, soybean, cotton, oilseed rape, millet, sorghum, potato, sesame, castor plant, etc.) were placed in plastic pots filled with soil, and then covered with 0.5-2 cm of soil to allow them to grow in a favorable greenhouse environment. Two weeks after sowing, the test plants were treated at the stage of 2-4 leaves. The test compounds of the present invention were individually dissolved with acetone, then added with Tween-80, with 1.5 L/ha of methyl oleate emulsifiable concentrate as a synergist, diluted into solution of a certain concentration using a certain amount of water, and sprayed to plants using a spray tower. After drug application, cultivation was conducted for 3 weeks in a greenhouse and then the experimental results were collected. The compounds were applied at 250 g a.i./ha in triplicate and the results were averaged. It has been found by experimentations that some of the compounds have outstanding effects on broad-leaf and gramineous weeds at this dosage.

### (2) Pre-Emergence Test Experiment:

Monocotyledonous and dicotyledonous weed seeds (Descurainia sophia, Capsella bursa-pastoris, Abutilon theophrasti, Galium spurium, Stellaria media, Lithospermum arvense, Rorippa indica, Alopecurus aequalis, Alopecurus japonicus, Eleusine indica, Beckmannia syzigachne, Sclerochloa dura, Erigeron canadensis, Phleum paniculatum, Veronica, Bromus japonicus, Aegilops tauschii, Phalaris arundinacea, Amaranthus retroflexus, Chenopodium, Commelina communis, Cichorium endivia, Convolvulus arvensis, Cirsium arvense, Solanum nigrum, Acalypha australis, Digitaria sanguinalis, Echinochloa crusgalli, Setaria viridis, Leptochloa chinensis, Monochoria vaginalis, Sagittaria trifolia, Schoenoplectiella juncoides, Cyperus rotundus, Cyperus iria, Cyperus difformis, Fimbristylis, Portulaca oleracea, Xanthium sibiricum, Ipomoea nil, Eschenbachia japonica, etc.) and major crop seeds (wheat, corn, rice, soybean, cotton, oilseed rape, millet, sorghum, potato, sesame, castor plant, etc.) were placed in plastic pots filled with soil, and then covered with 0.5-2 cm of soil. The test compounds of the present invention were individually dissolved with acetone, then added with Tween-80, diluted into solution of a certain concentration using a certain amount of water and sprayed to plants using a spray tower. After drug application, cultivation was conducted for 3 weeks in a greenhouse and then the experimental results were collected. The compounds were applied at 60, 30, 15, and 7.5 g a.i./ha in triplicate and the results were averaged. The effects were evaluated according to the above activity level criteria. Representative data were listed in Table 2.

TABLE 2
Results of pre-emergence test experiment

| | Descurainia | | Solanum | Digitaria | Setaria | Amaranthus | | | | Dosage |
|---|---|---|---|---|---|---|---|---|---|---|
| No. | sophia | Veronica | nigrum | sanguinalis | viridis | retroflexus | Corn | Soybean | Cotton | (g a.i./ha) |
| 2 | 9 | 9 | 9 | 8 | 8 | 9 | 0 | 0 | 0 | 30 |
| 3 | 9 | 9 | 9 | 8 | 9 | 9 | 0 | 0 | 0 | 30 |
| 5 | 9 | 9 | 9 | 9 | 9 | 9 | 0 | 0 | 0 | 30 |
| 6 | 9 | 9 | 9 | 9 | 9 | 9 | 0 | 0 | 0 | 30 |
| 7 | 9 | 9 | N | 8 | 9 | 9 | 0 | 0 | 0 | 30 |
| 8 | 9 | 9 | 9 | 9 | 9 | 9 | 0 | 0 | 0 | 15 |
| 9 | 9 | 9 | 9 | 9 | 9 | 9 | 0 | 0 | 0 | 30 |
| 10 | 9 | 9 | 9 | 9 | 9 | 9 | 0 | 0 | 0 | 30 |
| 11 | 9 | 9 | 9 | 9 | 9 | 9 | 0 | 0 | 0 | 30 |
| 12 | 9 | 9 | 9 | 9 | 9 | 9 | 0 | 0 | 0 | 30 |
| 13 | 9 | 9 | N | 8 | 7 | 9 | 0 | 0 | 0 | 30 |
| 14 | 9 | 9 | 9 | 8 | 9 | 9 | 0 | 0 | 0 | 30 |
| 15 | 9 | 9 | 8 | 8 | 9 | 9 | 0 | 0 | 0 | 30 |
| 16 | 9 | 9 | 9 | 9 | 9 | 9 | 0 | 0 | 0 | 15 |
| 17 | 9 | 9 | 9 | 9 | 9 | 9 | 0 | 0 | 0 | 30 |
| 18 | 9 | 9 | 8 | 8 | 8 | 9 | 0 | 0 | 0 | 30 |
| 19 | 9 | 9 | 8 | 8 | 8 | 9 | 0 | 0 | 0 | 30 |
| 21 | 9 | 9 | 7 | 8 | 8 | 9 | 0 | 0 | 0 | 30 |
| 22 | 9 | 9 | 7 | 7 | 8 | 9 | 0 | 0 | 0 | 30 |
| 23 | 9 | 9 | 9 | 9 | 8 | 9 | 0 | 0 | 0 | 30 |
| 39 | N | 9 | N | N | N | 9 | 0 | 0 | 0 | 30 |
| 40 | 9 | 9 | 7 | 7 | 7 | 9 | 0 | 0 | 0 | 30 |
| 43 | 9 | 9 | 7 | 9 | 8 | 9 | 0 | 0 | 0 | 30 |
| 65 | 9 | N | N | 9 | 9 | N | 0 | 0 | 0 | 30 |
| 66 | 9 | 9 | 9 | 9 | 9 | 9 | 0 | 0 | 0 | 30 |
| 67 | 9 | 9 | N | 7 | 7 | 9 | 0 | 0 | 0 | 30 |
| 68 | 8 | 8 | 7 | 7 | 8 | 9 | 0 | 0 | 0 | 30 |
| 69 | 9 | 9 | 9 | 9 | 9 | 9 | 0 | 0 | 0 | 30 |
| 70 | 9 | 9 | 9 | 9 | 9 | 9 | 0 | 0 | 0 | 30 |
| 71 | 9 | 9 | 9 | 9 | 9 | 9 | 0 | 0 | 0 | 30 |
| 72 | 9 | 9 | 9 | 7 | 8 | 9 | 0 | 0 | 0 | 30 |
| 73 | 9 | 9 | 9 | 8 | 8 | 9 | 0 | 0 | 0 | 30 |
| 74 | 9 | 9 | 9 | 9 | 8 | 9 | 0 | 0 | 0 | 30 |
| 75 | 9 | 9 | 9 | 9 | 9 | 9 | 0 | 0 | 0 | 30 |
| 76 | 9 | 9 | 9 | 9 | 8 | 9 | 0 | 0 | 0 | 30 |
| 77 | 9 | 9 | 7 | 8 | 8 | 9 | 0 | 0 | 0 | 30 |
| 78 | 9 | 9 | 8 | 9 | 8 | 9 | 0 | 0 | 0 | 30 |
| 79 | 9 | 9 | 8 | 9 | 9 | 9 | 0 | 0 | 0 | 30 |
| 80 | 9 | 9 | 9 | 9 | 8 | 9 | 0 | 0 | 0 | 30 |
| 81 | 9 | 9 | N | N | N | 9 | N | N | N | 30 |
| 83 | 9 | 9 | 9 | 9 | 9 | 9 | 0 | 0 | 0 | 30 |
| 84 | 9 | 9 | 9 | 9 | 9 | 9 | 0 | 0 | 0 | 30 |
| 85 | N | 9 | N | N | N | 9 | 0 | 0 | 0 | 30 |
| 86 | N | 9 | N | N | N | N | N | N | N | 30 |
| 87 | N | 8 | N | N | N | N | N | N | N | 30 |
| 88 | N | N | 9 | N | N | 9 | N | N | N | 30 |
| 89 | N | N | N | 9 | 9 | 9 | N | N | N | 30 |
| 94 | N | N | N | N | 8 | N | N | N | N | 30 |
| 96 | 9 | N | N | N | N | 8 | N | N | N | 30 |
| 100 | N | N | 8 | N | 9 | N | N | N | N | 30 |
| 111 | 9 | N | N | N | 8 | N | N | N | N | 30 |
| 120 | 9 | N | N | N | N | N | N | N | N | 30 |
| 121 | 9 | N | N | N | N | N | N | N | N | 30 |
| 122 | 9 | 8 | N | 8 | 9 | 8 | N | N | N | 30 |
| 123 | 9 | N | N | N | N | N | N | N | N | 30 |
| 125 | 9 | N | 9 | N | N | N | N | N | N | 30 |
| 126 | 9 | N | 9 | N | N | N | N | N | N | 30 |
| 127 | 9 | N | 9 | N | 8 | N | N | N | N | 30 |
| 128 | N | N | 9 | N | N | N | N | N | N | 30 |
| 129 | N | N | N | 8 | 9 | N | N | N | N | 30 |
| 130 | 9 | N | 9 | N | N | N | N | N | N | 30 |
| 131 | N | N | 9 | N | N | N | N | N | N | 30 |
| 132 | 9 | 9 | 9 | 9 | 9 | 9 | 0 | 0 | 0 | 30 |
| 133 | 9 | 9 | 9 | 9 | 9 | 9 | 0 | 0 | 0 | 30 |
| 134 | 9 | N | N | 8 | N | 8 | N | N | N | 30 |
| 135 | 9 | 9 | 9 | 9 | 9 | 9 | 0 | 0 | 0 | 30 |
| 136 | 9 | 9 | 9 | 9 | 9 | 9 | 0 | 0 | 0 | 30 |
| 137 | 9 | 9 | 9 | 8 | 9 | 9 | 0 | 0 | 0 | 30 |
| 138 | 9 | N | 9 | N | N | N | N | N | N | 30 |
| 139 | 9 | 9 | 9 | 9 | 9 | 9 | 0 | 0 | 0 | 30 |
| 140 | 9 | 9 | 9 | 8 | 9 | N | 0 | 0 | 0 | 30 |
| 141 | 9 | 9 | 9 | 9 | 8 | 8 | 0 | 0 | 0 | 30 |
| 142 | 9 | N | 9 | 9 | 9 | 9 | 0 | 0 | 0 | 30 |
| 143 | 9 | 9 | 9 | 8 | 9 | 9 | 0 | 0 | 0 | 30 |
| 144 | 9 | 9 | 9 | 8 | 9 | 9 | 0 | 0 | 0 | 30 |
| 145 | 9 | 9 | 9 | 9 | 9 | 9 | 0 | 0 | 0 | 30 |
| 146 | 9 | 9 | 9 | 8 | 9 | 9 | 0 | 0 | 0 | 30 |
| 147 | 9 | 9 | 9 | 8 | 9 | 9 | 0 | 0 | 0 | 30 |
| 148 | 9 | 9 | 9 | 8 | 9 | 9 | 0 | 0 | 0 | 30 |
| 149 | N | N | N | 8 | 8 | N | 0 | 0 | 0 | 30 |
| Control | 7 | 7 | 3 | 5 | 4 | 7 | 0 | 0 | 0 | 30 |
| compound | | | | | | | | | | |
| A | | | | | | | | | | |

Note:
N represents no data;
Control compound A:

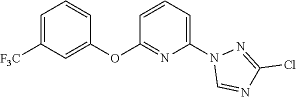 [SMILES: FC(F)(F)c1cccc(Oc2cccc(-n3cnc(Cl)n3)n2)c1]

Meanwhile, it has been found by experimentations on major weeds in wheat and rice fields that the compounds of the present invention generally have good weed control effects; in particular, it has been noticed that the compounds have extremely high activity on broad-leaf weeds and Cyperaceae that are resistant to ALS inhibitors, for example, Schoenoplectiella juncoides, Cyperus difformis, Descurainia sophia, Capsella bursa-pastoris, Lithospermum arvense, Galium spurium, Cyperus rotundus, etc., which have excellent commercial values.

(3) Safety evaluation on transplanted rice and control effect evaluation on weeds in rice fields:

After rice field soil was loaded into a 1/1,000,000 ha pot, the seeds of Echinochloa crusgalli, Schoenoplectiella juncoides, and Bidens tripartita were sowed and gently covered with soil. The water storage was kept at a depth of 3-4 cm thereafter. When the Echinochloa crusgalli, Schoenoplectiella juncoides, and Bidens tripartita reached their 0.5-leaf stages, aqueous dilutions of wettable powders or suspensions of the compounds of the present invention formulated according to common formulation methods were homogeneously dripped to the specified effective amount using pipettes for treatment.

In addition, after being loaded into the 1/1,000,000 ha pot, the rice field soil was leveled to keep the water storage at a depth of 3-4 cm. The rice (japonica rice) at 3-leaf stage was transplanted at a transplanting depth of 3 cm the next day. The compounds of the present invention were treated in the same manner as above on the 5^{th} day after the transplantation.

The developmental conditions of Echinochloa crusgalli, Schoenoplectiella juncoides, and Bidens tripartita on the 14^{th} day after the drug treatment and the developmental conditions of rice on the 21st day after the drug treatment were observed with the naked eye, respectively. The effects were evaluated according to the above-mentioned activity level criteria. Many compounds exhibited excellent activity and selectivity.

Note: Echinochloa crusgalli, Schoenoplectiella juncoides and Bidens tripartita seeds were collected from Heilongjiang, China and tested to be resistant to conventional doses of pyrazosulfuron-ethyl.

### (4) Pre-Emergence Sealing Experiment for Water-Seeding:

The seeds of Leptochloa chinensis, Digitaria sanguinalis, Monochoria vaginalis (sulfonylurea-resistant strain, collected from Hunan, China), Cyperus iria (sulfonylurea-resistant strain, collected from Jiangsu, China), Huaidao (rice cultivar), etc. were broadcasted on the surface of nutrition bowls filled with soil and fully covered by soil. The prepared nutrition bowls were then placed in a cultivation box, with water filled to a suitable height to keep the soil in the nutrition bowls moist. The test compounds of the present invention were individually dissolved with mixed solutions of acetone and Tween-80, then diluted into solution of a certain concentration using a certain amount of water and sprayed using a spray tower. The plants were placed on a greenhouse seedbed and cultured for 15 days, and the experimental results were then collected. The compounds were applied at 120, 60, 30 and 15 g a.i./ha in triplicate and the results were averaged. The effects were evaluated according to the above activity level criteria. Representative data were listed in Table 3.

TABLE 3
Results of pre-emergence sealing experiment for water-seeding

| | Leptochloa | Digitaria | Monochoria | | Dosage |
|---|---|---|---|---|---|
| Compound No. | chinensis | sanguinalis | vaginalis | Cyperus iria | (g a.i./ha) |
| 8 | 9 | 9 | 9 | 9 | 15 |
| 16 | 9 | 9 | 9 | 9 | 15 |
| 23 | 9 | 9 | 9 | 9 | 15 |
| Bensulfuron-methyl | 3 | 4 | 2 | 5 | 30 |

### (5) Activity Assay of Compositions:

The required active ingredient B was purchased from a reagent company or a technical material manufacturer or synthesized by a conventional method. The technical materials were all dissolved with acetone solvents and diluted with aqueous solution of 0.1% Tween-80 emulsifier, which should be used immediately after dilution.

### (A) Soil Sealing Treatment(S):

Weeds were cultivated in a controllable sunlight greenhouse at 20 to 30° C., in natural light, and with relative humidity of 57% to 72%. The soil was loam with an organic matter content of 1.63%, a pH value of 7.1, alkali-hydrolyzable nitrogen of 84.3 mg/kg, rapidly available phosphorus of 38.5 mg/kg, and rapidly available potassium of 82.1 mg/kg. The test soil was quantitatively filled to ¾ of the pot, and then watered from the bottom of the pot to completely wet the soil to saturation. The test weed seeds were accelerated to just germinate, and then evenly and quantitatively broadcasted on the soil surface, covered with 0.5-1 cm soil according to the sizes of the seeds, and ready for use 72 hours after sowing.

Each treatment was repeated 4 times. Four pots with 20 weed seeds sowed per pot were treated each time.

The well-sowed test materials were evenly placed on a platform with an area of 0.5 m², and foliage spraying was carried out by the 3WP-2000-type walking spray tower at a dosage of 450 kg/ha and a spray pressure of 0.3 MPa. After all the solution was sprayed, the air valve was closed. And 30 seconds later, the door of the spray tower was opened, and the nutritional bowl was taken out. Next, the air valve was opened, and the spray tube was cleaned by spraying 50 ml of water.

### (B) Post-Emergence Spray Treatment of Stems and Leaves (F):

Descurainia sophia weeds were cultivated by a pot culture method. A 180×140 mm plastic nutritional bowl, containing air-dried and sieved topsoil (taking up 4/5 of the bowl) collected from the field, was placed in an enamel pan, wherein the soil had an initial moisture content of 20%. Weed seeds with plump and uniform grains were selected, treated with 30% hydrogen peroxide for 10 min, and accelerated to germinate at 15-20° C. in a biochemical incubator (in darkness). The weed seeds that had just germinated were evenly placed on the surface of the soil and then covered with 0.5-1 cm soil according to the sizes of seeds.

The culture was carried out in a controllable sunlight greenhouse at 20-30° C., in natural light, and with relative humidity of 57%-72%. The soil was loam with an organic matter content of 1.63%, a pH value of 7.1, alkali-hydrolyzable nitrogen of 84.3 mg/kg, rapidly available phosphorus of 38.5 mg/kg, and rapidly available potassium of 82.1 mg/kg.

Each treatment was repeated 4 times. Three pots with 20 weed seeds sowed per pot were treated each time.

The agents were used once in total in the experiment. When at the stage of 1.5-2 leaves, the weeds were thinned out to maintain 10 weeds per pot. Thirty weeds were kept in each treatment, and then continued to be cultured until the 10-leaf stage for further treatment.

The well-cultured test materials were evenly placed on a platform with an area of 0.5 m², and foliage spraying was carried out by the 3WP-2000-type walking spray tower at a dosage of 450 kg/ha and a spray pressure of 0.3 MPa. After all the solution was sprayed, the air valve was closed, and 30 seconds later, the door of the spray tower was opened, and the nutritional bowl was taken out. Next, the air valve was opened, and the spray tube was cleaned by spraying 50 ml of water. The test materials were transferred to and routinely cultured in a greenhouse after the treatment.

### (C) Data Investigation and Statistical Analysis

A method for investigating absolute numbers was employed. Whole seedlings of survival weeds were cut off along the soil surface with a blade, and the fresh weight of the weeds was weighed using an analytical balance. For dead weeds, the fresh weight thereof was zero.

The investigation was performed 21 days after the treatment for once in total.

The theoretical fresh weight inhibition rate of a mixed combination of each treatment was calculated by the Gowing method (E0=X+Y−X*Y/100). Then compared with the measured inhibition rate (E), the type of the combined effect of the combination of the two on weeds was evaluated, which was a synergistic effect when E−E0>10%; an antagonistic effect when E−E0<−10%; an additional effect when −10%≤E−E0≤10%. An optimal ratio was determined according to factors such as the actual control effects, characteristics of herbicides, balance of the formulation, etc. In the formula, X represents the fresh weight inhibition rate of the active ingredient A in a dosage of P; Y represents the fresh weight inhibition rate of the active ingredient B in a dosage of Q. The statistical results are shown in Table 4.

TABLE 4
Evaluation of actual control effect and combined effect on weeds of Compound 16
mixtures

| | | | | Control | Control | | | |
|---|---|---|---|---|---|---|---|---|
| | | | | effect of | effect of | | Theo- | |
| | | | | A applied | B applied | Actual | retical | |
| | | | | alone | alone | control | control | |
| | | | | in a | in a | effect | effect | |
| | | Treat- | | corre- | corre- | of A + | of A + | |
| Component | | ment | Dosage | sponding | sponding | B (%), | B (%), | E-E0 |
| (A + B) | Weed | F/S | g a.i./ha | dose (%) | dose (%) | E | E0 | (%) |
| Compound 16 + | Amaranthus | S | 7.5 + | 57.3 | 40.2 | 85.6 | 74.5 | 11.1 |
| Acetochlor | retroflexus | | 150 | | | | | |
| | | | 7.5 + | 57.3 | 58.3 | 93.8 | 82.2 | 11.6 |
| | | | 300 | | | | | |
| Compound 16 + | Amaranthus | S | 7.5 + | 57.3 | 41.4 | 87.9 | 75.0 | 12.9 |
| S-metolachlor | retroflexus | | 150 | | | | | |
| | | | 7.5 + | 57.3 | 64.6 | 98.4 | 84.9 | 13.5 |
| | | | 300 | | | | | |
| Compound 16 + | Eclipta | S | 7.5 + 75 | 46.4 | 38.4 | 80.4 | 67.0 | 13.4 |
| Pretilachlor | prostrata | | 7.5 + | 46.4 | 68.3 | 94.8 | 83.0 | 11.8 |
| | | | 150 | | | | | |
| Compound 16 + | Eclipta | S | 7.5 + | 46.4 | 37.2 | 83.3 | 66.3 | 17.0 |
| Butachlor | prostrata | | 150 | | | | | |
| | | | 7.5 + | 46.4 | 66.8 | 96.8 | 82.2 | 14.6 |
| | | | 300 | | | | | |
| Compound 16 + | Eclipta | S | 7.5 + | 46.4 | 40.6 | 84.3 | 68.2 | 16.1 |
| Mefenacet | prostrata | | 150 | | | | | |
| | | | 7.5 + | 46.4 | 69.7 | 97.2 | 83.8 | 13.4 |
| | | | 300 | | | | | |
| Compound 16 + | Eclipta | S | 7.5 + 75 | 46.4 | 14.2 | 65.1 | 54.0 | 11.1 |
| Anilofos | prostrata | | 7.5 + | 46.4 | 23.6 | 72.3 | 59.0 | 13.3 |
| | | | 150 | | | | | |
| Compound 16 + | Lolium | S | 15 + 30 | 37.8 | 40.6 | 74.2 | 63.1 | 11.1 |
| Pyroxasulfone | multiflorum | | 15 + 60 | 37.8 | 62.7 | 89.3 | 76.8 | 12.5 |
| Compound 16 + | Lolium | S | 15 + 30 | 37.8 | 46.2 | 78.2 | 66.5 | 11.7 |
| Flufenacet | multiflorum | | 15 + 60 | 37.8 | 68.5 | 92.3 | 80.4 | 11.9 |
| Compound 16 + | Eclipta | S | 7.5 + | 46.4 | 45.5 | 82.2 | 70.8 | 11.4 |
| Pendimethalin | prostrata | | 150 | | | | | |
| | | | 7.5 + | 46.4 | 63.2 | 91.3 | 80.3 | 11.0 |
| | | | 300 | | | | | |
| Compound 16 + | Eleocharis | S | 15 + 150 | 36.3 | 40.6 | 82.1 | 62.2 | 19.9 |
| Metribuzin | yokoscensis | | 15 + 300 | 36.3 | 58.3 | 92.5 | 73.4 | 19.1 |
| Compound 16 + | Eleocharis | S | 15 + 150 | 36.3 | 42.1 | 80.2 | 63.1 | 17.1 |
| Prometryn | yokoscensis | | 15 + 300 | 36.3 | 56.9 | 89.4 | 72.5 | 16.9 |
| Compound 16 + | Chloris | S | 15 + 300 | 37.2 | 40.1 | 82.0 | 62.4 | 19.6 |
| Atrazine | virgata | | 15 + 600 | 37.2 | 58.9 | 93.9 | 74.2 | 19.7 |
| Compound 16 + | Chloris | S | 15 + 225 | 37.2 | 35.5 | 78.8 | 59.5 | 19.3 |
| Terbuthylazine | virgata | | 15 + 450 | 37.2 | 52.7 | 88.3 | 70.3 | 18.0 |
| Compound 16 + | Lolium | S | 15 + 375 | 41.1 | 37.1 | 82.4 | 63.0 | 19.4 |
| Isoproturon | temulentum | | 15 + 750 | 41.1 | 58.2 | 92.7 | 75.4 | 17.3 |
| Compound 16 + | Lolium | S | 15 + 375 | 41.1 | 34.3 | 80.4 | 61.3 | 19.1 |
| Chlorotoluron | temulentum | | 15 + 750 | 41.1 | 59.3 | 93.1 | 76.0 | 17.1 |
| Compound 16 + | Commelina | S | 15 + 15 | 26.8 | 35.0 | 67.2 | 52.4 | 14.8 |
| Flumioxazin | communis | | 15 + 30 | 26.8 | 50.6 | 78.5 | 63.8 | 14.7 |
| Compound 16 + | Commelina | S | 15 + 30 | 26.8 | 28.5 | 60.8 | 47.7 | 13.1 |
| | communis | | 15 + 60 | 26.8 | 46.1 | 73.2 | 60.5 | 12.7 |
| 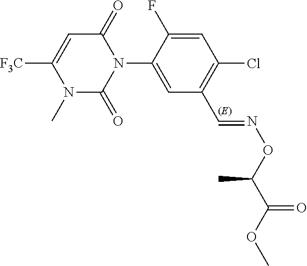 [SMILES: COC(=O)[C@@H](C)O/N=C/c1cc(-n2c(=O)cc(C(F)(F)F)n(C)c2=O)c(F)cc1Cl] | | | | | | | | |
| Compound 16 + | Solanum | S | 15 + 30 | 34.2 | 45.8 | 76.1 | 64.3 | 11.8 |
| Oxadiazon | nigrum | | 15 + 60 | 34.2 | 53.5 | 83.6 | 69.4 | 14.2 |
| Compound 16 + | Solanum | S | 15 + 45 | 34.2 | 51.2 | 79.2 | 67.9 | 11.3 |
| Oxadiargyl | nigrum | | 15 + 90 | 34.2 | 64.7 | 87.6 | 76.8 | 10.8 |
| Compound 16 + | Amaranthus | S | 7.5 + 15 | 57.3 | 36.7 | 84.6 | 73.0 | 11.6 |
| Isoxaflutole | retroflexus | | 7.5 + 30 | 57.3 | 50.3 | 92.7 | 78.8 | 13.9 |
| Compound 16 + | Amaranthus | S | 7.5 + 15 | 57.3 | 13.5 | 74.9 | 63.1 | 11.8 |
| Fenpyrazone | retroflexus | | 7.5 + 30 | 57.3 | 33.7 | 82.1 | 71.7 | 10.4 |
| Compound 16 + | Ammannia | S | 7.5 + 15 | 60.6 | 26.1 | 82.6 | 70.9 | 11.7 |
| | arenaria | | 7.5 + 30 | 60.6 | 43.8 | 90.5 | 77.9 | 12.6 |
| 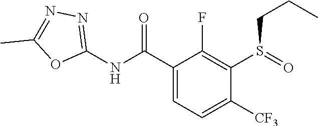 [SMILES: CCC[S@@](=O)c1c(C(F)(F)F)ccc(C(=O)Nc2nnc(C)o2)c1F] | | | | | | | | |
| Compound 16 + | Ammannia | S | 7.5 + 7.5 | 60.6 | 32.9 | 86.3 | 73.6 | 12.7 |
| Tefuryltrione | arenaria | | 7.5 + 15 | 60.6 | 55.2 | 95.7 | 82.3 | 13.4 |
| Compound 16 + | Lolium | S | 15 + 75 | 37.8 | 43.7 | 77.1 | 65.0 | 12.1 |
| | multiflorum | | 15 + 150 | 37.8 | 61.0 | 93.3 | 75.7 | 17.6 |
| 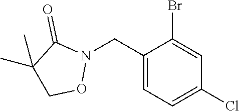 [SMILES: CC1(C)CON(Cc2ccc(Cl)cc2Br)C1=O] | | | | | | | | |
| Compound 16 + | Lolium | S | 15 + 30 | 37.8 | 55.2 | 84.5 | 72.1 | 12.4 |
| | multiflorum | | 15 + 60 | 37.8 | 70.4 | 95.8 | 81.6 | 14.2 |
| 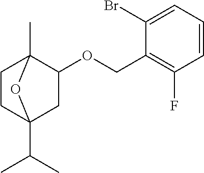 [SMILES: CC(C)C12CCC(C)(O1)C(OCc1c(F)cccc1Br)C2] | | | | | | | | |
| Compound 16 + | Silene | S | 7.5 + 75 | 40.3 | 48.0 | 80.2 | 69.0 | 11.2 |
| | conoidea | | 7.5 + 150 | 40.3 | 54.2 | 88.4 | 72.7 | 15.7 |
| 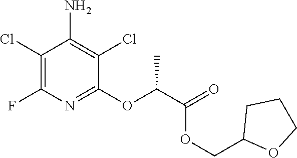 [SMILES: C[C@@H](Oc1nc(F)c(Cl)c(N)c1Cl)C(=O)OCC1CCCO1] | | | | | | | | |
| Compound 16 + | Silene | S | 7.5 + 15 | 40.3 | 43.7 | 78.0 | 66.4 | 11.6 |
| Bensulfuron- | conoidea | | 7.5 + 30 | 40.3 | 60.5 | 87.6 | 76.4 | 11.2 |
| methyl | | | | | | | | |
| Compound 16 + | Silene | S | 7.5 + 15 | 40.3 | 50.4 | 83.2 | 70.4 | 12.8 |
| Pyrazosulfuron- | conoidea | | 7.5 + 30 | 40.3 | 65.6 | 91.5 | 79.5 | 12.0 |
| ethyl | | | | | | | | |
| Compound 16 + | Silene | S | 7.5 + 30 | 40.3 | 47.5 | 80.3 | 68.7 | 11.6 |
| Thifensulfuron- | conoidea | | 7.5 + 60 | 40.3 | 62.1 | 90.7 | 77.4 | 13.3 |
| methyl | | | | | | | | |
| Compound 16 + | Eclipta | S | 7.5 + | 46.4 | 35.1 | 79.0 | 65.2 | 13.8 |
| Fentrazamide | prostrata | | 22.5 | | | | | |
| | | | 7.5 + 45 | 46.4 | 48.9 | 84.7 | 72.6 | 12.1 |
| Compound 16 + | Eclipta | S | 7.5 + | 46.4 | 47.0 | 84.1 | 71.6 | 12.5 |
| Butralin | prostrata | | 150 | | | | | |
| | | | 7.5 + | 46.4 | 65.3 | 95.0 | 81.4 | 13.6 |
| | | | 300 | | | | | |
| Compound 16 + | Lolium | S | 15 + 300 | 37.8 | 46.5 | 79.8 | 66.7 | 13.1 |
| Prosulfocarb | multiflorum | | 15 + 600 | 37.8 | 64.2 | 93.4 | 77.7 | 15.7 |
| Compound 16 + | Solanum | S | 15 + 15 | 34.2 | 48.5 | 79.1 | 66.1 | 13.0 |
| Sulfentrazone | nigrum | | 15 + 30 | 34.2 | 60.0 | 88.5 | 73.7 | 14.8 |
| Compound 16 + | Eclipta | S | 7.5 + | 46.4 | 47.0 | 84.7 | 71.6 | 13.1 |
| | prostrata | | 37.5 | | | | | |
| Pyraclonil | | | 7.5 + 75 | 46.4 | 65.8 | 95.5 | 81.7 | 13.8 |
| Compound 16 + | Eclipta | S | 7.5 + 30 | 46.4 | 40.5 | 80.0 | 68.1 | 11.9 |
| Pentoxazone | prostrata | | 7.5 + 60 | 46.4 | 61.4 | 93.3 | 79.3 | 14.0 |
| Compound 16 + | Eclipta | S | 7.5 + 7.5 | 46.4 | 50.8 | 90.7 | 73.6 | 17.1 |
| Saflufenacil | prostrata | | 7.5 + 15 | 46.4 | 69.6 | 98.1 | 83.7 | 14.4 |
| Compound 16 + | Silene | S | 7.5 + 30 | 40.3 | 44.4 | 82.9 | 66.8 | 16.1 |
| Trifludimoxazin | conoidea | | 7.5 + 60 | 40.3 | 66.7 | 95.1 | 80.1 | 15.0 |
| Compound 16 + | Eclipta | S | 7.5 + | 46.4 | 49.2 | 89.1 | 72.8 | 16.3 |
| | prostrata | | 22.5 | | | | | |
| Benzobicyclon | | | 7.5 + 45 | 46.4 | 74.0 | 98.7 | 86.1 | 12.6 |
| Compound 16 + | Lolium | S | 15 + 75 | 37.8 | 50.2 | 83.6 | 69.0 | 14.6 |
| Bixlozone | multiflorum | | 15 + 150 | 37.8 | 70.0 | 95.6 | 81.3 | 14.3 |
| Compound 16 + | Eclipta | S | 7.5 + 7.5 | 46.4 | 51.5 | 85.2 | 74.0 | 11.2 |
| Cinmethylin | prostrata | | 7.5 + 15 | 46.4 | 72.0 | 99.4 | 85.0 | 14.4 |
| Compound 16 + | Eclipta | S | 7.5 + 7.5 | 46.4 | 38.9 | 79.9 | 67.3 | 12.6 |
| Oxaziclomefone | prostrata | | 7.5 + 15 | 46.4 | 56.8 | 90.0 | 76.8 | 13.2 |
| Compound 16 + | Eclipta | S | 7.5 + 15 | 46.4 | 51.6 | 88.8 | 74.1 | 14.7 |
| Metazosulfuron | prostrata | | 7.5 + 30 | 46.4 | 69.2 | 94.7 | 83.5 | 11.2 |
| Compound 16 + | Eclipta | S | 7.5 + 7.5 | 46.4 | 42.8 | 84.0 | 69.3 | 14.7 |
| Propyrisulfuron | prostrata | | 7.5 + 15 | 46.4 | 63.0 | 95.5 | 80.2 | 15.3 |
| Compound 16 + | Commelina | S | 15 + 15 | 26.8 | 45.4 | 70.3 | 60.0 | 10.3 |
| Thiencarbazone- | communis | | 15 + 30 | 26.8 | 56.0 | 80.9 | 67.8 | 13.1 |
| methyl | | | | | | | | |
| Compound 16 + | Eclipta | S | 7.5 + 7.5 | 46.4 | 41.1 | 79.6 | 68.4 | 11.2 |
| Triafamone | prostrata | | 7.5 + 15 | 46.4 | 68.2 | 97.4 | 83.0 | 14.4 |
| Compound 16 + | Eclipta | S | 7.5 + 7.5 | 46.4 | 54.4 | 89.2 | 75.6 | 13.6 |
| Pyrimisulfan | prostrata | | 7.5 + 15 | 46.4 | 73.7 | 98.0 | 85.9 | 12.1 |
| Compound 16 + | Monochoria | S | 1.5 + 7.5 | 62.5 | 43.9 | 90.5 | 79.0 | 11.5 |
| Tetflupyrolimet | vaginalis | | 1.5 + 15 | 62.5 | 68.1 | 99.2 | 88.0 | 11.2 |
| Compound 16 + | Commelina | S | 15 + 30 | 26.8 | 35.0 | 63.8 | 52.4 | 11.4 |
| 3-[2-chloro-5- | communis | | 15 + 60 | 26.8 | 47.3 | 75.7 | 61.4 | 14.3 |
| [3,6-dihydro-3- | | | | | | | | |
| methyl-2,6- | | | | | | | | |
| dioxo-4- | | | | | | | | |
| (trifluoromethyl)- | | | | | | | | |
| 1(2H)- | | | | | | | | |
| pyrimidinyl]-4- | | | | | | | | |
| fluorophenyl]- | | | | | | | | |
| 4,5-dihydro-5- | | | | | | | | |
| methyl-5- | | | | | | | | |
| isoxazolecarboxy | | | | | | | | |
| lic acid ethyl | | | | | | | | |
| ester | | | | | | | | |
| Compound 16+ | Commelina | S | 15 + 30 | 26.8 | 38.8 | 65.5 | 55.2 | 10.3 |
| Epyrifenacil | communis | | 15 + 60 | 26.8 | 50.5 | 77.0 | 63.8 | 13.2 |
| Compound 16 + | Commelina | S | 15 + 30 | 26.8 | 48.2 | 75.2 | 62.1 | 13.1 |
| Tiafenacil | communis | | 15 + 60 | 26.8 | 61.4 | 84.4 | 71.7 | 12.7 |
| Compound 16 + | Descurainia | F | 15 + 15 | 47.0 | 55.4 | 89.7 | 76.4 | 13.3 |
| Bipyrazone | sophia | | 15 + 30 | 47.0 | 78.9 | 100.0 | 88.8 | 11.2 |
| Compound 16 + | Eclipta | S | 7.5 + 150 | 46.4 | 45.7 | 87.5 | 70.9 | 16.6 |
| Trifluralin | prostrata | | 7.5 + 300 | 46.4 | 67.5 | 93.6 | 82.6 | 11.0 |
| Compound 16 + | Lolium | S | 15 + 150 | 37.8 | 38.4 | 73.3 | 61.7 | 11.6 |
| | multiflorum | | 15 + 300 | 37.8 | 53.7 | 85.4 | 71.2 | 14.2 |
| 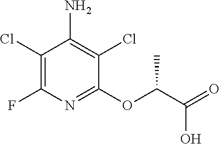 [SMILES: C[C@@H](Oc1nc(F)c(Cl)c(N)c1Cl)C(=O)O] | | | | | | | | |
| Compound 16 + | Descurainia | F | 15 + 30 | 47.0 | 40.5 | 83.9 | 68.5 | 15.4 |
| Clopyralid | sophia | | 15 + 60 | 47.0 | 58.8 | 94.4 | 78.2 | 16.2 |
| Compound 16 + | Descurainia | F | 15 + 150 | 47.0 | 44.7 | 85.8 | 70.7 | 15.1 |
| 2,4-D- Choline | sophia | | 15 + 300 | 47.0 | 60.0 | 94.0 | 78.8 | 15.2 |
| Compound 16 + | Descurainia | F | 15 + 37.5 | 47.0 | 39.6 | 81.3 | 68.0 | 13.3 |
| Dicamba | sophia | | 15 + 75 | 47.0 | 50.8 | 90.1 | 73.9 | 16.2 |
| Compound 16 + | Descurainia | F | 15 + 6 | 47.0 | 26.8 | 73.1 | 61.2 | 11.9 |
| | sophia | | 15 + 12 | 47.0 | 37.7 | 79.4 | 67.0 | 12.4 |
| 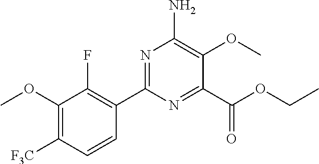 [SMILES: CCOC(=O)c1nc(-c2ccc(C(F)(F)F)c(OC)c2F)nc(N)c1OC] | | | | | | | | |
| Compound 16 + | Descurainia | F | 15 + 3 | 47.0 | 42.0 | 85.2 | 69.3 | 15.9 |
| Halauxifen- | sophia | | 15 + 6 | 47.0 | 67.4 | 98.1 | 82.7 | 15.4 |
| methyl | | | | | | | | |
| Compound 16 + | Descurainia | F | 15 + 75 | 47.0 | 56.6 | 90.7 | 77.0 | 13.7 |
| Glyphosate | sophia | | 15 + 150 | 47.0 | 75.8 | 100.0 | 87.2 | 12.8 |
| Compound 16 + | Descurainia | F | 15 + 60 | 47.0 | 50.7 | 86.8 | 73.9 | 12.9 |
| Glufosinate- | sophia | | 15 + 120 | 47.0 | 71.5 | 98.5 | 84.9 | 13.6 |
| ammonium | | | | | | | | |
| Compound 16 + | Descurainia | F | 15 + 30 | 47.0 | 46.9 | 84.3 | 71.9 | 12.4 |
| Paraquat | sophia | | 15 + 60 | 47.0 | 68.0 | 95.5 | 83.0 | 12.5 |
| dichloride | | | | | | | | |
| Compound 16 + | Descurainia | F | 15 + 60 | 47.0 | 42.2 | 82.8 | 69.4 | 13.4 |
| Diquat dibromide | sophia | | 15 + 120 | 47.0 | 66.6 | 95.0 | 82.3 | 12.7 |
| monohydrate | | | | | | | | |
| Compound 16 + | Descurainia | S | 15 + 150 | 62.5 | 38.6 | 89.2 | 77.0 | 12.2 |
| Napropamide | sophia | | 15 + 300 | 62.5 | 55.7 | 98.4 | 83.4 | 15.0 |

Meanwhile, it has been found by extensive experimentations that many of the compounds of the present invention and compositions thereof have good selectivity to gramineae grasses such as zoysia grass, bermuda grass, tall fescue, bluegrass, ryegrass, seashore paspalum, etc., and can prevent and control many key gramineous weeds and broad-leaf weeds. Tests on sugarcane, soybean, cotton, oil sunflower, potato, fruit trees, vegetables and the like using different herbicide application methods also demonstrated excellent selectivity and commercial values.

1. A triazole compound is as shown in the general formula I: 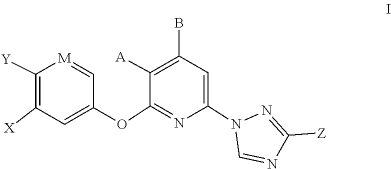
wherein, M represents N or CH;
X represents halogen, alkyl, haloalkyl, cyano, or haloalkoxy;
Y represents hydrogen or halogen;
Z represents hydrogen, halogen, cyano, or unsubstituted or halogen-substituted alkyl, alkenyl, alkynyl, cycloalkyl, alkoxy, alkenyloxy, alkynyloxy, alkoxyalkyl, alkylthio, alkylsulfoxide, or alkylsulfonyl;
A and B each independently represent hydrogen, halogen, cyano, alkyl, cycloalkyl, alkoxy, alkylthio, alkylthioalkyl, or alkoxyalkyl, and also, A and B are not simultaneously hydrogen.

2. The triazole compound according to claim 1, wherein
X represents halogen, C1-C8 alkyl, halo C1-C8 alkyl, cyano, or halo C1-C8 alkoxy;
Z represents hydrogen, halogen, cyano, or unsubstituted or halogen-substituted C1-C8 alkyl, C2-C8 alkenyl, C2-C8 alkynyl, C3-C8 cycloalkyl, C1-C8 alkoxy, C2-C8 alkenyloxy, C2-C8 alkynyloxy, C1-C8 alkoxy C1-C8 alkyl, C1-C8 alkylthio, C1-C8 alkylsulfoxide, or C1-C8 alkylsulfonyl;
A and B each independently represent hydrogen, halogen, cyano, C1-C8 alkyl, C3-C8 cycloalkyl, C1-C8 alkoxy, C1-C8 alkylthio, C1-C8 alkylthio C1-C8 alkyl, or C1-C8 alkoxy C1-C8 alkyl.

3. The triazole compound according to claim 1, wherein
X represents halogen, C1-C6 alkyl, halo C1-C6 alkyl, cyano, or halo C1-C6 alkoxy;
Z represents hydrogen, halogen, cyano, or unsubstituted or halogen-substituted C1-C6 alkyl, C2-C6 alkenyl, C2-C6 alkynyl, C3-C6 cycloalkyl, C1-C6 alkoxy, C2-C6 alkenyloxy, C2-C6 alkynyloxy, C1-C6 alkoxy C1-C6 alkyl, C1-C6 alkylthio, C1-C6 alkylsulfoxide, or C1-C6 alkylsulfonyl;
A and B each independently represent hydrogen, halogen, cyano, C1-C6 alkyl, C3-C6 cycloalkyl, C1-C6 alkoxy, C1-C6 alkylthio, C1-C6 alkylthio C1-C6 alkyl, or C1-C6 alkoxy C1-C6 alkyl.

4. The triazole compound according to claim 1, wherein the compound is selected from the group consisting of:
I

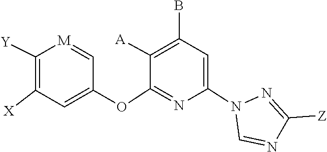

| No. | A | B | X | Y | Z | M |
|---|---|---|---|---|---|---|
| 1 | CH₃ | CH₃ | CF₃ | H | CF₃ | N |
| 2 | H | CN | CF₃ | H | CF₃ | CH |
| 3 | H | OCH₃ | CF₃ | H | CF₃ | CH |
| 4 | CH₃ | CH₃ | CF₃ | H | CF₃ | CH |
| 5 | H | CH₃ | CF₃ | H | CF₃ | CH |
| 6 | H | CH₃ | CF₃ | H | CF₃ | N |
| 7 | C1 | H | CF₃ | H | CF₃ | CH |
| 8 | CH₃ | H | CF₃ | H | CF₃ | CH |
| 9 | CH₃ | H | OCF₃ | H | CF₃ | CH |
| 10 | CH₃ | | CF₃ | F | CF₃ | CH |
| 11 | CH₃ | H | CF₃ | H | CF₃ | N |
| 12 | CH₃ | H | CHF₂ | H | CF₃ | CH |
| 13 | CH₃ | H | CF₃ | H | 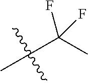 [SMILES: *C(C)(F)F] | CH |
| 14 | CH₃ | H | CF₃ | H | 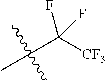 [SMILES: *C(F)(F)C(F)(F)F] | CH |
| 15 | CH₃ | H | CF₃ | H | 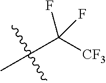 [SMILES: *C(F)(F)C(F)(F)F] | N |
| 16 | CH₂CH₃ | H | CF₃ | H | CF₃ | CH |
| 17 | CH₂CH₃ | H | CF₃ | H | CF₃ | N |
| 18 | CH₂CH₃ | H | CF₃ | H | 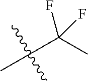 [SMILES: *C(C)(F)F] | CH |
| 19 | CH₂CH₃ | H | CF₃ | H | 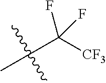 [SMILES: *C(F)(F)C(F)(F)F] | CH |
| 20 | CH₂CH₃ | H | 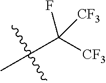 [SMILES: *C(F)(C(F)(F)F)C(F)(F)F] | H | CF₃ | CH |
| 21 | 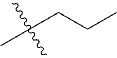 [SMILES: *CCC] | H | CF₃ | H | CF₃ | CH |
| 22 | 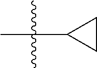 [SMILES: *C1CC1] | H | CF₃ | H | CF₃ | CH |
| 23 | 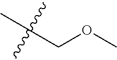 [SMILES: *COC] | H | CF₃ | H | CF₃ | CH |
| 24 | F | H | CF₃ | H | CF₃ | CH |
| 25 | CN | H | CF₃ | H | CF₃ | CH |
| 26 | OCH₃ | H | CF₃ | H | CF₃ | CH |
| 27 | OCH₂CH₃ | H | CF₃ | H | CF₃ | CH |
| 28 | 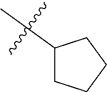 [SMILES: *C1CCCC1] | H | CF₃ | H | CF₃ | CH |
| 29 | 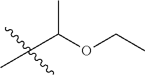 [SMILES: *C(C)OCC] | H | CF₃ | H | CF₃ | CH |
| 30 | CH₃ | F | CF₃ | H | CF₃ | CH |
| 31 | CH₃ | Cl | CF₃ | H | CF₃ | CH |
| 32 | CH₃ | CH₂CH₃ | CF₃ | H | CF₃ | CH |
| 33 | CH₃ | 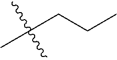 [SMILES: *CCC] | CF₃ | H | CF₃ | CH |
| 34 | CH₃ | 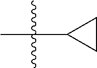 [SMILES: *C1CC1] | CF₃ | H | CF₃ | CH |
| 35 | CH₃ | 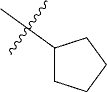 [SMILES: *C1CCCC1] | CF₃ | H | CF₃ | CH |
| 36 | CH₃ | 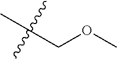 [SMILES: *COC] | CF₃ | H | CF₃ | CH |
| 37 | CH₃ | 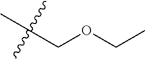 [SMILES: *COCC] | CF₃ | H | CF₃ | CH |
| 38 | CH₃ | 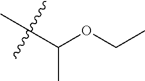 [SMILES: *C(C)OCC] | CF₃ | H | CF₃ | CH |
| 39 | CH₃ | H | F | H | CF₃ | CH |
| 40 | CH₃ | H | Cl | H | CF₃ | CH |
| 41 | CH₃ | H | CH₃ | H | CF₃ | CH |
| 42 | CH₃ | H | CH₂CH₃ | H | CF₃ | CH |
| 43 | CH₃ | H | OCHF₂ | H | CF₃ | CH |
| 44 | CH₃ | H | OCF₃ | H | CF₃ | ZI |
| 45 | CH₃ | H | CF₃ | Cl | CF₃ | CH |
| 46 | CH₃ | H | CF₃ | Br | CF₃ | CH |
| 47 | CH₃ | H | CF₃ | H | H | CH |
| 48 | CH₃ | H | CF₃ | H | F | CH |
| 49 | CH₃ | H | CF₃ | H | C1 | CH |
| 50 | CH₃ | H | CF₃ | H | CN | CH |
| 51 | CH₃ | H | CF₃ | H | CH₃ | CH |
| 52 | CH₃ | H | CF₃ | H | CH₂CH₃ | CH |
| 53 | CH₃ | H | CF₃ | H | 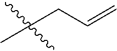 [SMILES: *CC=C] | CH |
| 54 | CH₃ | H | CF₃ | H | 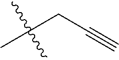 [SMILES: *CC#C] | CH |
| 55 | CH₃ | H | CF₃ | H | 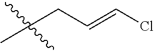 [SMILES: *C/C=C/Cl] | CH |
| 56 | CH₃ | H | CF₃ | H | 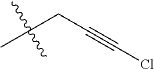 [SMILES: *CC#CCl] | CH |
| 57 | CH₃ | H | CF₃ | H | OCH₃ | CH |
| 58 | CH₃ | H | CF₃ | H | OCF₃ | CH |
| 59 | CH₃ | H | CF₃ | H | OCHF₂ | CH |
| 60 | CH₃ | H | CF₃ | H | 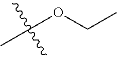 [SMILES: *OCC] | CH |
| 61 | CH₃ | H | CF₃ | H | 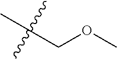 [SMILES: *COC] | CH |
| 62 | CH₃ | H | CF₃ | H | SO₂CH₃ | CH |
| 63 | CH₃ | H | CF₃ | H | 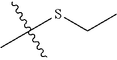 [SMILES: *SCC] | CH |
| 64 | CH₃ | H | CF₃ | H | 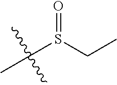 [SMILES: *S(=O)CC] | CH |
| 65 | CH₃ | H | OCF₃ | H | 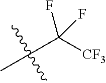 [SMILES: *C(F)(F)C(F)(F)F] | CH |
| 66 | CH₃ | H | OCF₃ | F | CF₃ | CH |
| 67 | H | OCH₃ | OCF₃ | H | CHF₂ | CH |
| 68 | H | 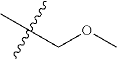 [SMILES: *COC] | CF₃ | H | CF | CH |
| 69 | CH₂CH₃ | H | CF₂CH₃ | H | CF₃ | CH |
| 70 | CH₃ | H | CF₃ | F | CHF₂ | CH |
| 71 | CH₂CH₃ | H | OCF₃ | H | CF₃ | CH |
| 72 | CH₂CH₃ | H | CF₃ | H | CHF₂ | CH |
| 73 | CH₂CH₃ | H | OCF₃ | H | CHF₂ | CH |
| 74 | CH₂CH₃ | H | CF₃ | H | CHF₂ | N |
| 75 | CH₃ | H | OCHF₂ | F | CF₃ | CH |
| 76 | H | OCH₃ | CF₃ | F | CF₃ | CH |
| 77 | 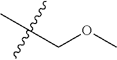 [SMILES: *COC] | H | OCF₃ | H | 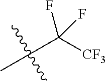 [SMILES: *C(F)(F)C(F)(F)F] | CH |
| 78 | H | OCH₃ | OCF₃ | H | CF₃ | CH |
| 79 | CH₃ | H | OCHF₂ | H | CHF₂ | CH |
| 80 | CH₃ | H | OCF₃ | F | CHF₂ | CH |
| 81 | CH₃ | H | F | H | CHF₂ | CH |
| 82 | CH₃ | H | CH₃ | H | CHF₂ | CH |
| 83 | CH₃ | H | OCF₃ | H | CHF₂ | CH |
| 84 | CH₃ | H | CHF₂ | H | CHF₂ | CH |
| 85 | CH₂CH₃ | H | CH2_{F} | H | CF₃ | CH |
| 86 | H | SCH₃ | CF₃ | H | CF₃ | CH |
| 87 | H | SCH₃ | OCF₃ | H | CF₃ | CH |
| 88 | CH₃ | H | CN | H | CF₃ | CH |
| 89 | CH₂CH₃ | H | CN | H | CF₃ | CH |
| 90 | 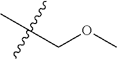 [SMILES: *COC] | H | CN | H | CF₃ | CH |
| 91 | 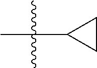 [SMILES: *C1CC1] | H | CN | H | CF₃ | CH |
| 92 | H | CH₃ | CN | H | CF₃ | CH |
| 93 | H | OCH₃ | CN | H | CF₃ | CH |
| 94 | CH₂CH₃ | H | CN | H | CF₃ | N |
| 95 | H | CH₃ | CN | H | CF₃ | N |
| 96 | CH₂CH₃ | H | CN | F | CF₃ | CH |
| 97 | CH₂CH₃ | H | CF₃ | H | OCF₃ | CH |
| 98 | CH₃ | H | CF₃ | H | 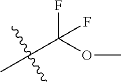 [SMILES: *C(F)(F)OC] | CH |
| 99 | CH₂CH₃ | H | CF₃ | H | 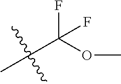 [SMILES: *C(F)(F)OC] | CH |
| 100 | CH₃ | H | CF₃ | H | 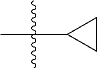 [SMILES: *C1CC1] | CH |
| 101 | H | OCH₃ | CHF₂ | H | CF₃ | CH |
| 102 | H | OCH₃ | F | H | CF₃ | CH |
| 103 | H | OCH₃ | CH₃ | H | CF₃ | CH |
| 104 | H | OCH₃ | OCHF₂ | H | CF₃ | CH |
| 105 | H | OCH₃ | OCF₃ | F | CF₃ | CH |
| 106 | H | OCH₃ | CF₃ | H | CF₃ | N |
| 107 | H | OCH₂CH₃ | CF₃ | H | CF₃ | N |
| 108 | H | OCH₂CH₃ | OCF₃ | H | CF₃ | CH |
| 109 | H | OCH₂CH₃ | CF₃ | H | CF₃ | CH |
| 110 | H | OCH₂CH₃ | CF₃ | F | CF₃ | CH |
| 111 | H | SCH₃ | CF₃ | H | CF₃ | N |
| 112 | H | 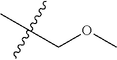 [SMILES: *COC] | CF₃ | F | CF₃ | H |
| 113 | H | 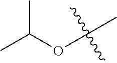 [SMILES: *OC(C)C] | CF3 | H | CF₃ | N |
| 114 | H | 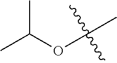 [SMILES: *OC(C)C] | CF₃ | H | CF₃ | CH |
| 115 | H | CH₃ | CHF₂ | H | CF₃ | CH |
| 116 | H | CH₃ | F | H | CF₃ | CH |
| 117 | H | CH₃ | OCHF₂ | F | CF₃ | CH |
| 118 | H | CH₃ | CH₃ | H | CF₃ | CH |
| 119 | H | CH₃ | CF₃ | H | CHF₂ | CH |
| 120 | H | CH₃ | CF₃ | F | CF₃ | CH |
| 121 | H | CH₃ | Cl | H | CF₃ | CH |
| 122 | H | CH₃ | OCF₃ | H | CF₃ | CH |
| 123 | H | CH₃ | OCF₃ | F | CF₃ | CH |
| 124 | H | CH₃ | OCHF₂ | H | CF₃ | CH |
| 125 | CH₃ | H | CHF₂ | H | CF₂CF₃ | CH |
| 126 | CH₃ | H | CF₃ | F | CF₂CF₃ | CH |
| 127 | CH₃ | H | OCHF₂ | H | CF₂CF₃ | CH |
| 128 | CH₂CH₃ | H | F | H | CHF₂ | CH |
| 129 | CH₂CH₃ | H | F | H | CF₃ | CH |
| 130 | CH₂CH₃ | H | Cl | H | CF₃ | CH |
| 131 | CH₂CH₃ | H | Cl | H | CHF₂ | CH |
| 132 | CH₂CH₃ | H | OCHF₂ | F | CF₃ | CH |
| 133 | CH₂CH₃ | H | OCHF₂ | H | CF₃ | CH |
| 134 | CH₂CH₃ | H | CH₃ | H | CF₃ | CH |
| 135 | CH₂CH₃ | H | CF₃ | F | CF₃ | CH |
| 136 | CH₂CH₃ | H | OCF₃ | F | CF₃ | CH |
| 137 | CH₂CH₃ | H | CHF₂ | H | CF₃ | CH |
| 138 | 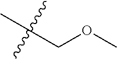 [SMILES: *COC] | H | F | H | CF₃ | CH |
| 139 | 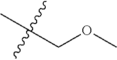 [SMILES: *COC] | H | Cl | H | CF₃ | CH |
| 140 | 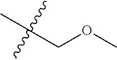 [SMILES: *COC] | H | CF₃ | H | 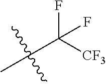 [SMILES: *C(F)(F)C(F)(F)F] | N |
| 141 | 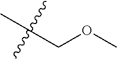 [SMILES: *COC] | H | CF₃ | H | CF₂CF₃ | CH |
| 142 | 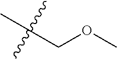 [SMILES: *COC] | H | CF₃ | H | CF₃ | N |
| 143 | 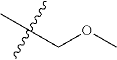 [SMILES: *COC] | H | CF₃ | F | CF₃ | CH |
| 144 | 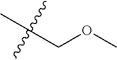 [SMILES: *COC] | H | CHF₂ | H | CF₃ | CH |
| 145 | 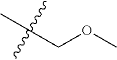 [SMILES: *COC] | H | OCF₃ | H | CF₃ | CH |
| 146 | 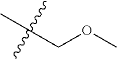 [SMILES: *COC] | H | OCF₃ | F | CF₃ | CH |
| 147 | 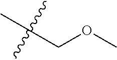 [SMILES: *COC] | H | OCHF₂ | F | CF₃ | CH |
| 148 | 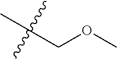 [SMILES: *COC] | H | OCHF₂ | H | CF₃ | CH |
| 149 | 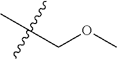 [SMILES: *COC] | H | CH₃ | H | CF₃ | CH |

5. A preparation method of the triazole compound according to claim 1, comprising:
subjecting the compound shown in the general formula II and the compound shown in the general formula III to substitution reaction to produce the compound shown in the general formula I, and the chemical reaction equation is as follows: 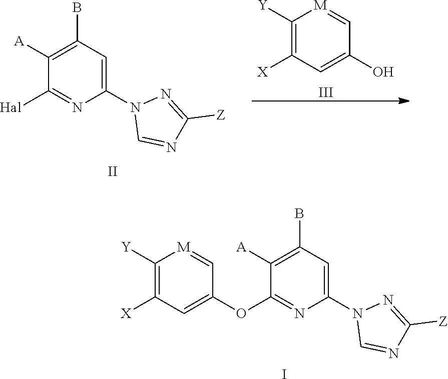
wherein, Hal represents halogen; the definitions of substituent groups A, B, M, X, Y, and Z are as shown in claim 1.

6. A An herbicidal composition, comprising (i) a herbicidally effective amount of at least one of the triazole compounds according to claim 1.

7. The herbicidal composition according to claim 13, wherein the additional herbicide is selected from one or more of the following compounds:
(1) VLCFA inhibitors: acetochlor, S-metolachlor, pretilachlor, butachlor, mefenacet, anilofos, pyroxasulfone, flufenacet, fentrazamide, napropamide;
(2) Microtubule Assembly inhibitors: pendimethalin, butralin, trifluralin;
(3) PSII inhibitors: metribuzin, prometryn, atrazine, terbuthylazine, isoproturon, chlorotoluron;
(4) PPO inhibitors: flumioxazin, 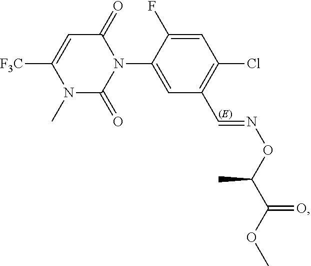 [SMILES: COC(=O)[C@@H](C)O/N=C/c1cc(-n2c(=O)cc(C(F)(F)F)n(C)c2=O)c(F)cc1Cl]
oxadiazon, oxadiargyl, sulfentrazone, pyraclonil, pentoxazone, saflufenacil, trifludimoxazin, 3-[2-chloro-5-[3,6-dihydro-3-methyl-2,6-dioxo-4-(trifluoromethyl)-1 (2H)-pyrimidinyl]-4-fluorophenyl]-4,5-dihydro-5-methyl-5-isoxazolecarboxylic acid ethyl ester, epyrifenacil, tiafenacil;
(5) HPPD inhibitors: isoxaflutole, fenpyrazone, 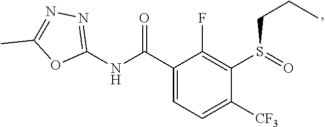 [SMILES: CCC[S@@](=O)c1c(C(F)(F)F)ccc(C(=O)Nc2nnc(C)o2)c1F]
tefuryltrione, benzobicyclon, bipyrazone;
(6) DOXP inhibitors: 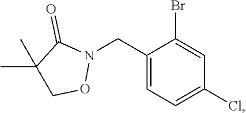 [SMILES: CC1(C)CON(Cc2ccc(Cl)cc2Br)C1=O]
bixlozone;
(7) Synthetic hormones: 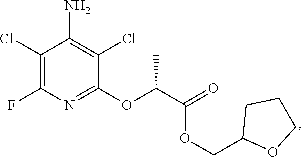 [SMILES: C[C@@H](Oc1nc(F)c(Cl)c(N)c1Cl)C(=O)OCC1CCCO1]
clopyralid, dicamba, halauxifen-methyl, 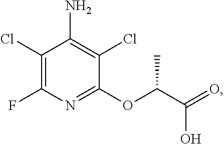 [SMILES: C[C@@H](Oc1nc(F)c(Cl)c(N)c1Cl)C(=O)O]
2,4-D-choline, 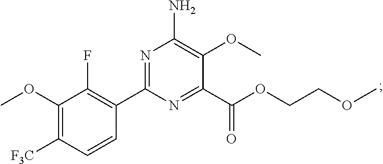
(8) ALS inhibitors: bensulfuron-methyl, pyrazosulfuron-ethyl, thifensulfuron-methyl, metazosulfuron, propyrisulfuron, thiencarbazone-methyl, triafamone, pyrimisulfan;
(9) FAT inhibitors: 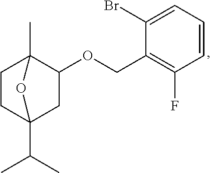 [SMILES: CC(C)C12CCC(C)(O1)C(OCc1c(F)cccc1Br)C2]
cinmethylin, oxaziclomefone;
(10) Lipid synthesis inhibitors: prosulfocarb;
(11) DHODH inhibitors: tetflupyrolimet;
(12) EPSPS inhibitors: glyphosate;
(13) GS inhibitors: glufosinate-ammonium;
(14) PSI inhibitors: paraquat dichloride, diquat dibromide monohydrate.

8. The herbicidal composition according to claim 13, wherein, the weight ratio of the effective ingredient in (i) and the additional herbicide in (iii) in the composition is 1:100~100:1, 1:80~80:1, 1:50~50:1, 1:30~30:1, 1:20~20:1, 1:10~10:1, 1:5~1:1, or 1:1~5:1.

9. A method for controlling weeds, comprising applying a herbicidally effective amount of at least one of the triazole compounds according to claim 1 to a plant or a weedy area.

10. A method controlling a weed among a useful crop, comprising applying at least one of the triazole compounds according to claim 1.

11. The preparation method according to claim 5, wherein the reaction is carried out in the presence of a base and a solvent.

12. The herbicidal composition according to claim 6, wherein the herbicidal composition also comprises (ii) a formulation auxiliary.

13. The herbicidal composition according to claim 6, wherein the herbicidal composition further comprises (iii) a herbicidally effective amount of one or more additional herbicides and/or safeners.

14. A method for controlling weeds, comprising applying a herbicidally effective amount of the herbicidal composition according to claim 6.

15. The method according to claim 10, wherein the useful crops are transgenic crops or crops treated by genome editing techniques.

16. A method controlling a weed among a useful crop, comprising applying the herbicidal composition according claim 6.

17. The method according to claim 16, wherein the useful crops are transgenic crops or crops treated by genome editing techniques.
